(19) United States
(12) Patent Application Publication
(10) Pub. No.: US 2016/0019337 A1
(43) Pub. Date: Jan. 21, 2016

(54) SUBTYPING LUNG CANCERS

(71) Applicant: HTG MOLECULAR DIAGNOSTICS, INC., Tucson, AZ (US)

(72) Inventors: Christopher Roberts, Tucson, AZ (US); Hui Wang, Tucson, AZ (US); Zhenquiang Lu, Tucson, AZ (US); Krishna Maddula, Tucson, AZ (US); Sam Rua, Tucson, AZ (US); Kevin Knapp, Tucson, AZ (US); Byron Lawson, Tucson, AZ (US); Debrah Thompson, Tucson, AZ (US); Michael Hrubiak, Tucson, AZ (US); Tyler Breedlove, Tucson, AZ (US); Vijay Modur, Actin, MA (US)

(73) Assignee: HTG Molecular Diagnostics, Inc., Tucson, AZ (US)

(21) Appl. No.: 14/772,038

(22) PCT Filed: Mar. 3, 2014
(86) PCT No.: PCT/US14/19967

§ 371 Date: Sep. 1, 2015

Related U.S. Application Data
(60) Provisional application No. 61/788,567, filed on Mar. 15, 2013.

(51) Int. Cl. G06F 19/20; C12Q 1/68

(52) U.S. Cl. CPC G06F 19/20; C12Q 1/6886; C12Q 2600/112; C12Q 2600/158; C12Q 2600/16

(57) ABSTRACT

This disclosure concerns the identification of biomarkers that are characteristic of squamous or non squamous (e.g., adenocarcinoma, large cell carcinoma, carcinoid tumor, sarcomatoid carcinoma) subtypes of non small cell lung cancer (NSCLC), clinically useful NSCLC classifiers, kits and arrays for distinguishing squamous and nonsquamous NSCLC subtypes, bioinformatic methods for determining clinically useful classifiers, and methods of use of each of the foregoing.

### CROSS REFERENCE TO RELATED APPLICATION

This application claims priority to U.S. Provisional Application No. 61/788,567 filed Mar. 15, 2013, herein incorporated by reference.

### FIELD

This disclosure concerns the identification of biomarkers and development of classifiers that are useful to differentiate among lung malignancies, including distinguishing the squamous subtype of non-small cell lung cancer (NSCLC) from non-squamous lung malignancies (e.g., adenocarcinoma, large cell carcinoma, carcinoid tumor, sarcomatoid carcinoma, and colon-tumor metastases).

### BACKGROUND

Lung cancer is the most common and deadly cancer in the world (Key et. al., Public Health Nutr., 7:187-200 (2004)) with approximately 1.2 million deaths every year (Parkin et al., CA Cancer J. Clin., 55:74-108 (2005)). Like many cancers, lung cancer is a heterogeneous disease (see FIG. 1A). Lung cancers are broadly classified into small cell lung cancers (SCLC) and non-small cell lung cancers (NSCLC) based upon the microscopic appearance of the tumor cells. The vast majority (80-85%) of lung cancers are NSCLC (Idowu et al., Pathol. Case Rev., 14:199-205 (2009)). A number of histological subtypes of NSCLC have been recognized, including, without limitation, adenocarcinomas, squamous cell carcinomas and large cell carcinomas.

Historically, physicians did not focus on subtyping NSCLCs because such determination did not provide clinically actionable information. With a significant shift in epidemiology as well as the availability of an increasing number of target-specific chemotherapeutic agents, subtype classification of NSCLC now is clinically relevant (Hirsch et al., J. Thorac. Oncol., 3:1468-1481 (2008)). For example, National Comprehensive Cancer Network (NCCN) Clinical Guidelines in Oncology for NSCLC (version 3.2012) indicate that (i) EGFR mutation and ALK testing are not routinely recommended for squamous NSCLC, (ii) Bevacizumab plus chemotherapy is not recommended for squamous NSCLC, (iii) Cisplatin/pemetrexed have superior efficacy and reduced toxicity for nonsquamous NSCLC, (iv) squamous first-line therapy is distinct from nonsquamous NSCLC therapy, and (v) Pemetrexed is not recommended for squamous NSCLC.

Current clinical and pathological practice for subtyping NSCLC consists of hematoxylin and eosin (H&E) staining followed by immunohistochemical (IHC) staining with antibodies specific for TTF-1 and p63 and, as deemed necessary, other proteins (e.g., chromogranin, synaptophysin). However, these practices have significant limitations, including improper diagnosis of other lung malignancies as NSCLC (see FIG. 1B; also, Idowa and Powers, Int. J. Clin. Exp. Pathol. 3(4): 367-385 (2010)) and markedly inconsistent results among physicians making the subtype diagnosis.

Diagnostic agreement can be estimated by calculating a k statistic, which is a measure of chance agreement. The k statistic ranges from complete disagreement (k=−1.0) to complete agreement (k=1.0), with a target minimum for clinical testing of k=0.7 (Landis and Koch, Biometrics, 33:159-174 (1977)). A recent study examining diagnoses of NSCLC into squamous or nonsquamous subtypes showed that expert pathologists were more likely to agree on the diagnosis than were community pathologists (k=0.64 for expert pathologists; k=0.41 for community pathologists; Arch. Pathol. Lab. Med., 137:32-40 (2013)). Among all pathologists in the study, clinical agreement decreased markedly as a function of decreasing diagnostic confidence (k decreasing from 0.78 to 0.28). Similarly, clinical agreement on the diagnosis of NSCLC subtype decreased for less differentiated tumors as compared to high/moderately differentiated tumors (k=0.6 for high/moderate differentiation; k=0.46 for poor differentiation). Notably, under almost all circumstances reported in the study, there was poor diagnostic agreement among pathologists on the subtyping of NSCLC as squamous or nonsquamous.

Based on both literature and current standard of care guidelines, accurate identification of NSCLC, including differentiation of squamous and nonsquamous NSCLC subtypes, is an established medical need at least for diagnostic, prognostic and therapeutic guidance. Reliable tools are needed to address this medical need (Check, “Pathologists picking up the pace in NSCLC,” CAP Today, June 2010).

### SUMMARY

Provided herein are methods and systems for characterizing a lung sample, such as a NSCLC sample, obtained from a subject. In some examples, one or more steps of the method are performed on a suitably programmed computer. In particular examples, the methods include obtaining, measuring or determining from the sample raw expression values for each of at least two biomarkers in any of Tables 2-4 and at least one normalization biomarker(s). The disclosure is not limited to particular methods of measuring expression values or levels. The raw expression values for each of the at least two biomarkers in Table 2 or 3 are normalized to the raw expression values for the at least one normalization biomarker(s), thereby generating or producing normalized expression values for each of the at least two biomarkers in any of Tables 2-4. The at least one normalization biomarker(s) can include a plurality of normalization biomarkers none of whose expression is statistically significantly different among a plurality of lung samples. Particular examples of normalization biomarkers are provided in Table 7.

The normalized expression values for each of the at least two biomarkers in any of Tables 2-4 are combined to generate an output value. For example, the combining can include weighting the expression level of the at least two biomarkers in any of Tables 2-4 with a constant predetermined for each of the at least two biomarkers in any of Tables 2-4, and summing the weighted expression levels of the at least two biomarkers in any of Tables 2-4 to generate the output value. The output value is compared to a cut-off value, such as a cut-off value determined by regression (e.g., logistic regression) analysis of normalized expression values for the at least two biomarkers in any of Tables 2-4 in a plurality of NSCLC samples known in advance to be squamous cell NSCLC or nonsquamous cell NSCLC.

The sample is then characterized. For example, the sample can be characterized as squamous cell NSCLC if the output value is on the same side of the cut-off value as the plurality of known squamous cell NSCLC samples or characterized as nonsquamous cell NSCLC if the output value is on the same side of the cut-off value as the plurality of known nonsquamous cell NSCLC samples. In some examples, the sample is characterized as nonsquamous cell NSCLC if the output value is below the cut-off value or as squamous cell NSCLC if the output value is above the cut-off value.

The method can include obtaining, measuring or determining from the sample additional raw expression values. For example, raw expression values for at least one colon metastasis biomarker in Table 5 can be determined and normalized to raw expression values for the at least one normalization biomarker(s) as described above. In some examples, the sample is identified as not NSCLC based on the normalized expression values for each of the at least one colon metastasis biomarker(s) in Table 5 and, optionally, the sample is removed from further NSCLC subtyping. In another or additional example, raw expression values for at least one pulmonary carcinoid/small cell lung cancer biomarker in Table 6 can be determined and normalized to raw expression values for the at least one normalization biomarker(s) as described above. In some examples, the sample is identified as not NSCLC based on the normalized expression values for each of the at least one pulmonary carcinoid/small cell lung cancer biomarker(s) in Table 6 and, optionally, the sample is removed from further NSCLC subtyping.

Also provided are methods of characterizing a lung sample obtained from a subject, for example to determine whether the sample originated from the colon (e.g., is a NSCLC or a colon metastasis). In some examples, the method includes obtaining, measuring or determining from the sample raw expression values for at least one colon metastasis biomarker in Table 5 (such as two or more of CDH17, LGALS4, CXCL17, SFTPA2, SCGB3A2, NAPSA, SFTPD, AQP4, SFTA3, SFTPC, CP, MUC13, HEPH, ZNF512B, and USH1C) and normalizing the raw expression values for each of the at least one colon metastasis biomarker(s) in Table 5 to the raw expression values for the at least one normalization biomarker(s) as described above. The sample can be identified as not NSCLC (e.g., is instead a colon metastasis) based on the normalized expression values for each of the at least one colon metastasis biomarker(s) in Table 5 and, optionally, the sample is removed from further NSCLC subtyping.

Also provided are methods of characterizing a lung sample obtained from a subject, for example to determine whether the sample is a NSCLC or is instead a pulmonary carcinoid or small cell lung cancer. In some examples, the method includes obtaining, measuring or determining from the sample raw expression values for at least one pulmonary carcinoid/small cell lung cancer biomarker in Table 6 (such as two or more of CHGA, TSPYL2, APLP1, CAMK2B, TAGLN3, and NCAM) and normalizing the raw expression values for each of the at least one pulmonary carcinoid/small cell lung cancer biomarker(s) in Table 6 to the raw expression values for the at least one normalization biomarker(s) as described above. The sample can be identified as not NSCLC (e.g., is instead a pulmonary carcinoid or small cell lung cancer) based on the normalized expression values for each of the at least one pulmonary carcinoid/small cell lung cancer biomarker(s) in Table 6 and, optionally, the sample is removed from further NSCLC subtyping.

The disclosure provides methods of determining gene expression in a lung sample. In particular examples, the method includes obtaining a lung sample from a subject and obtaining, measuring or determining in the sample expression levels of a plurality of genes comprising at least two of the biomarkers in any of Tables 2-4. A report is generated or produced that includes at least one of the gene expression levels in the sample, or a characterization of the sample as squamous NSCLC or nonsquamous NSCLC or neither. Such a method can further include determining in the sample expression levels of at least one normalization biomarker (such as one or more of those in Table 7).

Methods of subtyping NSCLC in a lung sample are disclosed herein. In some examples, such methods include obtaining, measuring or determining, in a lung sample obtained from a subject, an expression level of at least two biomarkers selected from KRT5, CAPN8, DSG3, IRF6, KCNK5, CSTA, CLCA2, TJP3, TP63, KRT7, MIR205HG, CLDN3, CGN, NKX2-1, SERPINB5, SLC2A1, KRT6B, KRT6A, TRIM29, S100A2, DeltaNP63, KRT13, MUC1, PKP1, RGL3, DSC3, PERP, and CALML3. Using the determined expression levels of the at least two biomarkers as an input, an output from an algorithm is calculated. Using the algorithm output, a determination is made as to whether the sample is squamous NSCLC, nonsquamous NSCLC or not NSCLC by comparing the output to a reference standard obtained from samples of known squamous and nonsquamous NSCLC subtypes. Such a method can further include normalizing the expression levels of the at least two biomarkers to the expression level of at least one normalization biomarker selected from the group consisting of at least one of EEF2, DDX17, HMGXB3, RPL19, RPS29 and/or RPSA; EEF2, DDX17, HMGXB3, RPL19, RPS29 and RPSA; or at least one gene expressed in the lung sample that is not the at least two biomarkers, and the expression of which does not significantly differ in a representative plurality of lung samples.

In some examples, the disclosed methods include providing to a user a report that includes the algorithm output or the determination that the sample is squamous NSCLC, nonsquamous NSCLC or not NSCLC.

In some examples, the disclosed methods include treating the subject based on the characterization of their lung sample. For example, if the lung sample is determined to be squamous NSCLC, the method can further include selecting the subject for chemotherapy treatment and/or treating the subject with chemotherapy. If the lung sample is determined to be non-squamous NSCLC, the method can further include selecting the subject for treatment with pemetrexed, bevacizumab, erlotinib, or crizotinib and/or treating the subject with pemetrexed, bevacizumab, erlotinib, or crizotinib.

Also provided are one or more non-transitory computer-readable media that include computer-executable instructions causing a computing system to perform the methods provided herein.

Systems for analyzing a sample (such as a sample obtained from a subject suspected of having NSCLC) obtained from a subject are also provided. Such systems can include a means (such as a NPP) for measuring raw expression values for each of at least two biomarkers in Table 2, 3, or 4 and at least one normalization biomarker(s), implemented rules for normalizing the raw expression values for each of the at least two biomarkers in Table 2, 3, or 4 to the raw expression values for the at least one normalization biomarker(s) to produce normalized expression values for each of the at least two biomarkers in Table 2, 3, or 4, implemented rules for combining the normalized expression values for each of the at least two biomarkers in Table 2, 3, or 4 to generate an output value, implemented rules for comparing the output value to a cut-off value (e.g., wherein the cut-off value was determined by regression or machine learning (e.g., support vector machine) analysis of normalized expression values for the at least two biomarkers in Table 2, 3, or 4 in a plurality of NSCLC samples known in advance to be squamous cell NSCLC or nonsquamous cell NSCLC), and/or means for implementing the rules (such as a computer or algorithm), wherein the sample is characterized as squamous cell NSCLC if the output value is on the same side of the cut-off value as the plurality of known squamous cell NSCLC samples or is characterized as nonsquamous cell NSCLC if the output value is on the same side of the cut-off value as the plurality of known nonsquamous cell NSCLC samples. In some examples, the normalized expression values for the plurality of NSCLC samples known in advance to be squamous cell NSCLC or nonsquamous cell NSCLC are stored values. In some examples, the normalized expression values for the plurality of NSCLC samples known in advance to be squamous cell NSCLC or nonsquamous cell NSCLC are measured from control samples by said means for measuring. It is to be understood that “raw” expression values as used throught this disclosure may have been, but need not be, routinely transformed data such as log-transformed data (e.g., log-2 transformed data).

In one example, such systems include a means (such as a NPP) for measuring raw expression value(s) for at least one colon metastasis biomarker in Table 5, implemented rules for normalizing the raw expression values for each of the at least one colon metastasis biomarker in Table 5 to the raw expression values for the at least one normalization biomarker(s) to produce normalized expression values for each of the at least one colon metastasis biomarker in Table 5, and means for implementing the rules (such as a computer or algorithm), wherein the sample is characterized as not NSCLC based on the normalized expression values for each of the at least one colon metastasis biomarker(s) in Table 5.

In one example, such systems include a means (such as an NPP) for measuring raw expression value(s) for at least one pulmonary carcinoid/small cell lung cancer biomarker in Table 6, implemented rules for normalizing the raw expression values for each of the at lest one pulmonary carcinoid/small cell lung cancer biomarker in Table 6 to the raw expression values for the at least one normalization biomarker(s) to produce normalized expression values for each of the at least one pulmonary carcinoid/small cell lung cancer biomarker in Table 6, and means for implementing the rules (such as a computer or algorithm), wherein the sample is characterized as not NSCLC based on the normalized expression values for each of the at least one pulmonary carcinoid/small cell lung cancer biomarker in Table 6. The disclosed systems can also incude a means for providing the output (such as a visual or audible output), such as whether the sample is characterized as squamous cell NSCLC or nonsquamous cell NSCLC, or whther the sample is characterized as NSCLC or not. Examples of such means include a computer, algorithm, monitor, tablet, printer and the like.

The disclosure provides arrays which can be used with the disclosed methods. For example, an array can include at least three addressable locations (such as at least 5, at least 10, at least 20, at least 30, at least 40, for example 3, 5, 15, 20, 25, 30, 40, 47, 50 or 100 addressable locations), wherein each location includes immobilized capture probes having the same specificity, and wherein each location includes capture probes having specificity different than capture probes at each other location. The capture probes at two of the at least three locations are capable of directly or indirectly specifically hybridizing a biomarker listed in any of Tables 2-4 (such as all of the biomarkers in Table 3), and the capture probes at one of the at least three locations is capable of directly or indirectly specifically hybridizing a normalization biomarker listed in Table 7 (such as the first six or all 11 of the biomarkers in Table 7), wherein the specificity of each capture probe is identifiable by the addressable location the array. In some examples, such an array further includes additional addressable locations, such as those that include capture probes capable of directly or indirectly specifically hybridizing to at least one colon metastasis biomarker listed in Table 5 (such as SFTPB, CLRN3, CDH17, LGALS4, and CXCL17), and/or capture probes capable of directly or indirectly specifically hybridizing to at least one pulmonary carcinoid/small cell lung cancer biomarker listed in Table 6 (such as CHGA, TSPYL2, APLP1, CAMK2B, TAGLN3, and NCAM1). In one example, the at least three addressable locations each are a separately identifiable bead or a channel in a flow cell. In a specific example, the array includes immobilized capture probes capable of directly or indirectly specifically hybridizing with all 28 biomarkers listed in Table 3 and the first 6 normalization biomarkers in Table 7. In some examples, the array also includes immobilized capture probes capable of directly or indirectly specifically hybridizing with a positive control and/or immobilized capture probes capable of directly or indirectly specifically hybridizing with a negative control. In a particular example, the capture probe(s) indirectly hybridize with the target (such as the at least two biomarkers listed in any of Tables 2-4 and the at least one normalization biomarker in Table 7) through a nucleic acid programming linker, wherein the programming linker is a hetro-bifunctional linker which has a first portion complementary to the capture probe(s) and a second portion complementary to a nuclease protection probe (NPP), wherein the NPP is complementary to a target (such as one of the at least two biomarkers listed in any of Tables 2-4 or the at least one normalization biomarker in Table 7).

Also provided are kits that include one or more of the arrays provided herein, which can further include one or more of a container containing lysis buffer; a container containing a nuclease specific for single-stranded nucleic acids; a container containing a plurality of nucleic acid programming linkers; a container containing a plurality of NPPs; a container containing a plurality of the bifunctional detection linkers; a container containing a detection probe that specifically binds the bifunctional detection linkers; and a container containing a detection reagent.

The foregoing and other features of the disclosure will become more apparent from the following detailed description of several embodiments, which proceeds with reference to the accompanying figures.

### BRIEF DESCRIPTION OF THE DRAWINGS

FIGS. 1A and 1B diagram (A) three major categories of lung malignancies and known cancer subtypes within each such category, and (B) cancer subtypes found among lung samples diagnosed or misdiagnosed (i.e., “Others”) as NSCLC and the relative percentage occurrence of each subtype.

FIG. 2 is a process diagram for a representative NSCLC squamous/nonsquamous classifier. Steps outlined in dotted lines are optional. If non-NSCLC samples (e.g., colon metastases and/or small cell lung cancer (SMC) and pulmonary carcinoids) optionally are identified, such samples may be identified in any order (e.g., colon metastates prior to SMC and pulmonary carcinoids or vice versa) or contemporaneously.

FIG. 3 are schematics for three ArrayPlates (Array 1, Array 2, and Array 3) used to develop DataSet 1 (see Example 1). Each array contained 96-wells and each well contained 47 spatially identifiable positions. The left most column of each array schematic shows the position of each gene as identified by its name and GenBank Accession No.

FIG. 4 shows a representative layout of positions in each well of a 96-well ArrayPlate.

FIG. 5 is a bar graph showing the number of the identified sample types (LC=large cell lung carcinoma; ADE=NSCLC adenocarcinoma; SQ=NSCLC squamous cell carcinoma) obtained from each of the five vendors.

FIG. 6 is a bar graph showing the number of times 26 of the genes in the representative 28-gene set (all but S100A2 and DeltaNp63-encoding variants of TP63) was identified as significantly differentially expressed between NSCLC squamous and nonsquamous subtypes in independent data sets. Almost half (12 of 26) of the genes were identified in all six independent data sets as significantly differentially expressed between the subject groups.

FIG. 7 show box and whisker plots for the indicated normalizer genes. The distributions of gene expression values (y-axis) in the identified sample types (x-axis) are shown. The bottom and top and line within the box show the upper and lower quartiles and median, respectively, and the whiskers show the minimum and maximum of all the data. Abbreviations: Colorect=colorectal (primarily colon adenocarcinoma); Lung Can=lung cancer; nSq=non-squamous lung carcinoma; Sq=squamous cell lung carcinoma; Ad=lung adenocarcinoma, LC=large cell lung carcinoma.

FIG. 8 shows a process map for obtaining consensus labeling for NSCLC samples.

FIG. 9 is a block diagram of an exemplary automation system for implementing selected disclosed method embodiments.

FIG. 10 is an exemplary sample preparation workflow for an automation system embodiment.

FIG. 11 is a workflow diagram for an automation system embodiment.

FIG. 12 is a schematic of a liquid-handling processor useful in the automation system embodiment.

FIGS. 13A and 13B are schematics of an exemplary pipetting manifold useful in a processor of the automation system embodiment.

FIG. 14 is a block diagram of an exemplary plate reader (or imager) useful in the automation system embodiment.

FIG. 15 is a schematic of the automation system software.

FIG. 16 is a schematic showing various treatment options presently known for NSCLC patients and the different regimes for such patients depending upon the cancer stage and whether their NSCLC is the squamous or nonsquamous subtype.

FIG. 17 is a plot showing the results of a representative support vector machine classifier used to subtype 27 NSCLC samples as squamous (e) or nonsquamous (*). Each sample is identified on the x-axis. The likelihood that a sample would be classified as nonsquamous (ADE) NSCLC from highest (1.0) to lowest (0.0) is shown on the y-axis. Samples above the line at 0.5 ADE Prediction Score were classified adenocarcinoma (nonsquamous) and samples below such line were classified squamous, which matched in all cases the adjudicated labeling for these samples.

FIG. 18 is a graph showing the results of squamous/nonsquamous NSCLC classifier prediction scores on mixed lung samples. The name of the respective mixed sample is shown on the x-axis (see Table 10 for sample details). The likelihood that a sample would be classified as nonsquamous (ADE) NSCLC from highest (1.0) to lowest (0.0) is shown on the y-axis. Samples above the line at 0.5 ADE Prediction Score were called adenocarcinoma (nonsquamous) and samples below such line were called squamous for purposes of Example 6.

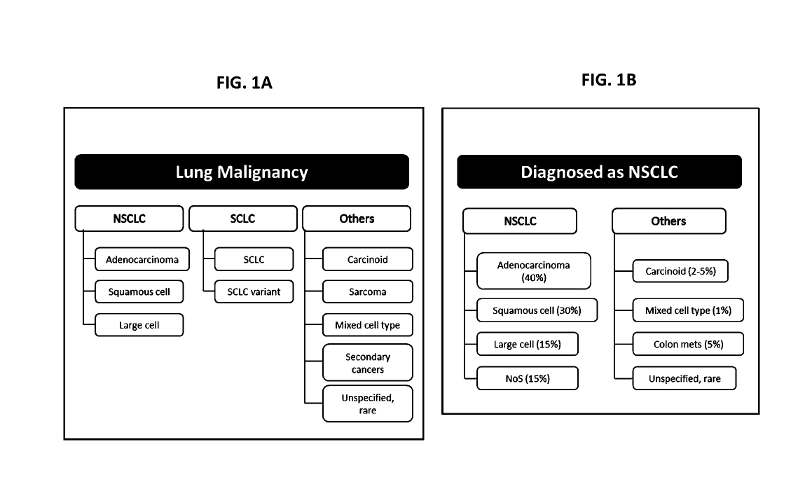

### SEQUENCES

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file in the form of the file named “Sequence.txt” (˜8 kb), which was created on Mar. 3, 2014, and which is incorporated by reference herein. In the accompanying sequence listing:

SEQ ID NOS: 1-47 provide NPP sequences that can be used to measure expression of the disclosed biomolecules.

### DETAILED DESCRIPTION

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The singular terms “a,” “an,” and “the” include plural referents unless context clearly indicates otherwise. Similarly, the word “or” is intended to include “and” unless the context clearly indicates otherwise. “Comprising” means “including.” Hence “comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety, as are the GenBank Accession numbers (for the sequence present on Mar. 15, 2013). In case of conflict, the present specification, including explanations of terms, will control. In addition, the materials, methods, and examples are illustrative only and not intended to be limiting.

Except as otherwise noted, the methods and techniques of the present disclosure are generally performed according to conventional methods well known in the art and as described in various general and more specific references that are cited and discussed throughout the present specification. See, e.g., Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999; each of which is specifically incorporated herein by reference in its entirety.

### Methods and Compositions for Characterizing Lung Malignancies

Thanks to advances in molecular biology and molecular medicine, it now is well accepted that “cancer” is a very heterogeneous collection of diseases generally characterized by dysregulated cell growth. Such heterogeneity creates many challenges for medical scientists and clinicians. Foremost among those challenges is the need to identify clinically relevant groups of cancer patients so that members of such groups can be treated in the most safe, efficient and effective manner(s).

The many cancer phenotypes result from corresponding patterns of gene expression in the affected cells, tissues and/or system. A (possibly, the most) powerful way to address the challenge of cancer heterogeneity is to match cancer phenotypes to clinically relevant gene expression patterns, as described below in more detail for distinguishing the squamous subtype of non-small cell lung cancer (NSCLC) from non-squamous lung malignancies (e.g., adenocarcinoma, large cell carcinoma, carcinoid tumor, sarcomatoid carcinoma, and colon-tumor metastases).

### Preparing to Obtain Gene Expression Data

Gene expression is the process by which information encoded in the genome (gene) is transformed (e.g., via transcription and translation processes) into corresponding gene products (e.g., RNA and protein), which function interrelatedly to give rise to a set of characteristics (aka, phenotype). For purposes of this disclosure, gene expression may be measured by any technique known now or in the future. Commonly, gene expression is measured by detecting the products of the genes (e.g., RNA and/or protein) expressed in samples collected from subjects of interest.

### Subjects and Samples

Appropriate samples for use in the methods disclosed herein include any biological sample from the lung and/or containing cells (e.g., NSCLC cells) from the lung or cells found in the lung (e.g., colon cancer metastases) for which information about gene or protein expression (such as those in any of Tables 2-8) is desired. Samples include those obtained from a subject, such as clinical samples obtained from a subject (including samples from a healthy or apparently healthy human subject or a human patient affected by a condition or disease to be diagnosed or investigated, such as lung cancer or, more particularly, NSCLC). In some embodiments, a biological sample previously has been diagnosed as NSCLC (or containing NSCLC) by histology or a clinical method (e.g., IHC or in situ hybridization (ISH)) other than described herein. In some examples, a prior-used method (such as histopathology or immunohistochemistry) was unable to reliably determine if the lung sample was squamous NSCLC or nonsquamous NSCLC.

Exemplary samples include, without limitation, cells, cell lysates, cytocentrifuge preparations, cytology smears, tissue biopsies (e.g., lung tissue biopsy, such as a core biopsy), fine-needle aspirates, and/or tissue sections (e.g., cryostat tissue sections and/or paraffin-embedded tissue sections). In one example a sample collected from the lung includes NSCLC cells or suspected NSCLC cells or, more particularly, previously has been diagnosed (e.g., using IHC or non-RNA-based method) as NSCLC. In particular examples, samples are used directly (e.g., fresh or frozen) or can be preserved prior to use, for example, by fixation (e.g., formalin fixation (such as, neutral buffered formalin, zinc formalin and acid formalin), ethanol fixation) and/or by embedding in a solid medium. Embedding media, typically, are inert, able to repel moisture and able to penetrate tissue (e.g., wax). Some useful samples are formalin-fixed, paraffin-embedded (FFPE) tissue samples. In specific examples, a lung tissue sample to be analyzed is fixed or, more particularly, fixed and wax- (paraffin-) embedded.

Standard techniques for acquisition of described samples are available. See, for example Tubbs and Stoler, Cell and Tissue Based Molecular Pathology, Philadelphia: Churchill Livingstone (2009), and Principles & Practice of Lung Cancer: The Official Reference Text of the IASLC, Fourth Edition, ed. by Pass, Carbone, Johnson, Minna, Scagliotti and Turrisi, Philadelphia: Lippincott Williams & Wilkins, a Wolters Kluwer business (2010). In some examples, a sample is a lung sample obtained, for example, by bronchoscopic biopsy, needle biopsy, open biopsy, video-assisted thoracoscopic surgery (VATS), thoracentesis, bronchiolar lavage (BAL), induced sputum, or brush cytology. It will appreciated that any method of obtaining a sample (such as tissue) from a subject can be utilized, and that the selection of the method used will depend upon various factors such as the type of tissue, age of the subject, or procedures available to the practitioner.

In some embodiments, a sample is a lysate of cells and/or tissue obtained from the lung. Cell lysate contains many of the proteins and nucleic acids contained in a cell, and include for example, the biomarkers shown in any of Tables 2-8. Methods for obtaining or preparing a cell lysate are well known in the art and can be found for example in Ausubel et al. (In Current Protocols in Molecular Biology, John Wiley & Sons, New York, 1998). In some examples, cells in the sample are lysed or permeabilized in an aqueous solution (for example using a lysis buffer). The aqueous solution or lysis buffer may include detergent (such as sodium dodecyl sulfate) and one or more chaotropic agents (such as formamide, guanidinium HCl, guanidinium isothiocyanate, or urea). The solution may also contain a buffer (for example SSC). In some examples, the lysis buffer includes about 8% to 60% formamide (v/v) about 0.01% to 0.5% SDS, and about 0.5-6×SSC (for example, about 3×SSC). The buffer may optionally include tRNA at about 0.001 to about 2.0 mg/ml or a ribonuclease. The lysis buffer may also include a pH indicator, such as Phenol Red. Cells are incubated in the aqueous solution for a sufficient period of time (such as about 1 minute to about 60 minutes, for example about 5 minutes to about 20 minutes, or about 10 minutes) and at a sufficient temperature (such as about 22° C. to about 115° C., for example, about 37° C. to about 105° C., or about 50° C. to about 95° C. or about 65° C. to about 100° C.) to lyse or permeabilize the cell. In some examples, lysis is performed at about 50° C., 65° C., or 95° C., for example if the nucleic acid to be detected is RNA. In other examples, lysis is performed at about 105° C., for example if the nucleic acid to be detected is DNA. In some examples, lysis conditions can be such that genomic DNA is not accessible to the probes whereas RNA (for example, mRNA) is, or such that the RNA is destroyed and only the DNA is accessible for probe hybridization. In some examples, the crude cell lysis is used directly without further purification.

### Control Samples

Control samples are contemplated by some disclosed methods, and include any suitable control sample against which to compare expression of a biomarker shown in any of Tables 2-8. In some embodiments, the control sample is non-tumor tissue, such as a plurality of non-tumor tissue samples. In one example, non-tumor tissue is tissue known to be benign, such as histologically normal lung tissue. In some examples, non-tumor tissue includes a lung sample that appears normal; that is, it has the absence of cellular dysplasia or other known disease (e.g., lung cancer, such as NSCLC) indicators. In some examples, the non-tumor tissue is obtained from the same subject, such as non-tumor tissue that is adjacent or even distant from a lung malignancy (such as NSCLC). In other examples, the non-tumor tissue is obtained from a healthy control subject or several healthy control subjects. For example, non-tumor tissue can be obtained from a plurality of healthy control subjects (e.g., those not having any cancers, including lung cancer (e.g., NSCLC), such as samples containing normal lung (or colon) cells or tissues from a plurality of such subjects. In some embodiments, one or more (e.g., a plurality of) control samples are used to obtain a reference (e.g., normal control) value or ranges of values for expression levels of the biomarkers shown in Tables 2-8. In some embodiments, a reference value obtained from control samples may be a population central tendency (such as a mean, median or average), or reference range of values such as ±0.5, 1.0, 1.5 or 2.0 standard deviation(s) around a population central tendency.

### Sample Analytical Options

Some method embodiments use fixed samples (e.g., FFPE tissue samples). Fixation techniques may vary from site-to-site, country-to-country, investigator-to-investigator, etc. (Dissecting the Molecular Anatomy of Tissue, ed. by Emmert-Buck, Gillespie and Chuaqui, New York: Springer-Verlag, 244 pages (2010)) and may affect the integrity of and/or accessibility to the gene product(s) to be detected. In some such methods (e.g., involving PCR), RNA recovery (e.g., using reversible cross linking agents, ethanol-based fixatives and/or RNA extraction or purification (in whole or in part)) may be advantageous; while, in other representative methods (e.g., involving qNPA) RNA recovery is optional or RNA recovery expressly is not needed. Similarly, tissue conditioning can be used to recover protein gene products from fixed tissue and, thereby, aid in the detection of such protein products.

The percentage of tumor (e.g., NSCLC) in biological samples may vary; thus, in some disclosed embodiments, at least 5%, at least 10%, at least 25%, at least 50%, at least 75%, at least 80% or at least 90% of the sample area (or sample volume) or total cells in the sample are tumor (e.g., NSCLC). In other examples, samples may be enriched for tumor cells, e.g., by macrodissecting areas or cells from a sample that are or appear to be predominantly tumor (e.g., NSCLC). Optionally, a pathologist or other appropriately trained professional may review the sample (e.g., H&E-stained tissue section) to determine if sufficient tumor is present in the sample for testing and/or mark the area (e.g., most dense tumor area) to be macrodissected. In specific examples, macrodissection of tumor (e.g., NSCLC) avoids as much as possible necrotic and/or hemorrhagic areas. Samples useful in some disclosed methods will have less than 25%, 15%, 10%, 5%, 2%, or 1% necrosis by sample volume or area or total cells.

Sample load influences the amount and/or concentration of gene product (e.g., one or more of the biomarkers in Tables 2-8) available for detection. In particular embodiments, at least 1 ng, 10 ng, 100 ng, 1 ug, 10 ug, 100 ug, 500 ug, 1 mg total RNA, at least 1 ng, 10 ng, 100 ng, 1 ug, 10 ug, 100 ug, 500 ug, 1 mg total DNA, or at least 0.01 ng, 0.1 ng, 1 ng, 10 ng, 100 ng, 1 ug, 10 ug, 100 ug, 500 ug, or 1 mg total protein is isolated from and/or present in a sample (such as a sample lysate). Some embodiments use tissue samples (e.g., FFPE lung tissues) that are at least 3, 5, 8, or 10 μm (e.g., about 3 to about 10 μm) thick and/or at least 0.15, 0.2, 0.5, 1, 1.5, 2, 5 or 10 cm² in area. The concentration of sample suspended in buffer in some method embodiments is at least 0.006 cm²/ul (e.g., 0.15 cm² FFPE lung tissue per 25 uL of buffer (e.g., lysis buffer)).

### Genes and Gene Sets

Among the innovations disclosed herein are genes (also referred to as biomarkers) and, preferably, sets of genes (also referred to as gene signatures) useful for distinguishing subtypes of lung malignancies in lung samples (e.g., samples that have been diagnosed by other means as NSCLC). In particular embodiments, genes and gene sets are disclosed for (i) identifying colon cells present in lung samples (e.g., colon tumor cells that have metastasized to the lung) (see Table 5); (ii) identifying the group of small cell lung cancer cells and carcinoids in lung samples (see Table 6); and (iii) subtyping squamous and nonsquamous NSCLCs (see Tables 2-4). Also disclosed are genes and gene sets useful as normalizers (e.g., sample-to-sample controls) (see Table 7) for normal and/or diseased lung samples, such as pluralities of lung tumor samples. Where, in some examples, such plurality of samples includes (or may include) NSCLCs (e.g., adenocarcinomas and/or squamous cell carcinomas), small cell carcinomas, lung metastases of colon tumors, and/or pulmonary carcinoids. In other examples, described in detail elsewhere in this disclosure, the expression of such genes and sets of genes are useful in classifiers of lung malignancies and algorithms, and/or to design analyte-specific reagents (e.g., nucleic acid probes or antibodies) for arrays or other disclosed compositions.

The disclosed genes and gene sets, optionally, are useful in combination (e.g., in series); for example, lung samples (e.g., samples believed to be NSCLC) may be prescreened for colon metastases and/or small cell carcinomas and pulmonary carcinoids using the applicable genes or gene sets and such samples removed from further consideration or identified as “indeterminate” or “not NSCLC” or the like; then, remaining samples subtyped as squamous or nonsquamous NSCLC using the genes or gene sets useful for distinguishing squamous and nonsquamous NSCLC.

In some embodiments of the disclosed methods, determining the level of expression in a biological sample (such as a lung biopsy, including NSCLC sample and/or FFPE sample) includes detecting two or more gene products (e.g., RNA or protein) shown in any of Tables 2-4 (and in some examples also one or more gene products (e.g., RNA or protein) shown in any of Tables 2-4), for example by determining the relative or actual amounts of such nucleic acids in the sample, as described in detail elsewhere.

Specific embodiments useful for subtyping squamous and nonsquamous NSCLC, include, without limitation:
a. one or more (e.g., at least or fixed at two, three, four, five, six, seven, eight, nine, 10, 15, 20, 25 or all) of CALML3, CLCA2, CLDN3, CSTA, DSC3, DSG3, KRT13, KRT5, KRT6B, PKP1, TP63, TRIM29, KRT6A, NKX2-1, CAPN8, SERPINB5, CGN, MUC1, PERP, IRF6, KCNK5, SLC2A1, TJP3, KRT7, MIR205HG, RGL3, DeltaNp63, and/or S100A2; or
b. any gene set comprising or consisting of any one or any two (to the extent not duplicative) of the following two-gene combinations: [CALML3,CLCA2], [CALML3,CLDN3], [CALML3,CSTA], [CALML3,DSC3], [CALML3,DSG3], [CALML3,KRT13], [CALML3,KRT5], [CALML3,KRT6B], [CALML3,PKP1], [CALML3,TP63], [CALML3,TRIM29], [CALML3,KRT6A], [CALML3,NKX2-1], [CALML3,CAPN8], [CALML3,SERPINB5], [CALML3,CGN], [CALML3,MUC1], [CALML3,PERP], [CALML3,IRF6], [CALML3,KCNK5], [CALML3,SLC2A1], [CALML3,TJP3], [CALML3,KRT7], [CALML3,MIR205HG], [CALML3,RGL3], [CALML3,DeltaNp63], [CALML3,S100A2], [CLCA2,CLDN3], [CLCA2,CSTA], [CLCA2,DSC3], [CLCA2,DSG3], [CLCA2,KRT13], [CLCA2,KRT5], [CLCA2,KRT6B], [CLCA2,PKP1], [CLCA2,TP63], [CLCA2,TRIM29], [CLCA2,KRT6A], [CLCA2,NKX2-1], [CLCA2,CAPN8], [CLCA2,SERPINB5], [CLCA2,CGN], [CLCA2,MUC1], [CLCA2,PERP], [CLCA2,IRF6], [CLCA2,KCNK5], [CLCA2,SLC2A1], [CLCA2,TJP3], [CLCA2,KRT7], [CLCA2,MIR205HG], [CLCA2,RGL3], [CLCA2,DeltaNp63], [CLCA2,S100A2], [CLDN3,CSTA], [CLDN3,DSC3], [CLDN3,DSG3], [CLDN3,KRT13], [CLDN3,KRT5], [CLDN3,KRT6B], [CLDN3,PKP1], [CLDN3,TP63], [CLDN3,TRIM29], [CLDN3,KRT6A], [CLDN3,NKX2-1], [CLDN3,CAPN8], [CLDN3,SERPINB5], [CLDN3,CGN], [CLDN3,MUC1], [CLDN3,PERP], [CLDN3,IRF6], [CLDN3,KCNK5], [CLDN3,SLC2A1], [CLDN3,TJP3], [CLDN3,KRT7], [CLDN3,MIR205HG], [CLDN3,RGL3], [CLDN3,DeltaNp63], [CLDN3,S100A2], [CSTA,DSC3], [CSTA,DSG3], [CSTA,KRT13], [CSTA,KRT5], [CSTA,KRT6B], [CSTA,PKP1], [CSTA,TP63], [CSTA,TRIM29], [CSTA,KRT6A], [CSTA,NKX2-1], [CSTA,CAPN8], [CSTA,SERPINB5], [CSTA,CGN], [CSTA,MUC1], [CSTA,PERP], [CSTA,IRF6], [CSTA,KCNK5], [CSTA,SLC2A1], [CSTA,TJP3], [CSTA,KRT7], [CSTA,MIR205HG], [CSTA,RGL3], [CSTA,DeltaNp63], [CSTA,S100A2], [DSC3,DSG3], [DSC3,KRT13], [DSC3,KRT5], [DSC3,KRT6B], [DSC3,PKP1], [DSC3,TP63], [DSC3,TRIM29], [DSC3,KRT6A], [DSC3,NKX2-1], [DSC3,CAPN8], [DSC3,SERPINB5], [DSC3,CGN], [DSC3,MUC1], [DSC3,PERP], [DSC3,IRF6], [DSC3,KCNK5], [DSC3,SLC2A1], [DSC3,TJP3], [DSC3,KRT7], [DSC3,MIR205HG], [DSC3,RGL3], [DSC3,DeltaNp63], [DSC3,S100A2], [DSG3,KRT13], [DSG3,KRT5], [DSG3,KRT6B], [DSG3,PKP1], [DSG3,TP63], [DSG3,TRIM29], [DSG3,KRT6A], [DSG3,NKX2-1], [DSG3,CAPN8], [DSG3,SERPINB5], [DSG3,CGN], [DSG3,MUC1], [DSG3,PERP], [DSG3,IRF6], [DSG3,KCNK5], [DSG3,SLC2A1], [DSG3,TJP3], [DSG3,KRT7], [DSG3,MIR205HG], [DSG3,RGL3], [DSG3,DeltaNp63], [DSG3,S100A2], [KRT13,KRT5], [KRT13,KRT6B], [KRT13,PKP1], [KRT13,TP63], [KRT13,TRIM29], [KRT13,KRT6A], [KRT13,NKX2-1], [KRT13,CAPN8], [KRT13,SERPINB5], [KRT13,CGN], [KRT13,MUC1], [KRT13,PERP], [KRT13,IRF6], [KRT13,KCNK5], [KRT13,SLC2A1], [KRT13,TJP3], [KRT13,KRT7], [KRT13,MIR205HG], [KRT13,RGL3], [KRT13,DeltaNp63], [KRT13,S100A2], [KRT5,KRT6B], [KRT5,PKP1], [KRT5,TP63], [KRT5,TRIM29], [KRT5,KRT6A], [KRT5,NKX2-1], [KRT5,CAPN8], [KRT5,SERPINB5], [KRT5,CGN], [KRT5,MUC1], [KRT5,PERP], [KRT5,IRF6], [KRT5,KCNK5], [KRT5,SLC2A1], [KRT5,TJP3], [KRT5,KRT7], [KRT5,MIR205HG], [KRT5,RGL3], [KRT5,DeltaNp63], [KRT5,S100A2], [KRT6B,PKP1], [KRT6B,TP63], [KRT6B,TRIM29], [KRT6B,KRT6A], [KRT6B,NKX2-1], [KRT6B,CAPN8], [KRT6B,SERPINB5], [KRT6B,CGN], [KRT6B,MUC1], [KRT6B,PERP], [KRT6B,IRF6], [KRT6B,KCNK5], [KRT6B,SLC2A1], [KRT6B,TJP3], [KRT6B,KRT7], [KRT6B,MIR205HG], [KRT6B,RGL3], [KRT6B,DeltaNp63], [KRT6B,S100A2], [PKP1,TP63], [PKPETRIM29], [PKPEKRT6A], [PKPENKX2-1], [PKPECAPN8], [PKPESERPINB5], [PKP1,CGN], [PKPLMUC1], [PKP1,PERP], [PKPEIRF6], [PKPEKCNK5], [PKPESLC2A1], [PKPLTJP3], [PKPLKRT7], [PKPEMIR205HG], [PKPLRGL3], [PKP1,DeltaNp63], [PKP1,S100A2], [TP63,TRIM29], [TP63,KRT6A], [TP63,NKX2-1], [TP63,CAPN8], [TP63,SERPINB5], [TP63,CGN], [TP63,MUC1], [TP63,PERP], [TP63,IRF6], [TP63,KCNK5], [TP63,SLC2A1], [TP63,TJP3], [TP63,KRT7], [TP63,MIR205HG], [TP63,RGL3], [TP63,DeltaNp63], [TP63,S100A2], [TRIM29,KRT6A], [TRIM29,NKX2-1], [TRIM29,CAPN8], [TRIM29,SERPINB5], [TRIM29,CGN], [TRIM29,MUC1], [TRIM29,PERP], [TRIM29,IRF6], [TRIM29,KCNK5], [TRIM29,SLC2A1], [TRIM29,TJP3], [TRIM29,KRT7], [TRIM29,MIR205HG], [TRIM29,RGL3], [TRIM29,DeltaNp63], [TRIM29,S100A2], [KRT6A,NKX2-1], [KRT6A,CAPN8], [KRT6A,SERPINB5], [KRT6A,CGN], [KRT6A,MUC1], [KRT6A,PERP], [KRT6A,IRF6], [KRT6A,KCNK5], [KRT6A,SLC2A1], [KRT6A,TJP3], [KRT6A,KRT7], [KRT6A,MIR205HG], [KRT6A,RGL3], [KRT6A,DeltaNp63], [KRT6A,S100A2], [NKX2-1,CAPN8], [NKX2-1,SERPINB5], [NKX2-1,CGN], [NKX2-1,MUC1], [NKX2-1,PERP], [NKX2-1,IRF6], [NKX2-1,KCNK5], [NKX2-1,SLC2A1], [NKX2-1,TJP3], [NKX2-1,KRT7], [NKX2-1,MIR205HG], [NKX2-1,RGL3], [NKX2-1,DeltaNp63], [NKX2-1,S100A2], [CAPN8,SERPINB5], [CAPN8,CGN], [CAPN8,MUC1], [CAPN8,PERP], [CAPN8,IRF6], [CAPN8,KCNK5], [CAPN8,SLC2A1], [CAPN8,TJP3], [CAPN8,KRT7], [CAPN8,MIR205HG], [CAPN8,RGL3], [CAPN8,DeltaNp63], [CAPN8,S100A2], [SERPINB5,CGN], [SERPINB5,MUC1], [SERPINB5,PERP], [SERPINB5,IRF6], [SERPINB5,KCNK5], [SERPINB5,SLC2A1], [SERPINB5,TJP3], [SERPINB5,KRT7], [SERPINB5,MIR205HG], [SERPINB5,RGL3], [SERPINB5,DeltaNp63], [SERPINB5,S100A2], [CGN,MUC1], [CGN,PERP], [CGN,IRF6], [CGN,KCNK5], [CGN,SLC2A1], [CGN,TJP3], [CGN,KRT7], [CGN,MIR205HG], [CGN,RGL3], [CGN,DeltaNp63], [CGN,S100A2], [MUC1,PERP], [MUCEIRF6], [MUC1,KCNK5], [MUCLSLC2A1], [MUC1,TJP3], [MUC1,KRT7], [MUCEMIR205HG], [MUCERGL3], [MUC1,DeltaNp63], [MUC1,S100A2], [PERP,IRF6], [PERP,KCNK5], [PERP,SLC2A1], [PERP,TJP3], [PERP, KRT7], [PERP,MIR205HG], [PERP,RGL3], [PERP,DeltaNp63], [PERP,S100A2], [IRF6,KCNK5], [IRF6,SLC2A1], [IRF6,TJP3], [IRF6,KRT7], [IRF6,MIR205HG], [IRF6,RGL3], [IRF6,DeltaNp63], [IRF6,S100A2], [KCNK5,SLC2A1], [KCNK5,TJP3], [KCNK5,KRT7], [KCNK5,MIR205HG], [KCNK5,RGL3], [KCNK5,DeltaNp63], [KCNK5,S100A2], [SLC2A1,TJP3], [SLC2ALKRT7], [SLC2ALMIR205HG], [SLC2ALRGL3], [SLC2A1,DeltaNp63], [SLC2A1,S100A2], [TJP3,KRT7], [TJP3,MIR205HG], [TJP3,RGL3], [TJP3,DeltaNp63], [TJP3,S100A2], [KRT7,MIR205HG], [KRT7,RGL3], [KRT7,DeltaNp63], [KRT7,S100A2], [MIR205HG,RGL3], [MIR205HG,DeltaNp63], [MIR205HG,S100A2], [RGL3,DeltaNp63], [RGL3,S100A2], [DeltaNp63,S100A2]; or
c. Any gene set comprising or consisting of three-gene combinations that include any one of the two-gene sets in (b) with the addition of any one of the following genes into a set of three, non-duplicative genes: CALML3, CLCA2, CLDN3, CSTA, DSC3, DSG3, KRT13, KRT5, KRT6B, PKP1, TP63, TRIM29, KRT6A, NKX2-1, CAPN8, SERPINB5, CGN, MUC1, PERP, IRF6, KCNK5, SLC2A1, TJP3, KRT7, MIR205HG, RGL3, DeltaNp63, S100A2, DST, KRT17, NTRK2, PI13 (aka SERPINB13), SLC6A8, SPRR1A, SPRR1B, or SPRR3; or
d. one or more (e.g., at least or fixed at two, three, four, five, six, seven, eight, or nine) of KRT5, KRT6A, KRT6B, KRT13, KRT7, MUC1, TP63, NKX2-1, or DeltaNp63 or P40.

Specific embodiments useful for identifying (or classifying) colon-originating cells in the lung (e.g, colon tumor metastases), include:
e. one or more (e.g., at least or fixed at two, three, four, five, six, seven, eight, nine, 10, 15, or all) of SFTPB, CLRN3, CDH17, LGALS4, CXCL17, SFTPA2, SCGB3A2, NAPSA, SFTPD, AQP4, SFTA3, SFTPC, CP, MUC13, HEPH, ZNF512B, and/or USH1C; or
f. SFTPB, CLRN3, CDH17, LGALS4, and CXCL17; or
g. any gene set comprising or consisting of any gene set comprising or consisting of any one or any two (to the extent not duplicative) of the following two-gene combinations: [SFTPB,CLRN3], [SFTPB,CDH17], [SFTPB,LGALS4], [SFTPB,CXCL17], [SFTPB,SFTPA2], [SFTPB,SCGB3A2], [SFTPB,NAPSA], [SFTPB,SFTPD], [SFTPB,AQP4], [SFTPB,SFTA3], [SFTPB,SFTPC], [SFTPB,CP], [SFTPB,MUC13], [SFTPB,HEPH], [SFTPB,ZNF512B], [SFTPB,USH1C], [CLRN3,CDH17], [CLRN3,LGALS4], [CLRN3,CXCL17], [CLRN3,SFTPA2], [CLRN3,SCGB3A2], [CLRN3,NAPSA], [CLRN3,SFTPD], [CLRN3,AQP4], [CLRN3,SFTA3], [CLRN3,SFTPC], [CLRN3,CP], [CLRN3,MUC13], [CLRN3,HEPH], [CLRN3,ZNF512B], [CLRN3,USH1C], [CDH17,LGALS4], [CDH17,CXCL17], [CDH17,SFTPA2], [CDH17,SCGB3A2], [CDH17,NAPSA], [CDH17,SFTPD], [CDH17,AQP4], [CDH17,SFTA3], [CDH17,SFTPC], [CDH17,CP], [CDH17,MUC13], [CDH17,HEPH], [CDH17,ZNF512B], [CDH17,USH1C], [LGALS4,CXCL17], [LGALS4,SFTPA2], [LGALS4,SCGB3A2], [LGALS4,NAPSA], [LGALS4,SFTPD], [LGALS4,AQP4], [LGALS4,SFTA3], [LGALS4,SFTPC], [LGALS4,CP], [LGALS4,MUC13], [LGALS4,HEPH], [LGALS4,ZNF512B], [LGALS4,USH1C], [CXCL17,SFTPA2], [CXCL17,SCGB3A2], [CXCL17,NAPSA], [CXCL17,SFTPD], [CXCL17,AQP4], [CXCL17,SFTA3], [CXCL17,SFTPC], [CXCL17,CP], [CXCL17,MUC13], [CXCL17,HEPH], [CXCL17,ZNF512B], [CXCL17,USH1C], [SFTPA2,SCGB3A2], [SFTPA2,NAPSA], [SFTPA2,SFTPD], [SFTPA2,AQP4], [SFTPA2,SFTA3], [SFTPA2,SFTPC], [SFTPA2,CP], [SFTPA2,MUC13], [SFTPA2,HEPH], [SFTPA2,ZNF512B], [SFTPA2,USH1C], [SCGB3A2,NAPSA], [SCGB3A2,SFTPD], [SCGB3A2,AQP4], [SCGB3A2,SFTA3], [SCGB3A2,SFTPC], [SCGB3A2,CP], [SCGB3A2,MUC13], [SCGB3A2,HEPH], [SCGB3A2,ZNF512B], [SCGB3A2,USH1C], [NAPSA,SFTPD], [NAPSA,AQP4], [NAPSA,SFTA3], [NAPSA,SFTPC], [NAPSA,CP], [NAPSA,MUC13], [NAPSA,HEPH], [NAPSA,ZNF512B], [NAPSA,USH1C], [SFTPD,AQP4], [SFTPD,SFTA3], [SFTPD,SFTPC], [SFTPD,CP], [SFTPD,MUC13], [SFTPD,HEPH], [SFTPD,ZNF512B], [SFTPD,USH1C], [AQP4,SFTA3], [AQP4,SFTPC], [AQP4,CP], [AQP4,MUC13], [AQP4,HEPH], [AQP4,ZNF512B], [AQP4,USH1C], [SFTA3,SFTPC], [SFTA3,CP], [SFTA3,MUC13], [SFTA3,HEPH], [SFTA3,ZNF512B], [SFTA3,USH1C], [SFTPC,CP], [SFTPC,MUC13], [SFTPC,HEPH], [SFTPC,ZNF512B], [SFTPC,USH1C], [CP,MUC13], [CP,HEPH], [CP,ZNF512B], [CP,USH1C], [MUC13,HEPH], [MUC13,ZNF512B], [MUC13,USH1C], [HEPH,ZNF512B], [HEPH,USH1C], [ZNF512B,USH1C]; or
h. Any gene set comprising or consisting of three-gene combinations that include any one of the two-gene sets in (g) with the addition of any one of the genes in (e) into a set of three, non-duplicative genes.

Specific embodiments useful for identifying (or classifying) small cell carcinoma and pulmonary carcinoids in lung samples, include:
i. one or more (e.g., at least or fixed at two, three, four, five, or all six) of CHGA, TSPYL2, APLP1, CAMK2B, TAGLN3, or NCAM1;
j. any gene set comprising or consisting of any one or any two (to the extent not duplicative) of the following two-gene combinations: [CHGA,TSPYL2], [CHGA,APLP1], [CHGA,CAMK2B], [CHGA,TAGLN3], [CHGA,NCAM1], [TSPYL2,APLP1], [TSPYL2,CAMK2B], [TSPYL2,TAGLN3], [TSPYL2,NCAM1], [APLP1,CAMK2B], [APLPLTAGLN3], [APLP1,NCAM1], [CAMK2B,TAGLN3], [CAMK2B,NCAM1], or [TAGLN3,NCAM1]; or
k. any gene set comprising or consisting of any one or any two (to the extent not duplicative) of the following three-gene combinations: [CHGA,TSPYL2,APLP1], [CHGA,TSPYL2,CAMK2B], [CHGA,TSPYL2,TAGLN3], [CHGA,TSPYL2,NCAM1], [CHGA,APLPLCAMK2B], [CHGA,APLPLTAGLN3], [CHGA,APLP1,NCAM1], [CHGA,CAMK2B,TAGLN3], [CHGA,CAMK2B,NCAM1], [CHGA,TAGLN3,NCAM1], [TSPYL2,APLP1,CAMK2B], [TSPYL2,APLP1,TAGLN3], [TSPYL2,APLP1,NCAM1], [TSPYL2,CAMK2B,TAGLN3], [TSPYL2,CAMK2B,NCAM1], [TSPYL2,TAGLN3,NCAM1], [APLP1,CAMK2B,TAGLN3], [APLP1,CAMK2B,NCAM1], [APLP1,TAGLN3,NCAM1], or [CAMK2B,TAGLN3,NCAM1].

As discussed elsewhere in detail, the expression of normalizing genes (also referred to as housekeeper genes or endogenous controls or the like) is one (but not the only) useful way to correct for non-biological or sample-to-sample variation that arises in multiplexed gene expression assays. The expression of normalizing genes is not significantly different among the samples used in the particular assay. Specific housekeeper genes disclosed herein, include:
l. one or more (e.g., at least or fixed at two, three, four, five, or all six) of EEF2, DDX17, HMGXB3, RPL19, RPSA or RPS29;
m. one or more (e.g., at least or fixed at two, three, four, or all five) of RPL37A, RPL41, CFL1, MTND4, or OAZ1; or
n. any gene set comprising or consisting of any one or any two (to the extent not duplicative) of the following two-gene combinations: [EEF2,DDX17], [EEF2,HMGXB3], [EEF2,RPL19], [EEF2,RPSA], [EEF2,RPS29], [DDX17,HMGXB3], [DDX17,RPL19], [DDX17,RPSA], [DDX17,RPS29], [HMGXB3,RPL19], [HMGXB3,RPSA], [HMGXB3,RPS29], [RPL19,RPSA], [RPL19,RPS29], or [RPSA,RPS29]; or
o. any gene set comprising or consisting of any one or any two (to the extent not duplicative) of the following three-gene combinations: [EEF2,DDX17,HMGXB3], [EEF2,DDX17,RPL19], [EEF2,DDX17,RPSA], [EEF2,DDX17,RPS29], [EEF2,HMGXB3,RPL19], [EEF2,HMGXB3,RPSA], [EEF2,HMGXB3,RPS29], [EEF2,RPL19,RPSA], [EEF2,RPL19,RPS29], [EEF2,RPSA,RPS29], [DDX17,HMGXB3,RPL19], [DDX17,HMGXB3,RPSA], [DDX17,HMGXB3,RPS29], [DDX17,RPL19,RPSA], [DDX17,RPL19,RPS29], [DDX17,RPSA,RPS29], [HMGXB3,RPL19,RPSA], [HMGXB3,RPL19,RPS29], [HMGXB3,RPSA,RPS29], [RPL19,RPSA,RPS29]; or
p. any non-duplicative gene set comprising or consisting of (i) three gene combinations between (n) and (l) or (m), or (ii) four gene combinations between (o) and (l) or (m).

### Obtaining Gene Expression Information

A variety of techniques are (or may become) available for measuring gene expression in a sample of interest. However, the disclosure is not limited to particular methods of obtaining, measuring, detecting gene expression. Many such techniques involve detecting the products of the genes (e.g., nucleic acids (such as RNA) and/or protein) expressed in such samples. It may also be (or become) possible to directly detect the activity of a gene or of chromosomal DNA (e.g., transcription rate) independent of measuring its resultant gene products and such techniques also are useful in methods disclosed herein.

### Detecting Nucleic-Acid Gene Products

Nucleic-acid gene products are, as the name suggests, products of gene expression that are nucleic acids. Exemplary nucleic acids include DNA or RNA, such as cDNA, protein-coding RNA (e.g., mRNA) or non-coding RNA (e.g., long, non-coding (Mc) RNA). Base pairing between complementary strands of RNA or DNA (i.e., nucleic acid hybridization) forms all or part of the basis for a large representative class of techniques for detecting nucleic-acid gene products. Other representative detection techniques involve nucleic acid sequencing, which may or may not involve hybridization steps and/or bioinformatics steps (e.g., to associate nucleic acid sequence information to its corresponding gene). These and other methods of detecting nucleic acids are known in the art and, while representative techniques are described herein, this disclosure is not intended to be limited to particular methods of nucleic acid detection.

Optional Nucleic Acid Isolation

In some examples, nucleic acids are isolated or extracted from the lung sample prior to contacting such nucleic acids in the sample with a complementary nucleic acid probe and/or otherwise detecting such nucleic acids in the sample. Nucleic acids (such as RNA (e.g., mRNA or lncRNA) or DNA) can be isolated from the sample according to any of a number of methods. Representative methods of isolation and purification of nucleic acids are described in detail in Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993) and Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993). Representative methods for RNA (e.g., mRNA or lncRNA) extraction similarly are well known in the art and are disclosed in standard textbooks of molecular biology, including Ausubel et al., Current Protocols of Molecular Biology, John Wiley and Sons (1997).

Specific methods can include isolating total nucleic acid from a sample using, for example, an acid guanidinium-phenol-chloroform extraction method and/or isolating polyA+ mRNA by oligo dT column chromatography or by (dT)n magnetic beads (see, for example, Sambrook et al, Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, N.Y. (1987)). In other examples, nucleic acid isolation can be performed using purification kit, buffer set and protease from commercial manufacturers, such as QIAGEN® (Valencia, Calif.), according to the manufacturer's instructions. For example, total RNA from cells (such as those obtained from a subject) can be isolated using QIAGEN® RNeasy mini-columns Other commercially available nucleic acid isolation kits include MASTERPURE® Complete DNA and RNA Purification Kit (EPICENTRE® Madison, Wis.), and Paraffin Block RNA Isolation Kit (Ambion, Inc.). Total RNA from tissue samples can be isolated using RNA Stat-60 (Tel-Test). RNA prepared from tumor or other biological sample can be isolated, for example, by cesium chloride density gradient centrifugation. Methods for RNA extraction from paraffin embedded tissues are disclosed, for example, in Rupp and Locker, Biotechniques 6:56-60 (1988), and De Andres et al., Biotechniques 18:42-44 (1995).

After isolation or extraction of nucleic acids (e.g., RNA (such as mRNA or lncRNA) or DNA) from a sample, any of a number of optional other steps may be performed to prepare such nucleic acids for detection, including measuring the concentration of the isolated nucleic acid, repair (or recovery) of degraded or damaged RNA, RNA reverse transcription, and/or amplification of RNA or DNA.

In other examples, a sample (e.g., FFPE lung tissue sample) is suspended in a buffer (e.g., lysis buffer) and nucleic acids (such as RNA or DNA) present in the suspended sample are not isolated or extracted (e.g., purified in whole or in part) from such suspended sample and are contacted in such suspension with one or more complementary nucleic acid probe(s) (e.g., nuclease protection probes); thereby, eliminating a need for isolation or extraction of nucleic acids (e.g., RNA) from the sample. This embodiment is particularly advantageous where the nucleic acids (such as RNA or DNA) present in the suspended sample are crosslinked or fixed to cellular structures and are not readily isolatable or extractable. Relatively short (e.g., less than 100 base pairs, such as 75-25 base pairs or 50-25 base pairs) probes for which no extension of such probe is required for detection are useful in some non-extraction method embodiments. An ordinarily skilled artisan will appreciate that methods requiring probe extension (e.g., PCR or primer extension) are not reliable where the nucleic acid template (e.g., RNA) for such extension is degraded or otherwise inaccessible. Specific methods (e.g., qNPA) for detecting nucleic acids (e.g., RNA) in a sample without prior extraction of such nucleic acids are described in detail elsewhere in this disclosure.

Nucleic Acid Hybridization

In some examples, determining the expression level of a disclosed biomarker (such as those in Tables 2-6) or normalization biomarker (Table 7) in the methods provided herein can include contacting the sample with a plurality of nucleic acid probes (such as a nuclease protection probe, NPP, or adjoining ligatable probes) or paired amplification primers, wherein each probe (or set of ligatable probes) or paired primers in the plurality is/are specific and complementary to one of at least two biomarkers in Tables 2-6 or a or normalization biomarker in Table 7, under conditions that permit the plurality of nucleic acid probes or paired primers to hybridize to its/their complementary biomarker in Tables 2-7. In one example, the method can also include after contacting the sample with the plurality of nucleic acid probes (such as NPPs), contacting the sample with a nuclease that digests single-stranded nucleic acid molecules. In other examples, each of the at least two biomarkers in Tables 2-6, or a or normalization biomarker in Table 7, is contacted with a “probe set” that consists of multiple (e.g., 2, 3, 4, 5, or 6) probes specific for each such biomarker, which design can be useful, for example, to increase the signal obtained from such gene product or to detect multiple variants of the same gene product.

In some examples, variable (Tables 2-6) or normalization (Table 7) nucleic acids are detected by nucleic acid hybridization. Nucleic acid hybridization involves providing a denatured probe and target nucleic acid (e.g., those in Tables 2-7) under conditions where the probe and its complementary target can form stable hybrid duplexes through complementary base pairing. In some examples, the nucleic acids that do not form hybrid duplexes are then removed (e.g., washed away, digested by nuclease or physically removed) leaving the hybridized nucleic acids to be detected, typically through detection of a (directly or indirectly) attached detectable label. In specific examples, nucleic acids that do not form hybrid duplexes, such as any excess probe that does not hybridize to its respective target, and the regions of the target sequence that are not complementary to the probes, can be digested away by addition of nuclease, leaving just the hybrid duplexes of target sequence of complementary probe.

It is generally recognized that nucleic acids are denatured by increasing the temperature and/or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus, specificity of hybridization is reduced at lower stringency. Conversely, at higher stringency (e.g., higher temperature or lower salt) successful hybridization requires fewer mismatches. One of skill in the art will appreciate that hybridization conditions can be designed to provide different degrees of stringency. The strength of hybridization can be increased without lowering the stringency of hybridization, and thus the specificity of hybridization can be maintained in a high stringency buffer, by including unnatural bases in the probes, such as by including locked nucleic acids or peptide nucleic acids.

In general, there is a tradeoff between hybridization specificity (stringency) and signal intensity. Thus, in one embodiment, the wash is performed at the highest stringency that produces consistent results and that provides a signal intensity greater than approximately 10% of the background intensity. Thus, the hybridization complexes (e.g., as captured on an array surface) may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

Changes in expression of a nucleic and/or the presence of nucleic acid detected by these methods for instance can include increases or decreases in the level (amount) or functional activity of such nucleic acids, their expression or translation into protein, or in their localization or stability. An increase or a decrease, for example relative to a normalization biomarker, can be, for example, at least a 1-fold, at least a 2-fold, or at least a 5-fold, such as about a 1-fold, 2-fold, 3-fold, 4-fold, 5-fold, change (increase or decrease) in the expression of and/or the presence of a particular nucleic acid, such as a nucleic acid corresponding to the biomarker shown in any of Tables 2-6. In multiplexed method embodiments, the relative expression of non-normalizer genes (e.g., variable genes; for example, Tables 2-6) also can be compared; particularly, when each such gene has been similarly normalized (e.g., to the expression of one or more co-detected normalizer genes; for example see Table 7). Hence, the normalized expression of one variable gene may be at least at least a 1-fold, at least a 2-fold, or at least a 5-fold, such as about a 1-fold, 1.5-fold, 2-fold, 3-fold, 4-fold, 5-fold higher or lower than the normalized expression of another variable gene.

In one example, gene expression is measured using a multiplexed methodology. In such methods, a plurality of measurements (e.g., gene expression measurements) can be made in a single sample. Various technologies have evolved that permit the monitoring of large numbers of genes in a single sample (e.g., traditional microarrays, multiplexed PCR, serial analysis of gene expression (SAGE; e.g., U.S. Pat. No. 5,866,330), multiplex ligation-dependent probe amplification (MLPA), high-throughput sequencing, labeled bead-based technology (e.g., U.S. Pat. Nos. 5,736,330 and 6,449,562), digital molecular barcoding technology (e.g, U.S. Pat. No. 7,473,767).

Arrays are one particularly useful (non-limiting) set of tools for multiplex detection of gene expression. An array is a systematic arrangement of elements (e.g., analyte capture reagents (such as, target-specific oligonucleotide probes, aptamers, or antibodies)) where a set of values (e.g., gene expression values) can be associated with an identification key. The arrayed elements may be systematically identified on a single surface (e.g., by spatial mapping or by differential tagging), using separately identifiable surfaces (e.g., flow channels or beads), or by a combination thereof.

Other useful embodiments involve high-throughput methodology, with which multiple samples may be queried at one time. High-throughput, multiplexed embodiments (contemporaneously measuring the expression of a plurality of genes in a plurality of samples) also are contemplated. Examples of methods and assay systems that can be used to detect the disclosed biomarkers are high throughput assay techniques disclosed in International Patent Publication Nos. WO 2003/002750 and WO 2008/121927, WO 1999/032663, WO 2000/079008, WO/2000/037684, and WO 2000/037683 and U.S. Pat. Nos. 6,232,066, 6,458,533, 6,238,869, and 7,659,063, which are incorporated by reference herein in so far as they describe high throughput assay techniques.

In some array embodiments, nucleic acid sequences of interest (such as oligonucleotides) that are designed to capture (directly or indirectly) one or more products of the genes shown in Tables 2-7 are plated or arrayed on a microchip substrate. For example, the array can include oligonucleotides complementary to at least two of the genes shown in Table 3 (such as at least 3, at least 5, at least 10, at least 20, or all 28 of the genes shown in Table 3 and optionally, at least one of the genes shown in Table 7). In other examples, the array can include oligonucleotides complementary to a portion of a nuclease protection probe that is complementary to a product of at least two of the genes shown in Table 3 (such as at least 3, at least 5, at least 10, at least 20, or all 28 of the genes shown in Table 3), and optionally, to at least one of the genes shown in Table 7). In one example, the array can include oligonucleotides complementary to at least two of the genes shown in Table 4 (such as at least 3, at least 4, at least 5, at least 6, at least 7, or all 8 of the genes shown in Table 4 and optionally, at least one of the genes shown in Table 7). In other examples, the array can include oligonucleotides complementary to a portion of a nuclease protection probe that is complementary to a product of at least two of the genes shown in Table 4 (such as at least 3, at least 4, at least 5, at least 6, at least 7, or all 8 of the genes shown in Table 4), and optionally, to at least one of the genes shown in Table 7). In one example, the array can include oligonucleotides complementary to at least one gene shown in Table 5 (such as at least 2, at least 3, at least 5, at least 10, at least 15, or all 17 of the genes shown in Table 5 and optionally, at least one of the genes shown in Table 7). In other examples, the array can include oligonucleotides complementary to a portion of a nuclease protection probe that is complementary to a product of at least one gene shown in Table 5 (such as at least 2, at least 3, at least 5, at least 10, at least 15, or all 17 of the genes shown in Table 5), and optionally, to at least one of the genes shown in Table 7). In one example, the array can include oligonucleotides complementary to at least one gene shown in Table 6 (such as 1, 2, 3, 4, 5, or all 6 of the genes shown in Table 6 and optionally, at least one of the genes shown in Table 7). In other examples, the array can include oligonucleotides complementary to a portion of a nuclease protection probe that is complementary to a product of at least one gene shown in Table 6 (such as 1, 2, 3, 4, 5, or all 6 of the genes shown in Table 6), and optionally, to at least one of the genes shown in Table 7).

The arrayed sequences are then hybridized with isolated nucleic acids, such as cDNA or RNA (e.g., mRNA, miRNA and/or lncRNA), from the test sample (e.g., lung sample obtained from a subject, whose characterization as squamous or nonsquamous NSCLC is desired). In one example, the isolated nucleic acids from the test sample are labeled, such that their hybridization with the specific complementary oligonucleotide on the array can be determined. Alternatively, the test sample nucleic acids are not labeled, and hybridization between the oligonucleotides on the array and the target nucleic acid is detected using a sandwich assay, for example using additional oligonucleotides complementary to the target that are labeled.

In one embodiment, the hybridized nucleic acids are detected by detecting one or more labels attached to the sample nucleic acids or attached to a nucleic acid probe that hybridizes directly or indirectly to the target nucleic acids. The labels can be incorporated by any of a number of methods. In one example, the label is simultaneously incorporated during the amplification step in the preparation of the sample nucleic acids. Thus, for example, polymerase chain reaction (PCR) with labeled primers or labeled nucleotides will provide a labeled amplification product. In one embodiment, transcription amplification using a labeled nucleotide (such as fluorescein-labeled UTP and/or CTP) incorporates a label into the transcribed nucleic acids.

Detectable labels suitable for use include any composition detectable by spectroscopic, photochemical, biochemical, immunochemical, electrical, optical or chemical means. Useful labels include biotin for staining with labeled streptavidin conjugate, magnetic beads (for example DYNABEADS™), fluorescent dyes (for example, fluorescein, Texas red, rhodamine, green fluorescent protein, and the like), radiolabels (for example, ³H, ¹²⁵I, ³⁵S, ¹⁴C, or ³²P), enzymes (for example, horseradish peroxidase, alkaline phosphatase and others commonly used in an ELISA), and colorimetric labels such as colloidal gold or colored glass or plastic (for example, polystyrene, polypropylene, latex, etc.) beads. Patents teaching the use of such labels include U.S. Pat. No. 3,817,837; U.S. Pat. No. 3,850,752; U.S. Pat. No. 3,939,350; U.S. Pat. No. 3,996,345; U.S. Pat. No. 4,277,437; U.S. Pat. No. 4,275,149; and U.S. Pat. No. 4,366,241.

Means of detecting such labels are also well known. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

The label may be added to the target (sample) nucleic acid(s) prior to, or after, the hybridization. So-called “direct labels” are detectable labels that are directly attached to or incorporated into the target (sample) nucleic acid prior to hybridization. In contrast, so-called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected (see Laboratory Techniques in Biochemistry and Molecular Biology, Vol. 24: Hybridization With Nucleic Acid Probes, P. Tijssen, ed. Elsevier, N. Y., 1993).

In situ hybridization (ISH), such as s chromogenic in situ hybridization (CISH) and silver in situ hybridization (SISH), is an exemplary method for detecting and comparing expression of genes of interest (such as those in Tables 2-8). ISH applies and extrapolates the technology of nucleic acid hybridization to the single cell level, and, in combination with the art of cytochemistry, immunocytochemistry and immunohistochemistry, permits the maintenance of morphology and the identification of cellular markers to be maintained and identified, and allows the localization of sequences to specific cells within populations, such as tissue samples. ISH is a type of hybridization that uses a complementary nucleic acid to localize one or more specific nucleic acid sequences in a portion or section of tissue (in situ), or, if the tissue is small enough, in the entire tissue (whole mount ISH). RNA ISH can be used to assay expression patterns in a tissue, such as the expression of the biomarkers in any of Tables 2-8. DNA ISH (such as CISH and SISH) can be used to detect nucleic acids at the genomic level.

Sample cells or tissues are treated to increase their permeability to allow a probe, such as a probe specific for one or more of the biomarkers in any of Tables 2-8, to enter the cells. The probe is added to the treated cells, allowed to hybridize at pertinent temperature, and excess probe is washed away. A complementary probe may be labeled with a detectable label, such as a radioactive, fluorescent or antigenic tag, so that the probe's location and quantity in the tissue can be determined, for example using autoradiography, fluorescence microscopy or immunoassay.

In situ PCR is the PCR-based amplification of the target nucleic acid sequences prior to ISH. For detection of RNA, an intracellular reverse transcription step is introduced to generate complementary DNA from RNA templates prior to in situ PCR. This enables detection of low copy RNA sequences. Prior to in situ PCR, cells or tissue samples are fixed and permeabilized to preserve morphology and permit access of the PCR reagents to the intracellular sequences to be amplified. PCR amplification of target sequences is next performed either in intact cells held in suspension or directly in cytocentrifuge preparations or tissue sections on glass slides. In the former approach, fixed cells suspended in the PCR reaction mixture are thermally cycled using conventional thermal cyclers. After PCR, the cells are cytocentrifuged onto glass slides with visualization of intracellular PCR products by ISH or immunohistochemistry. In situ PCR on glass slides is performed by overlaying the samples with the PCR mixture under a coverslip which is then sealed to prevent evaporation of the reaction mixture. Thermal cycling is achieved by placing the glass slides either directly on top of the heating block of a conventional or specially designed thermal cycler or by using thermal cycling ovens.

Detection of intracellular PCR products is generally achieved by one of two different techniques, indirect in situ PCR by ISH with PCR-product specific probes, or direct in situ PCR without ISH through direct detection of labeled nucleotides (such as digoxigenin-11-dUTP, fluorescein-dUTP, 3H-CTP or biotin-16-dUTP), which have been incorporated into the PCR products during thermal cycling.

Quantitative Nuclease Protection Assay (qNPA)

In particular embodiments of the disclosed methods, the nucleic acid is detected in the sample utilizing a quantitative nuclease protection assay and array (such as an array described below). The quantitative nuclease protection assay is described in International Patent Publications WO 99/032663; WO 00/037683; WO 00/037684; WO 00/079008; WO 03/002750; and WO 08/121927; and U.S. Pat. Nos. 6,238,869; 6,458,533; and 7,659,063, each of which is incorporated herein by reference in their entirety. See also, Martel et al, Assay and Drug Development Technologies. 2002, 1 (1-1):61-71; Martel et al, Progress in Biomedical Optics and Imaging, 2002, 3:35-43; Martel et al, Gene Cloning and Expression Technologies, Q. Lu and M. Weiner, Eds., Eaton Publishing, Natick (2002); Seligmann, B. PharmacoGenomics, 2003, 3:36-43; Martel et al, “Array Formats” in “Microarray Technologies and Applications,” U. R. Muller and D. Nicolau, Eds, Springer-Verlag, Heidelberg; Sawada et al, Toxicology in Vitro, 20:1506-1513; Bakir et al., Biorg. & Med. Chem Lett, 17: 3473-3479; Kris, et al, Plant Physiol. 144: 1256-1266; Roberts et al., Laboratory Investigation, 87: 979-997; Rimsza et al., Blood, 2008 Oct. 15, 112 (8): 3425-3433; Pechhold et al., Nature Biotechnology, 27, 1038-1042. All of these are fully incorporated by reference herein.

Using qNPA methods, a nuclease protection probe (NPP) is allowed to hybridize to the target sequence, which is followed by incubation of the sample with a nuclease that digests single stranded nucleic acid molecules. Thus, if the probe is detected, (e.g. it is not digested by the nuclease) then the target of the probe, for example a target nucleic acid shown in any of Tables 2-8, is present in the sample, and this presence can be quantified. NPPs can be designed for individual targets and added to an assay as a cocktail for identification on an array. Thus multiple genes targets can be measured within the same assay and/or array.

In some examples, samples (e.g., cells or tissue) from the lung are first lysed or permeabilized in an aqueous solution (for example using a lysis buffer). The aqueous solution or lysis buffer includes detergent (such as sodium dodecyl sulfate) and one or more chaotropic agents (such as formamide, guanidinium HCl, guanidinium isothiocyanate, or urea). The solution may also contain a buffer (for example SSC). In some examples, the lysis buffer includes about 15% to 25% formamide (v/v), about 0.01% to 0.1% SDS, and about 0.5-6×SSC. The buffer may optionally include tRNA (for example, about 0.001 to about 2.0 mg/ml) or a ribonuclease. The lysis buffer may also include a pH indicator, such as Phenol Red. In a particular example, the lysis buffer includes 20% formamide, 3×SSC (79.5%), 0.05% DSD, 1 μg/ml tRNA, and 1 mg/ml Phenol Red. Cells are incubated in the aqueous solution for a sufficient period of time (such as about 1 minute to about 60 minutes, for example about 5 minutes to about 20 minutes, or about 10 minutes) and at a sufficient temperature (such as about 22° C. to about 115° C., for example, about 37° C. to about 105° C., or about 90° C. to about 110° C.) to lyse or permeabilize the cell. In some examples, lysis is performed at about 95° C., if the nucleic acid to be detected is RNA. In other examples, lysis is performed at about 105° C., if the nucleic acid to be detected is DNA.

In some examples, a nucleic acid protection probe (NPP) (such as those shown in SEQ ID NOS: 1-47) complementary to the target can be added to a sample at a concentration ranging from about 10 pM to about 10 nM (such as about 30 pM to 5 nM, about 100 pM to about 1 nM), in a buffer such as, for example, 6×SSPE-T (0.9 M NaCl, 60 mM NaH₂PO₄, 6 mM EDTA, and 0.05% Triton X-100) or lysis buffer (described above). In one example, the probe is added to the sample at a final concentration of about 30 pM. In another example, the probe is added to the sample at a final concentration of about 167 pM. In a further example, the probe is added to the sample at a final concentration of about 1 nM. In such examples, NPPs not digested by a nuclease, such as S1, if the NPP is hybridized to (forms a duplex with) a complementary sequence, such as a target sequence.

One of skill in the art can identify conditions sufficient for an NPP to specifically hybridize to its target present in the test sample. For example, one of skill in the art can determine experimentally the features (such as length, base composition, and degree of complementarity) that will enable a nucleic acid (e.g., fusion probe) to hybridize to another nucleic acid (e.g., a target nucleic acid in any of Tables 2-8) under conditions of selected stringency, while minimizing non-specific hybridization to other substances or molecules. Typically, the nucleic acid sequence of an NPP will have sufficient complementarity to the corresponding target sequence to enable it to hybridize under selected stringent hybridization conditions, for example hybridization at about 37° C. or higher (such as about 37° C., 42° C., 50° C., 55° C., 60° C., 65° C., 70° C., 75° C., or higher). Among the hybridization reaction parameters which can be varied are salt concentration, buffer, pH, temperature, time of incubation, amount and type of denaturant such as formamide.

The nucleic acids in the sample are denatured (for example at about 95° C. to about 105° C. for about 5-15 minutes) and hybridized to a NPP for between about 10 minutes and about 24 hours (for example, at least about 1 hour to 20 hours, or about 6 hours to 16 hours) at a temperature ranging from about 4° C. to about 70° C. (for example, about 37° C. to about 65° C., about 45° C. to about 60° C., or about 50° C. to about 60° C.). In some examples, the probes are incubated with the sample at a temperature of at least about 40° C., at least about 45° C., at least about 50° C., at least about 55° C., at least about 60° C., at least about 65° C., or at least about 70° C. In one example, the probes are incubated with the sample at about 60° C. In another example, the NPPs are incubated with the sample at about 50° C. These hybridization temperatures are exemplary, and one of skill in the art can select appropriate hybridization temperature depending on factors such as the length and nucleotide composition of the NPPs.

In some embodiments, the methods do not include nucleic acid purification (for example, nucleic acid purification is not performed prior to contacting the sample with the probes and/or nucleic acid purification is not performed following contacting the sample with the probes). In some examples, no pre-processing of the sample is required except for cell lysis. In some examples, cell lysis and contacting the sample with the NPPs occur sequentially, in some non-limiting examples without any intervening steps. In other examples, cell lysis and contacting the sample with the NPPs occur concurrently.

Following hybridization of the one or more NPPs and nucleic acids in the sample, the sample is subjected to a nuclease protection procedure. NPPs which have hybridized to a full-length nucleic acid are not hydrolyzed by the nuclease and can be subsequently detected.

Treatment with one or more nucleases will destroy nucleic acid molecules other than the probes which have hybridized to nucleic acid molecules present in the sample. For example, if the sample includes a cellular extract or lysate, unwanted nucleic acids, such as genomic DNA, cDNA, tRNA, rRNA and mRNAs other than the gene of interest, can be substantially destroyed in this step. One of skill in the art can select an appropriate nuclease, for example based on whether DNA or RNA is to be detected. Any of a variety of nucleases can be used, including, pancreatic RNAse, mung bean nuclease, S1 nuclease, RNAse A, Ribonuclease T1, Exonuclease III, Exonuclease VII, RNAse CLB, RNAse PhyM, RNAse U2, or the like, depending on the nature of the hybridized complexes and of the undesirable nucleic acids present in the sample. In a particular example, the nuclease is specific for single-stranded nucleic acids, for example S1 nuclease. An advantage of using a nuclease specific for single-stranded nucleic acids in some method embodiments disclosed here is to remove such single-stranded (“sticky”) molecules from subsequent reaction steps where they may lead to unnecessary background or cross-reactivity. S1 nuclease is commercially available from for example, Promega, Madison, Wis. (cat. no. M5761); Life Technologies/Invitrogen, Carlsbad, Calif. (cat. no. 18001-016); Fermentas, Glen Burnie, Md. (cat. no. EN0321), and others. Reaction conditions for these enzymes are well-known in the art and can be optimized empirically.

In some examples, S1 nuclease diluted in an appropriate buffer (such as a buffer including sodium acetate, sodium chloride, zinc sulfate, and detergent, for example, 0.25 M sodium acetate, pH 4.5, 1.4 M NaCl, 0.0225 M ZnSO₄, 0.05% KATHON) is added to the hybridized probe mixture and incubated at about 50° C. for about 30-120 minutes (for example, about 60-90 minutes) to digest non-hybridized nucleic acid and unbound NPP.

The samples optionally are treated to otherwise remove non-hybridized material and/or to inactivate or remove residual enzymes (e.g., by phenol extraction, precipitation, column filtration, etc.). In some examples, the samples are optionally treated to dissociate the target nucleic acid from the probe (e.g., using base hydrolysis and heat). After hybridization, the hybridized target can be degraded, e.g., by nucleases or by chemical treatments, leaving the NPPs in direct proportion to how much NPP had been hybridized to target. Alternatively, the sample can be treated so as to leave the (single strand) hybridized portion of the target, or the duplex formed by the hybridized target and the probe, to be further analyzed.

The presence of the NPPs (or the remaining target or target:NPP complex) is then detected. Any suitable method can be used to detect the probes (or the remaining target or target:NPP complex). In some examples, the NPPs include a detectable label and detecting the presence of the NPP(s) includes detecting the detectable label. In some examples, the NPPs are labeled with the same detectable label. In other examples, the NPPs are labeled with different detectable labels (such as a different label for each target). In other examples, the NPPs are detected indirectly, for example by hybridization with a labeled nucleic acid. In some examples, the NPPs are detected using a microarray, for example, a microarray including detectably labeled nucleic acids (for example labeled with biotin or horseradish peroxidase) that are complementary to the NPPs. In other examples, the NPPs are detected using a microarray including capture probes and programming linkers, wherein a portion of the programming linker is complementary to a portion of the NPPs and subsequently incubating with detection linkers, a portion of which is complementary to a separate portion of the NPPs. The detection linkers can be detectably labeled, or a separate portion of the detection linkers are complementary to additional nucleic acids including a detectable label (such as biotin or horseradish peroxidase).

In some examples, the NPPs are detected on a microarray, for example, as described in International Patent Publications WO 99/032663; WO 00/037683; WO 00/037684; WO 00/079008; WO 03/002750; and WO 08/121927; and U.S. Pat. Nos. 6,238,869; 6,458,533; and 7,659,063, incorporated herein by reference in their entirety. See also, Martel et al, Assay and Drug Development Technologies. 2002, 1 (1-1):61-71; Martel et al, Progress in Biomedical Optics and Imaging, 2002, 3:35-43; Martel et al, Gene Cloning and Expression Technologies, Q. Lu and M. Weiner, Eds., Eaton Publishing, Natick (2002); Seligmann, B. PharmacoGenomics, 2003, 3:36-43; Martel et al, “Array Formats” in “Microarray Technologies and Applications,” U. R. Muller and D. Nicolau, Eds, Springer-Verlag, Heidelberg; Sawada et al, Toxicology in Vitro, 20:1506-1513; Bakir, et al, Biorg. & Med. Chem Lett, 17: 3473-3479; Kris, et al, Plant Physiol. 144: 1256-1266; Roberts, et al, Laboratory Investigation, 87: 979-997; Rimsza, et al, Blood, 2008 Oct. 15, 112 (8): 3425-3433; Pechhold, et al, Nature Biotechnology, 27, 1038-1042.

Briefly, in one non-limiting example, following hybridization and nuclease treatment, the solution is neutralized and transferred onto a programmed ARRAYPLATE (HTG Molecular Diagnostics, Tucson, Ariz.; each element of the ARRAYPLATE is programmed to capture a specific probe, for example utilizing an anchor attached to the plate and a programming linker associated with the anchor), and the NPPs are captured during an incubation (for example, overnight at about 50° C.). The platform can instead be a NIMBLEGEN microarray (Roche Nimblegen, Madison, Wis.) or the probes can be captured on X-MAP beads (Luminex, Austin, Tex.), an assay referred to as the QBEAD assay, or processed further, including as desired PCR amplification or ligation reactions, and for instance then measured by sequencing). The media is removed and a cocktail of probe-specific detection linkers are added, in the case of the ARRAYPLATE and QBEAD assays, which hybridize to their respective (captured) probes during an incubation (for example, 1 hour at about 50° C.). This step is skipped in the case of the NIMBLEGEN microarray assays in circumstances where the probes are directly biotinylated and there is no use of detection linker. Specific for the ARRAYPLATE and QBEAD assays, the array or beads are washed and then a triple biotin linker (an oligonucleotide that hybridizes to a common sequence on every detection linker, with three biotins incorporated into it) is added and incubated (for example, 1 hour at about 50° C.). For certain ARRAYPLATE embodiments, HRP-labeled avidin (avidin-HRP) is added and incubated (for example at about 37° C. for 1 hour), then washed to remove unbound avidin-HRP. Substrate is added and the plate is imaged to measure the intensity of every element within the plate. In some QBEAD embodiments, avidin-PE is added, the beads are washed, and then measured by flow cytometry using the Luminex 200, FLEXMAP 3D, or other appropriate instrument. In the case of some NIMBLEGEN array embodiments, after the addition of avidin-HRP a tyramide signal amplification step is optionally carried out in the presence of substrate, resulting in the deposition of Cy3 labeled probe, the slides are washed, dried, and scanned in a standard microarray scanner. One of skill in the art can design suitable capture probes, programming linkers, detection linkers, and other reagents for use in a quantitative nuclease protection assay based upon the NPPs utilized in the methods disclosed herein.

Nucleic Acid Amplification

In some examples, nucleic acid molecules (such as nucleic acid gene products (e.g., mRNA or lncRNA) or nuclease protection probes) are amplified prior to or as a means to their detection. In some examples, nucleic acid expression levels are determined during amplification, for example by using real time RT-PCR.

In one example, a nucleic acid sample can be amplified prior to hybridization, for example hybridization to complementary oligonucleotides present on an array. If a quantitative result is desired, a method is utilized that maintains or controls for the relative frequencies of the amplified nucleic acids. Methods of “quantitative” amplification are well known. For example, quantitative PCR involves simultaneously co-amplifying a known quantity of a control sequence using the same primers. This provides an internal standard that can be used to calibrate the PCR reaction. The array can then include probes specific to the internal standard for quantification of the amplified nucleic acid.

In some examples, the primers used for the amplification are selected so as to amplify a unique segment of the gene product of interest (such as RNA of a gene shown in any of Tables 2-8). In other embodiments, the primers used for the amplification are selected so as to amplify a NPP specific for a gene product of interest (such as RNA of a gene shown in any of Tables 2-8). Primers that can be used to amplify variable gene products (e.g., shown in any of Tables 2-6), as well as normalization gene products (e.g., see Table 7), are commercially available or can be designed and synthesized according to well-known methods.

In one example, RT-PCR can be used to detect RNA (e.g., mRNA or lncRNA) levels in normal and lung tissue samples. Generally, the first step in gene expression profiling by RT-PCR is the reverse transcription of the RNA template into cDNA, followed by its exponential amplification in a PCR reaction. Two commonly used reverse transcriptases are avian myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp® RNA PCR kit (Perkin Elmer, CA), following the manufacturer's instructions. The derived cDNA can then be used as a template in the subsequent PCR reaction.

Although PCR can use a variety of thermostable DNA-dependent DNA polymerases, it typically employs the Taq DNA polymerase. TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendable by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments dissociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

A variation of RT-PCR is real time quantitative RT-PCR, which measures PCR product accumulation through a dual-labeled fluorogenic probe (e.g., Taqman® probe). Real time PCR is compatible both with quantitative competitive PCR, where internal competitor for each target sequence is used for normalization, and with quantitative comparative PCR using a normalization gene contained within the sample, or a normalization gene for RT-PCR (see Heid et al., Genome Research 6:986-994, 1996). Quantitative PCR is also described in U.S. Pat. No. 5,538,848. Related probes and quantitative amplification procedures are described in U.S. Pat. No. 5,716,784 and U.S. Pat. No. 5,723,591. Instruments for carrying out quantitative PCR in microtiter plates are available, e.g., from PE Applied Biosystems (Foster City, Calif.).

An alternative quantitative nucleic acid amplification procedure is described in U.S. Pat. No. 5,219,727. In this method, the amount of a target sequence (e.g., the expression product of a gene listed in any of Tables 2-8) in a sample is determined by simultaneously amplifying the target sequence and an internal standard nucleic acid segment (e.g., the expression product of a gene listed in any of Tables 2-8). The amount of amplified nucleic acid from each segment is determined and compared to a standard curve to determine the amount of the target nucleic acid segment that was present in the sample prior to amplification.

RNA Sequencing

RNA sequencing provides another way to obtain multiplexed and, in some embodiments, high-throughput gene expression information. Numerous specific methods of RNA sequencing are known and/or being developed in the art (for one review, see Chu and Corey, Nuc. Acid Therapeutics, 22:271 (2012)). Whole-transcriptome sequencing and targeted RNA sequencing techniques each are available and are useful in the disclosed methods. Representative methods for sequencing-based gene expression analysis include serial analysis of gene expression (SAGE), gene expression analysis by massively parallel signature sequencing (MPSS), whole transcriptome shotgun sequencing (aka, WTSS or RNA-Seq), or nuclease-protection sequencing (aka, qNPS or NPSeq; see PCT Pub. No. WO2012/151111).

### Proteins for Detecting Gene Expression

In some embodiments of the disclosed methods, determining the level of gene expression in a lung sample includes detecting one or more proteins (for example by determining the relative or actual amounts of such proteins) in the sample. Routine methods of detecting proteins are known in the art, and the disclosure is not limited to particular methods of protein detection.

Protein gene products (e.g., those in any of Tables 2-8) or normalization proteins (e.g., those in Table 7) can be detected and the level of protein expression in the sample can be determined through novel epitopes recognized by protein-specific binding agents (such as antibodies or aptamers) specific for the target protein (such as those in any of Tables 2-8) used in immunoassays, such as ELISA assays, immunoblot assays, flow cytometric assays, immunohistochemical assays, an enzyme immunoassay, radioimmuno assays, Western blot assays, immunofluorescent assays, chemiluminescent assays and other peptide detection strategies (Wong et al., Cancer Res., 46: 6029-6033, 1986; Luwor et al., Cancer Res., 61: 5355-5361, 2001; Mishima et al., Cancer Res., 61: 5349-5354, 2001; Ijaz et al., J. Med. Virol., 63: 210-216, 2001). Generally these methods utilize monoclonal or polyclonal antibodies.

Thus, in some embodiments, the level of target protein expression (such as those in any of Tables 2-8) present in the biological sample and thus the amount of protein expressed is detected using a target protein specific binding agent, such as an antibody of fragment thereof, which can be detectably labeled. In some embodiments, the specific binding agent is an antibody, such as a polyclonal or monoclonal antibody, that specifically binds to the target protein (such as those in any of Tables 2-8). Thus in certain embodiments, determining the level or amount of protein in a biological sample includes contacting a sample from the subject with a protein specific binding agent (such as an antibody that specifically binds a protein shown in any of Tables 2-8), detecting whether the binding agent is bound by the sample, and thereby measuring the amount of protein present in the sample. In one embodiment, the specific binding agent is a monoclonal or polyclonal antibody that specifically binds to the target protein (such as those in any of Tables 2-8). One skilled in the art will appreciate that there are commercial sources for antibodies to target proteins, such as those in any of Tables 2-8.

The presence of a target protein (such as those in any of Tables 2-8) can be detected with multiple specific binding agents, such as one, two, three, or more specific binding agents. Thus, the methods can utilize more than one antibody. In some embodiments, one of the antibodies is attached to a solid support, such as a multiwell plate (such as, a microtiter plate), bead, membrane or the like. In practice, microtiter plates may conveniently be utilized as the solid phase. However, antibody reactions also can be conducted in a liquid phase.

In some examples, the method can include contacting the sample with a second antibody that specifically binds to the first antibody that specifically binds to the target protein (such as those in any of Tables 2-8). In some examples, the second antibody is detectably labeled, for example with a fluorophore (such as FITC, PE, a fluorescent protein, and the like), an enzyme (such as HRP), a radiolabel, or a nanoparticle (such as a gold particle or a semiconductor nanocrystal, such as a quantum dot (QDOT®)). In this method, an enzyme which is bound to the antibody will react with an appropriate substrate, such as a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods which employ a chromogenic substrate for the enzyme.

Detection can also be accomplished by visual comparison of the extent of enzymatic reaction of a substrate in comparison with similarly prepared standards. It is also possible to label the antibody with a fluorescent compound. Exemplary fluorescent labeling compounds include fluorescein isothiocyanate, rhodamine, phycoerythrin, phycocyanin, allophycocyanin, o-phthaldehyde, Cy3, Cy5, Cy7, tetramethylrhodamine isothiocyanate, phycoerythrin, allophycocyanins, Texas Red and fluorescamine. The antibody can also be detectably labeled using fluorescence emitting metals such as ¹⁵²Eu, or others of the lanthanide series. Other metal compounds that can be conjugated to the antibodies include, but are not limited to, ferritin, colloidal gold, such as colloidal superparamagnetic beads. These metals can be attached to the antibody using such metal chelating groups as diethylenetriaminepentacetic acid (DTPA) or ethylenediaminetetraacetic acid (EDTA). The antibody also can be detectably labeled by coupling it to a chemiluminescent compound. Examples of chemiluminescent labeling compounds are luminol, isoluminol, theromatic acridinium ester, imidazole, acridinium salt and oxalate ester. Likewise, a bioluminescent compound can be used to label the antibody. In one example, the antibody is labeled with a bioluminescence compound, such as luciferin, luciferase or aequorin. Haptens that can be conjugated to the antibodies include, but are not limited to, biotin, digoxigenin, oxazalone, and nitrophenol. Radioactive compounds that can be conjugated or incorporated into the antibodies include but are not limited to technetium 99m (⁹⁹Tc), ¹²⁵I and amino acids including any radionucleotides, including but not limited to, ¹⁴C, ³H and ³⁵S.

Generally, immunoassays for proteins (such as those in any of Tables 2-8) typically include incubating a biological sample in the presence of antibody, and detecting the bound antibody by any of a number of techniques well known in the art. In one example, the biological sample (such as one containing melanocytes) can be brought in contact with, and immobilized onto, a solid phase support or carrier such as nitrocellulose or a multiwell plate, or other solid support which is capable of immobilizing cells, cell particles or soluble proteins. The support may then be washed with suitable buffers followed by treatment with the antibody that specifically binds to the target protein (such as those in any of Tables 2-8). The solid phase support can then be washed with the buffer a second time to remove unbound antibody. If the antibody is directly labeled, the amount of bound label on solid support can then be detected by conventional means. If the antibody is unlabeled, a labeled second antibody, which detects that antibody that specifically binds to the target protein (such as those in any of Tables 2-8) can be used.

Alternatively, antibodies are immobilized to a solid support, and then contacted with proteins isolated from a biological sample, such as a tissue biopsy from the lung, under conditions that allow the antibody and the protein to bind specifically to one another. The resulting antibody: protein complex can then be detected, for example by adding another antibody specific for the protein (thus forming an antibody:protein:antibody sandwich). If the second antibody added is labeled, the complex can be detected, or alternatively, a labeled secondary antigay can be used that is specific for the second antibody added.

A solid phase support or carrier includes materials capable of binding a sample, antigen or an antibody. Exemplary supports include glass, polystyrene, polypropylene, polyethylene, dextran, nylon, amylases, natural and modified celluloses, polyacrylamides, gabbros and magnetite. The nature of the carrier can be either soluble to some extent or insoluble. The support material may have virtually any possible structural configuration so long as the coupled molecule is capable of binding to its target (such as an antibody or protein). Thus, the support configuration may be spherical, as in a bead, or cylindrical, as in the inside surface of a test tube, or the external surface of a rod. Alternatively, the surface may be flat such as a sheet or test strip.

In one embodiment, an enzyme linked immunosorbent assay (ELISA) is utilized to detect the target protein(s) (e.g., see Voller, “The Enzyme Linked Immunosorbent Assay (ELISA),” Diagnostic Horizons 2:1-7, 1978, Microbiological Associates Quarterly Publication, Walkersville, Md.; Voller et al., J. Clin. Pathol. 31:507-520, 1978; Butler, Meth. Enzymol. 73:482-523, 1981; Maggio, (ed.) Enzyme Immunoassay, CRC Press, Boca Raton, Fla., 1980; Ishikawa, et al., (eds.) Enzyme Immunoassay, Kgaku Shoin, Tokyo, 1981). ELISA can be used to detect the presence of a protein in a sample, for example by use of an antibody that specifically binds to a target protein (such as those in any of Tables 2-8). In some examples, the antibody can be linked to an enzyme, for example directly conjugated or through a secondary antibody, and a substance is added that the enzyme can convert to a detectable signal. Thus, in the case of fluorescence ELISA, when light of the appropriate wavelength is shone upon the sample, any antigen:antibody complexes will fluoresce so that the amount of antigen in the sample can be inferred through the magnitude of the fluorescence. The protein (such as proteins extracted or isolated from a melanocyte-containing sample) is usually immobilized on a solid support (for example polystyrene microtiter plate) either non-specifically (for example via adsorption to the surface) or specifically (for example via capture by another antibody specific to the same antigen, in a “sandwich” ELISA). Between each step the plate is typically washed with a mild detergent solution, such as phospho-buffered saline with or without NP40 or TWEEN to remove any proteins or antibodies that are not specifically bound. After the final wash step the plate is developed by adding an enzymatic substrate to produce a visible signal, which indicates the quantity of protein in the sample.

Detection can also be accomplished using any of a variety of other immunoassays. For example, by radioactively labeling the antibodies or antibody fragments, it is possible to detect fingerprint gene wild-type or mutant peptides through the use of a radioimmunoassay (RIA) (see, for example, Weintraub, B., Principles of Radioimmunoassays, Seventh Training Course on Radioligand Assay Techniques, The Endocrine Society, March, 1986). In another example, a sensitive and specific tandem immunoradiometric assay may be used (see Shen and Tai, J. Biol. Chem., 261:25, 11585-11591, 1986). The radioactive isotope can be detected by such means as the use of a gamma counter or a scintillation counter or by autoradiography.

In one example, a spectrometric method is utilized to detect or quantify an expression level of a target protein (such as those in any of Tables 2-8). Exemplary spectrometric methods include mass spectrometry, nuclear magnetic resonance spectrometry, and combinations thereof. In one example, mass spectrometry is used to detect the presence of a target protein (such as those in any of Tables 2-8) in a biological sample, such as a lung sample (see for example, Stemmann et al., Cell 107(6):715-26, 2001; Zhukov et al., “From Isolation to Identification: Using Surface Plasmon Resonance-Mass Spectrometry in Proteomics, PharmaGenomics, March/April 2002).

A target protein (such as those in any of Tables 2-8) also can be detected by mass spectrometry assays coupled to immunaffinity assays, the use of matrix-assisted laser desorption/ionization time-of-flight (MALDI-TOF) mass mapping and liquid chromatography/quadrupole time-of-flight electrospray ionization tandem mass spectrometry (LC/Q-TOF-ESI-MS/MS) sequence tag of proteins separated by two-dimensional polyacrylamide gel electrophoresis (2D-PAGE) (Kiernan et al., Anal. Biochem., 301: 49-56, 2002; Poutanen et al., Mass Spectrom., 15: 1685-1692, 2001).

Quantitative mass spectroscopic methods, such as SELDI, can be used to analyze protein expression in a sample (such as a lung sample). In one example, surface-enhanced laser desorption-ionization time-of-flight (SELDI-TOF) mass spectrometry is used to detect protein expression, for example by using the ProteinChip (Ciphergen Biosystems, Palo Alto, Calif.). Such methods are well known in the art (for example see U.S. Pat. No. 5,719,060; U.S. Pat. No. 6,897,072; and U.S. Pat. No. 6,881,586). SELDI is a solid phase method for desorption in which the analyte is presented to the energy stream on a surface that enhances analyte capture or desorption.

Briefly, one version of SELDI uses a chromatographic surface with a chemistry that selectively captures analytes of interest, such as those in any of Tables 2-8. Chromatographic surfaces can be composed of hydrophobic, hydrophilic, ion exchange, immobilized metal, or other chemistries. For example, the surface chemistry can include binding functionalities based on oxygen-dependent, carbon-dependent, sulfur-dependent, and/or nitrogen-dependent means of covalent or noncovalent immobilization of analytes. The activated surfaces are used to covalently immobilize specific “bait” molecules such as antibodies, receptors, or oligonucleotides often used for biomolecular interaction studies such as protein-protein and protein-DNA interactions.

The surface chemistry allows the bound analytes to be retained and unbound materials to be washed away. Subsequently, analytes bound to the surface (such as those in any of Tables 2-8) can be desorbed and analyzed by any of several means, for example using mass spectrometry. When the analyte is ionized in the process of desorption, such as in laser desorption/ionization mass spectrometry, the detector can be an ion detector. Mass spectrometers generally include means for determining the time-of-flight of desorbed ions. This information is converted to mass. However, one need not determine the mass of desorbed ions to resolve and detect them: the fact that ionized analytes strike the detector at different times provides detection and resolution of them. Alternatively, the analyte can be detectably labeled (for example with a fluorophore or radioactive isotope). In these cases, the detector can be a fluorescence or radioactivity detector. A plurality of detection means can be implemented in series to fully interrogate the analyte components and function associated with retained molecules at each location in the array.

Therefore, in a particular example, the chromatographic surface includes antibodies that specifically bind a target protein (such as those in any of Tables 2-8). In other examples, the chromatographic surface consists essentially of, or consists of, antibodies that specifically bind a target protein (such as those in any of Tables 2-8). In some examples, the chromatographic surface includes antibodies that bind other molecules, such as normalization proteins (e.g., those in any of Tables 2-8).

In another example, antibodies are immobilized onto the surface using a bacterial Fc binding support. The chromatographic surface is incubated with a sample, such as a sample of a nevus. The antigens present in the sample can recognize the antibodies on the chromatographic surface. The unbound proteins and mass spectrometric interfering compounds are washed away and the proteins that are retained on the chromatographic surface are analyzed and detected by SELDI-TOF. The MS profile from the sample can be then compared using differential protein expression mapping, whereby relative expression levels of proteins at specific molecular weights are compared by a variety of statistical techniques and bioinformatic software systems.

Alternatively, the amount of target protein can be determined using fluorescent methods. For example, quantum dots (e.g., Qdots®) are useful in a growing list of applications including immunohistochemistry, flow cytometry, and plate-based assays, and may therefore be used in conjunction with this disclosure. Quantum dot nanocrystals have unique optical properties including an extremely bright signal for sensitivity and quantitation; and high photostability for imaging and analysis. A single excitation source is needed, and a growing range of conjugates (e.g., antibody conjugates) makes them useful in a wide range of applications. The emission from quantum dots is narrow and symmetric, which means overlap with other colors is minimized, resulting in minimal bleed through into adjacent detection channels and attenuated crosstalk, in spite of the fact that many more colors can be used simultaneously. For example, IHC can be performed with quantum dot-conjugated secondary antibodies or streptavidin-conjugated quantum dots in combination with biotin-labeled primary or secondary antibodies.

### Optional Assay Control Measures

Optionally, assays used to detect gene expression products (e.g., nucleic acids (such as mRNA, lncRNA) or protein) will have both positive and negative process control elements used to assess assay performance.

A positive control can be any known element, preferably of a similar nature to the target (e.g., RNA target, then RNA (or cDNA) positive control), that can be included in an assay (or sample) and detected in parallel with the target(s) and that does not interfere (e.g., crossreact) with such target(s) detection. In one example, the positive control is an in vitro transcript (IVT) that is run in parallel as a separate sample or is “spiked” into each sample at a known amount. IVT-specific binding agents (e.g., oligonucleotide probes, such as a nuclease protection probe)) and, if applicable, IVT-specific detection agents also are included in each assay to ensure a positive result for such in vitro transcript. In another example, an IVT transcript can be designed from non-crossreacting regions of the Methanobacterium sp. AL-21 chromosome (NC_{—}015216).

Negative process control elements can include analyte-specific binding agents (e.g., oligonucleotides or antibodies) designed or selected to detect a gene product that is not expected to be expressed in the applicable test sample. For example, an analyte-specific binding agent that does recognize any gene expression product in the human transcriptome or proteome may be included in a multiplexed assay (such as an oligonucleotide probe or antibody specific for a plant or insect or nematode RNA or protein, respectively, where human gene expression products are the desired targets). This negative control element should not generate signal in the applicable assay. Any above-background signal for such negative process control element is an indicator of assay failure. In one example, the negative control is ANT.

Gene expression can vary across sample types or subjects due to the biology and/or due to variability related to specimen stability, integrity or input level as well as the assay process and system. In order to minimize non-biological related sources of variability (especially in multiplexed assays), gene expression products that do not or are found by bioinformatic methods not to significantly vary (e.g., “housekeepers” or normalizers) among samples of interest are measured in particular embodiments. In some such embodiments, expression levels for candidate normalization gene products will demonstrate adequate (e.g., above-background) and/or non-saturated intensity values. Further discussion of normalizer gene expression products is found elsewhere in this disclosure.

In some situations, anomalous signals may result from unexpected process-related issues that are not otherwise controlled, e.g., by analysis of normalizers; thus, in some embodiments, it is useful to include a sample-independent process control element(s) to indicate a successful or failed assay on any specimen, irrespective of the specimen stability, integrity, or input level. Method embodiments in which nucleic acid gene expression products are detected may include a known concentration of a RNA sample (e.g., in vitro transcript RNA or IVT) in every assay. Such a control element (e.g., IVT) will be measured in each assay and act as an assay process quality control.

The MAQC (Microarray Array Quality Control) project proposed that a “Universal Human Reference RNA” could be a useful external-control standard for microarray gene expression assays. Accordingly, some disclosed method embodiments involving RNA gene expression products may, but need not, include a parallel-processed sample containing Universal Human Reference RNA. If such universal RNA sample includes all or some of the RNAs targeted for detection by the applicable assay, a positive signal can be expected for such included RNAs, which may serve as an (or another) assay process quality control.

### Gene Expression Data

It is well accepted that gene expression data “contain the keys to address fundamental problems relating to the prevention and cure of diseases, biological evolution mechanisms and drug discovery” (Lu and Han, Information Systems, 28:243-268 (2003)). In some examples, distilling the information from such data is as simple as making a qualitative determination from the presence, absence or qualitative amount (e.g., high, medium, low) of one or more gene products detected. In other examples, raw gene expression data may be pre-processed (e.g., background subtracted, log transformed, and/or corrected), normalized, and/or applied in classification algorithms. These aspects are described in more detail below.

### Data Pre-Processing

Background Subtraction

In some method embodiments, raw gene expression data can be background subtracted. This correction is can be used, for example, where data has been collected using multiplexed methods, such as microarrays. One aim of such transformation is to correct for local effects, e.g., where one portion of a microarray surface may look “brighter” than another portion of the surface without any biological reason. Methods of background subtraction are well known in the art and include, e.g., (i) local background subtraction (e.g., consider all pixels that are outside the spot mask but within the bounding box centered at the spot center), (ii) morphological opening background estimation (relies on non-linear morphological filters, such as opening, erosion, dilation and rank filters (see, Soille, Morphological Image Analysis: Principles and Applications, Berlin: Springer-Verlag (1999), to create a background image for subtraction from the original image), (iii) constant background (subtracts a constant background for all spots), Normexp background correction (a convolution of normal and exponential, distributions is fitted to the foreground intensities, using the background intensities as a covariate, and the expected signal given the observed foreground becomes the corrected intensity).

Data Transformation

Many biological variables (e.g., gene expression data) do not meet the assumptions of parametric statistical tests, e.g., such variables are not normally distributed, the variances are not homogeneous, or both (Durbin et al., Bioinformatics, 18:S105 (2002). In some cases, transforming the data will make it fit the statistical assumptions better. In some method embodiments, useful data transformation can include (i) log transformation, which consists of taking the log of each observation, e.g., base-10 logs, base-2 logs, base-e logs (also known as natural logs); the log selection makes no difference because such logs differ by a constant factor; or variance-stabilizing transformation, e.g., as described by Durbin (supra).

Data Filters

Gene expression data may be filtered in some method embodiments to remove data that may be considered unreliable. It is understood that there are many methods known in the art for assessing the reliability of gene expression data and the following non-limiting examples are merely representative.

Gene expression data may be excluded from analysis, in some cases, if it is not expressed or is expressed at an undetectable level (not above background). Oppositely, gene expression data may be excluded from analysis, in some cases, if the expression of a negative control (e.g., ANT) gene is greater than an standard cut off (e.g., more than 100, 200, 250, or 300 relative light units, or more than 1%, 2%, 3%, 4%, 5%, 6%, 7%, 8%, 9%, or 10% above background).

For embodiments involving probe-sets or genes, there are a number of specific data filters that may be useful, including:
(i) Data arising from unreliable probe sets may be selected for exclusion from analysis by ranking probe-set reliability against a series of reference datasets. For example, RefSeq and Ensembl (EMBL) are considered very high quality reference datasets. Data from probe sets matching RefSeq or Ensembl sequences may in some cases be specifically included in microarray analysis experiments due to their expected high reliability. Similarly data from probe-sets matching less reliable reference datasets may be excluded from further analysis, or considered on a case by case basis for inclusion; or
(ii) Probe-sets that exhibit no, or low variance may be excluded from further analysis. Low-variance probe-sets are excluded from the analysis via a Chi-Square test. A probe-set is considered to be low-variance if its transformed variance is to the left of the 99 percent confidence interval of the Chi-Squared distribution with (N−1) degrees of freedom; or
(iii) Probe-sets for a given gene or transcript cluster may be excluded from further analysis if they contain less than a minimum number of probes, e.g., following other data pre-processing steps. For example in some embodiments, probe-sets for a given gene or transcript cluster may be excluded from further analysis if they contain less than 1, 2, 3, 4, or 5 probes.

Optionally, a statistical outlier program can be used that determines whether one of several replicates is statistically an outlier compared to the others, such as judged by being “x” standard deviations (SD) (e.g. at least 2-SD or at least 3-SD) away from the average, or CV % of replicates greater than a specified amount (e.g., at least 8% in log-transformed space). In an array-based assay, an outlier could result from there being a problem with one of the array spots, or due to an imaging artifact. Outlier removal is typically performed on a gene-by-gene basis, and if most of the genes in one replicate are outliers, one can apply a pre-established rule that eliminates the entire replicate. For instance, a pipetting error resulting in the improper addition of a critical reagent could cause the entire replicate to be an outlier.

In some examples where gene expression is measured in sample replicates (e.g., triplicates), reproducibility can be measured by pairwise correlation and by pairwise sample linear regression, and a correlation r >=0.95 used as acceptance of replicate (e.g., triplicate) reproducibility. In more specific examples, replicates with pairwise correlation r=>0.90 can be further reviewed by a simple regression model; in which case, if the intercept of the linear regression is statistically signicantly different from zero, the replicate removed from further consideration. Any sample with more than 25% (e.g., 1 out of 4) or more, 33% (e.g., 1 out of 3) or more, 50% (e.g., 2 out of 4) or more, or 67% (e.g., 2 out of 3) or more failed replicates may be considered a “failed sample” and removed from further analysis.

### Normalization

The objective of normalization is to remove variability due to experimental error (for example due to be due to pipetting, plate position, image artifacts, different amounts of total RNA, etc.) so that variation due to biological effects can be observed and quantified. This process helps ensure the differences observed between different sample types is due truly to difference in sample biology and not due to some technical artifact. There are several points during experimentation at which errors can be introduced and which can be eliminated by normalization.

In some embodiments, the expression of one or more “normalization biomarkers” can be determined or measured, such as one or more those in Table 7. For example, expression of 1, 2, 3, 4, 5, 6, 7, 8, 9, 10 or all of EEF2, DDX17, HMGXB3, RPL19, RPSA, RPS29, RPL37A, RPL41, CFL1, MTND4, or OAZ1 can be detected in the test sample. Disclosed methods can include normalizing raw expression values for each of the at least two biomarkers in Tables 2-6 (such as Table 3 or 4, or Table 5, or Table 6) to at least one normalization biomarker(s).

Alternatively, one or more normalization biomarkers useful in a disclosed method can be identified using the methods provided herein. For example, a normalization biomarker is any constitutively expressed gene (or protein) against whose expression another expressed gene (or protein) can be compared (e.g., by dividing (or subtracting, typically, after log transformation) the expression of one by the other). Accordingly, a normalization biomarker can be one or a plurality of genes or proteins, other than the biomarkers in Table 2-6, the expression of which does not significantly differ in a representative plurality of lung samples, such as squamous NSCLC, nonsquamous NSCLC, large cell lung cancer, small cell lung cancer, pulmonary carcinoids, and lung metastases of colon tumors. The distribution of expression values for the plurality of biomarkers whose expression was measured (such as biomarkers not listed in Table 2-6) can be determined and, optionally, outliers removed. The method can further include calculating a population central tendency (e.g., average, mean or median) expression value for the plurality of biomarkers (such as those not listed in Table 2-6), which central-tendency expression value is used for normalizing the raw expression values for each of the at least two biomarkers shown in Tables 2-6 (such as Table 2-4, or Table 5, or Table 6).

In other specific examples, the robust multi-array average (RMA) method may be used to normalize the raw data. The RMA method begins by computing background-corrected intensities for each matched cell on a number of microarrays. The background corrected values are restricted to positive values as described by Irizarry et al. (Biostatistics, 4:249 (2003)). After background correction, the base-2 logarithm of each background-corrected matched-cell intensity is then obtained. The background-corrected, log-transformed, matched intensity on each microarray is then normalized using the quantile normalization method in which for each input array and each probe expression value, the array percentile probe value is replaced with the average of all array percentile points, this method is more completely described by Bolstad et al. (Bioinformatics 19(2):185 (2003)). Following quantile normalization, the normalized data may then be fit to a linear model to obtain an expression measure for each probe on each microarray.

### Feature Selection (FS)

Classification algorithms typically perform suboptimally with thousands of features (genes/proteins). Thus, feature selection methods are used to identify features that are most predictive of a phenotype. The selected genes/proteins are presented to a classifier or a prediction model. The following benefits result from reducing the dimensionality of the feature space: (i) improve classification accuracy, (ii) provide a better understanding of the underlying concepts that generated the data, and (iii) overcome the risk of data overfitting, which arises when the number of features is large and the number of training patterns is comparatively small. Feature selection was used to determine the disclosed gene sets; therefore the corresponding classifiers have the foregoing advantages built in.

Feature selection techniques including filter techniques (which assess the relevance of features by looking at the intrinsic properties of the data), wrapper methods (which embed the model hypothesis within a feature subset search), and embedded techniques (in which the search for an optimal set of features is built into a classifier algorithm). Filter FS techniques useful in disclosed methods include: (i) parametric methods such as the use of two sample t-tests or moderated t-tests (e.g., LIMMA), ANOVA analyses, Bayesian frameworks, and Gamma distribution models, (ii) model free methods such as the use of Wilcoxon rank sum tests, between-within class sum of squares tests, rank products methods, random permutation methods, or total number of misclassifications (TNoM) which involves setting a threshold point for fold-change differences in expression between two datasets and then detecting the threshold point in each gene that minimizes the number of missclassifications, and (iii) multivariate methods such as bivariate methods, correlation based feature selection methods (CFS), minimum redundancy maximum relavance methods (MRMR), Markov blanket filter methods, tree-based methods, and uncorrelated shrunken centroid methods. Wrapper methods useful in disclosed methods include sequential search methods, genetic algorithms, and estimation of distribution algorithms. Embedded methods useful in the methods of the present disclosureinclude random forest algorithms, weight vector of support vector machine algorithms, and weights of logistic regression algorithms. Saeys et al. describe the relative merits of the filter techniques provided above for feature selection in gene expression analysis. In some embodiments, feature selection is provided by use of the LIMMA software package (Smyth, LIMMA: Linear Models for Microarray Data, In: Bioinformatics and Computational Biology Solutions, ed. by Gentleman et al., New York:Springer, pages 397-420 (2005)).

### Sample Type Confirmation

Disclosed herein are gene sets and classifiers that subtype NSCLC samples to squamous cell and nonsquamous cell NSCLC. Initial characterization (e.g., diagnosis) of NSCLC samples, typically, is done by histopathology and immunohistochemistry. Such samples may be misidentified. At least in the Examples performed in this disclosure, samples of colon origin in the lung (e.g., colon adenocarcinoma metastases) and small cell lung cancers and pulmonary carcinoids were found. These misidentified samples confound a NSCLC subtyping classifier.

Accordingly, some disclosed NSCLC squamous/nonsquamous gene sets or classifiers are benefitted by assurance that the input samples are, in fact, NSCLC samples. Accordingly, some method embodiments may further include the use of a pre-NSCLC classifier algorithm or gene set. For example, a pre-NSCLC classifier algorithm or gene set may use a tissue-type-specific molecular fingerprint (e.g., gene set or algorithm that identifies cells of colon origin in the lung, or that identifies non-NSCLC samples, such as small cell lung cancer and pulmonary carcinoids) to sort (or pre-classify) the samples according to their composition. Samples may be removed from further analysis because they are determined by a pre-NSCLC classifier algorithm or gene set not to be NSCLC, or pre-NSCLC classifier data/information may be incorporated in to a final classification algorithm which would incorporate that information (e.g., decision tree algorithm) to aid in the final NSCLC classifier output.

Gene sets and algorithms for colon-originating samples in the lung (see Table 5) and the group of small cell lung cancer and pulmonary carcinoids (see Table 6) are described in detail elsewhere in this disclosure and are useful for pre-NSCLC classifer sample-type indentification or confirmation.

### Classifier Algorithms

In some methods, gene expression information (e.g., for the biomarkers described in any of Tables 2-6, such as Tables 2-4) is applied to an algorithm in order to classify the expression profile (e.g., whether a NSCLC sample is squamous or nonsquamous subtype or neither (such as, indeterminant)). Disclosed herein are gene expression-based classifiers for the subtyping of NSCLC samples into squamous NSCLC and nonsquamous NSCLC. Specific classifier embodiments are described and, based on the provided gene sets and classification methods, others now are enabled.

A classifier is a predictive model (e.g., algorithm or set of rules) that can be used to classify test samples (e.g., NSCLC samples) into classes (or groups) (e.g., squamous NSCLC and nonsquamous NSCLC) based on the expression of genes in such samples (such as the genes in any of Tables 2-6, such as Tables 2-4). Unlike cluster analysis for which the number of clusters is unknown in advance, a classifier is trained on one or more sets of samples for which the desired class value(s) (e.g., squamous NSCLC and nonsquamous NSCLC) is (are) known. Once trained, the classifier is used to assign class value(s) to future observations. Typical classification algorithms, include: Centroid Classifiers, k Nearest Neighbors (kNN), Bayesian Classification (e.g., Naïve Bayes and Bayesian Networks), Decision Trees, Neural Networks, Regression Models, Linear Discriminant Analysis, and Support Vector Machines, each of which is contemplated by this disclosure, and some of which are described in more detail below.

Simplistically, a squamous/nonsquamous NSCLC classifier would be applied only to NSCLC samples; however, in practice, NSCLC samples often are misidentified by other methods. Accordingly, some disclosed classifiers (e.g., decision tree classifiers) also including rules for first identifying non-NSCLC samples, such as colon metastases in the lung and the group of small cell lung cancers and pulmonary carcinoids. Then, subsequent rules are used to assign the yet-unassigned samples to squamous NSCLC or nonsquamous NSCLC or neither (e.g., indeterminant) groupings. In some instances, members of the non-NSCLC groupings may be called “indeterminant” as well, in which examples, there are three outputs to the classifier: squamous NSCLC, nonsquamous NSCLC or neither (e.g., not determined or indeterminant or the like).

Illustrative algorithms include, but are not limited to, methods that reduce the number of variables such as principal component analysis algorithms, partial least squares methods, and independent component analysis algorithms. Illustrative algorithms further include, but are not limited to, methods that handle large numbers of variables directly such as statistical methods and methods based on machine learning techniques. Statistical methods include penalized logistic regression, prediction analysis of microarrays (PAM), methods based on shrunken centroids, support vector machine analysis, and regularized linear discriminant analysis. Machine learning techniques include bagging procedures, boosting procedures, random forest algorithms, and combinations thereof. Boulesteix et al. (Cancer Inform., 6:77 (2008)) provide an overview of the classification techniques provided above for the analysis of multiplexed gene expression data.

In some embodiments, results are classified using a trained algorithm. Trained algorithms of the present disclosure include algorithms that have been developed using a reference set of known squamous and nonsquamous NSCLC as well as, in some embodiments, large cell lung carcinoma, small cell lung carcinoma, colon metastastes in the lung, and pulmonary carcinoids, including but not limited to the sample types listed in Table 1. Algorithms suitable for categorization of samples include, but are not limited to, k-nearest neighbor algorithms, concept vector algorithms, naive bayesian algorithms, neural network algorithms, hidden markov model algorithms, genetic algorithms, and mutual information feature selection algorithms or any combination thereof. In some cases, trained algorithms of the present disclosure may incorporate data other than gene expression data such as but not limited to scoring or diagnosis by cytologists or pathologists of the present disclosure, information provided by a disclosed pre-classifier algorithm or gene set, or information about the medical history of a subject from whom a tested sample is taken.

In some specific embodiments, a support vector machine (SVM) algorithm, a random forest algorithm, or a combination thereof provides classification of samples (e.g., NSCLC samples) into squamous or nonsquamous NSCLC subtypes or identifies lung samples that are not NSCLC (such as, colon-originating lung samples or small cell lung cancer and pulmonary carcinoids). In some embodiments, identified markers that distinguish samples (e.g., lung versus colon or NSCLC versus not NSCLC) or distinguish subtypes (e.g., squamous and nonsquamous NSCLC)) are selected based on statistical significance. In some cases, the statistical significance selection is performed after applying a Benjamini Hochberg correction for false discovery rate (FDR) (see, J. Royal Statistical Society, Series B (Methodological) 57:289 (1995)).

In some cases, a disclosed classifier algorithm may be supplemented with a meta-analysis approach such as that described by Fishel et al. (Bioinformatics, 23:1599 (2007)). In some cases, the classifier algorithm may be supplemented with a meta-analysis approach such as a repeatability analysis. In some cases, the repeatability analysis selects markers that appear in at least one predictive expression product marker set.

### Exemplary Decision Tree Model

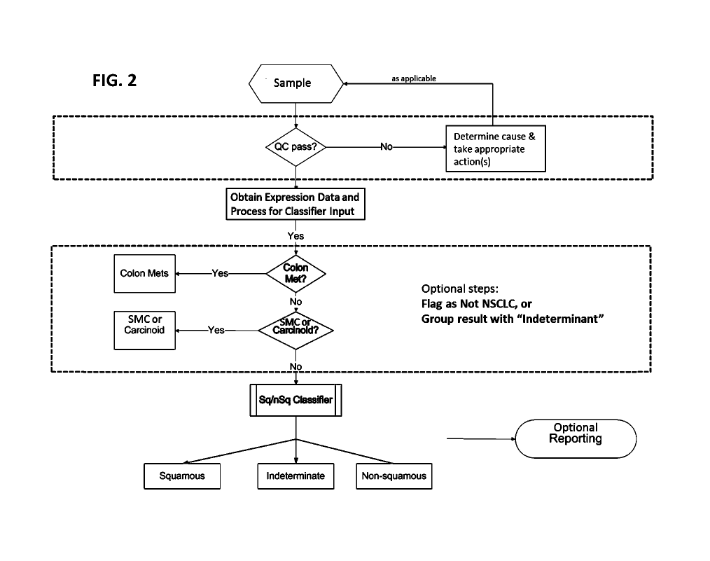

A decision tree algorithm is a flow-chart-like tree structure where each internal node denotes a test on an attribute, and a branch represents an outcome of the test. Leaf nodes represent class labels or class distribution. To generate a decision tree, all the training examples are used at the root, the logical test at the root of the tree is applied and training data then is partitioned into sub-groups based on the values of the logical test. This process is recursively applied (i.e., select attribute and split) and terminated when all the data elements in one branch are of the same class. To classify an unknown sample, its attribute values are tested against the decision tree. See, for example, all and parts of FIG. 2.

### Exemplary Logistic Regression Models

One representative method for developing statistical predictive models using the genes in any of Tables 2-6 is logistic regression with a binary distribution and a logit link function. Estimation for such models can be performed using Fischer Scoring. However, models estimated with exact logistic regression, Empirical Sandwich Estimators or other bias corrected, variance stabilized or otherwise corrective estimation techniques will also, under many circumstances, provide similar models which while yielding slightly different parameter estimates will yield qualitatively consistent patterns of results. Similarly, other link functions, including but not limited to a cumulative logit, complementary log-log, probit or cumulative probit may be expected to yield predictive models that give the same qualitative pattern of results.

One representative form of a predictive model (algorithm) is:

Logit(Yi)=β0+β1X1+β2X2+β3X3 . . . βnXn

where βo is an intercept term, βn is a coefficient estimate and Xn is the log base 2 expression value for a given gene. Typically, the value for all β will be greater than −1,000 and less than 1,000. Often, the β0 intercept term will be greater than −200 and less than 200 with cases in which it is greater than −100 and less than 100. The additional βn, where n>0, will likely be greater than −100 and less than 100.

Model performance may be validated with a number of tests known in the art, including, but not limited to, Wald Chi-Square test (overall model fit), and Hosmer and Lemeshow lack fit test (no statistically detectable lack of fit for the model). Predictors for each gene in the model should be stastically significant (e.g., p<0.05).

A number of cross validation methods are available to ensure reproducibility of the results. An exemplary method is a one-step maximum likelihood estimate approximation implemented as part of the SAS Proc Logistic classification table procedure. In some examples, ten (10)-fold cross validation and 66-33% split validation in the open source package Weka can be used for confirmation of results. In other examples, n-fold, including leave-one-out (LOO), cross validation and split sample training/testing provides useful confirmation of results.

The algorithms (aka, fitted model) provide a predicted event probability, which, for example, is the probability of a lung (e.g., NSCLC) sample being a squamous NSCLC. In some instances, a SAS computation method known to those of ordinary skill in the art can be used to compute a reduced-bias estimate of the predicted probability (see, support.sas.com/documentation/cdl/en/statug/63347/HTML/default/viewer.htm#statug_logistic_sec t044.htm (as of Mar. 15, 2013)). In other examples, a series of threshold values, z, where z is between 0 and 1 are set, as typically determined by the ordinarily skilled artisan based on the desired clinical utility of a model or application requirement. If the predicted probability calculated for a particular sample exceeds or equals the pre-set threshold value, z, the sample is assigned to the squamous NSCLC group; otherwise, it was assigned to the nonsquamous NSCLC group. In other examples, it two threshold values can be set where sample values falling between the two thresholds are assigned an “indeterminant” or “not otherwise assigned” or the like label.

### Molecular Profiling and Classifier Outputs

When classifying a biological sample, such as a lung sample, there are typically four possible outcomes from a binary classifier. If the outcome from a prediction is p and the actual value is also p, then it is called a true positive (TP); however if the actual value is n then it is said to be a false positive (FP). Conversely, a true negative has occurred when both the prediction outcome and the actual value are n, and false negative is when the prediction outcome is n while the actual value is p. Consider an embodiment that seeks to determine whether a sample is a squamous NSCLC. A false positive in this case occurs when a sample tests positive, but is not actually a squamous NSCLC. A false negative, on the other hand, occurs when the sample tests negative (i.e., not NSCLC), when it actually is a NSCLC sample. In some embodiments, ROC curve assuming real-world prevalence of subtypes can be generated by re-sampling errors achieved on available samples in relevant proportions.

The positive predictive value (PPV), or precision rate, or post-test probability of squamous cell NSCLC, is the proportion of samples with positive test results that correctly are squamous cell NSCLC. PPV reflects the probability that a positive test reflects the underlying hypothesis being tested (e.g., a sample is a squamous cell NSCLC). In one example:

False positive rate(α)=FP/(FP+TN)-specificity

False negative rate(β)=FN/(TP+FN)-sensitivity

Power=sensitivity=1−β

Likelihood-ratio positive=sensitivity/(1-specificity)

Likelihood-ratio negative=(1-sensitivity)/specificity

where TN is true negative, FN is false negative and TP and FP are as defined above.

Negative predictive value (NPV) is the proportion of subjects or samples with a negative test result (e.g., nonsquamous NSCLC or indeterminant) who are correctly diagnosed or subtyped. A high NPV for a given test means that when the test yields a negative result, it is most likely correct in its assessment.

In some embodiments, the results of the gene expression analysis of the disclosed methods provide a statistical confidence level that a given diagnosis (e.g., NSCLC subtype) is correct. In some embodiments, such statistical confidence level is above 85%, 90%, 91%, 92%, 93%, 94%, 95%, 96%, 97%, 98%, 99% or 99.5%.

In one aspect of the present disclosure, samples that have been processed by another method (e.g., histopathology and/or immunocytochemistry) and diagnosed are, then, subjected to disclosed molecular profiling as a second diagnostic screen. This second diagnostic screen enables, at least: 1) a significant reduction of false positives and false negatives, 2) a determination of the underlying genetic, metabolic, or signaling pathways responsible for the resulting pathology, 3) the ability to assign a statistical probability to the accuracy of the diagnosis, 4) the ability to resolve ambiguous results, and 5) the ability to distinguish between subtypes of NSCLC.

In some embodiments, the biological sample is classified as squamous NSCLC or nonsquamous NSCLC with an accuracy of greater than 75%, 80%, 85%, 86%, 87%, 88%, 89%, 90%, 91%, 92%, 93%, 94%, 95%, 96%, 97%, 98%, 99%, or 99.5%. The term accuracy as used in the foregoing sentence includes specificity, sensitivity, positive predictive value, negative predictive value, and/or false discovery rate.

In other cases, receiver operator characteristic (ROC) analysis may be used to determine the optimal assay parameters to achieve a specific level of accuracy, specificity, positive predictive value, negative predictive value, and/or false discovery rate. A ROC curve is a graphical plot that illustrates the performance of a binary classifier system as its discrimination threshold is varied. It is created by plotting the fraction of true positives out of the positives (TPR=true positive rate) vs. the fraction of false positives out of the negatives (FPR=false positive rate) at various threshold settings.

### Implementation: Methods, Classifiers and Systems

The methods, classifiers and systems described herein can be implemented in numerous ways. Several representative non-limiting embodiments are described below.

In some embodiments the data analysis involves a computer or other device, machine or apparatus for application of the various algorithms described herein, which is particularly advantageous where a large number of gene expression data points are collected and processed. Other embodiments involve use of a communications infrastructure, for example the internet. Various forms of hardware, software, firmware, processors, or a combination thereof are useful to implement specific classifier and method embodiments. Software can be implemented as an application program tangibly embodied on a program storage device, or different portions of the software implemented in the user's computing environment (e.g., as an applet) and on the reviewer's computing environment, where the reviewer may be located at a remote site associated (e.g., at a service provider's facility).

For example, during or after data input by the user, portions of the data processing can be performed in the user-side computing environment. For example, the user-side computing environment can be programmed to provide for defined test codes to denote a likelihood “score,” where the score is transmitted as processed or partially processed responses to the reviewer's computing environment in the form of test code for subsequent execution of one or more algorithms to provide a results and/or generate a report in the reviewer's computing environment. The score can be a numerical score (representative of a numerical value) or a non-numerical score representative of a numerical value or range of numerical values (e.g., “A” representative of a 90-95% likelihood of an outcome).

The application program for executing the algorithms described herein may be uploaded to, and executed by, a machine comprising any suitable architecture. In general, the machine involves a computer platform having hardware such as one or more central processing units (CPU), a random access memory (RAM), and input/output (I/O) interface(s). The computer platform also includes an operating system and microinstruction code. The various processes and functions described herein may either be part of the microinstruction code or part of the application program (or a combination thereof) which is executed via the operating system. In addition, various other peripheral devices may be connected to the computer platform such as an additional data storage device and a printing device.

As a computer system, the system generally includes a processor unit. The processor unit operates to receive information, which can include test data (e.g., level of a response gene, level of a reference gene product(s); normalized level of a response gene; and may also include other data such as patient data. This information received can be stored at least temporarily in a database, and data analyzed to generate a report as described above.

Part or all of the input and output data can also be sent electronically; certain output data (e.g., reports) can be sent electronically or telephonically (e.g., by facsimile, using devices such as fax back). Exemplary output receiving devices can include a display element, a printer, a facsimile device and the like. Electronic forms of transmission and/or display can include email, interactive television, and the like. In one embodiment, all or a portion of the input data and/or all or a portion of the output data (e.g., usually at least the final report) are maintained on a web server for access, preferably confidential access, with typical browsers. The data may be accessed or sent to health professionals as desired. The input and output data, including all or a portion of the final report, can be used to populate a patient's medical record which may exist in a confidential database at the healthcare facility.

A system for use in the methods described herein generally includes at least one computer processor (e.g., where the method is carried out in its entirety at a single site) or at least two networked computer processors (e.g., where data is to be input by a user (also referred to herein as a “client”) and transmitted to a remote site to a second computer processor for analysis, where the first and second computer processors are connected by a network, e.g., via an intranet or internet). The system can also include a user component(s) for input; and a reviewer component(s) for review of data, generated reports, and manual intervention. Additional components of the system can include a server component(s); and a database(s) for storing data (e.g., as in a database of report elements, e.g., interpretive report elements, or a relational database (RDB) which can include data input by the user and data output. The computer processors can be processors that are typically found in personal desktop computers (e.g., IBM, Dell, Macintosh), portable computers, mainframes, minicomputers, or other computing devices.

The networked client/server architecture can be selected as desired, and can be, for example, a classic two or three tier client server model. A relational database management system (RDMS), either as part of an application server component or as a separate component (RDB machine) provides the interface to the database.

In one example, the architecture is provided as a database-centric client/server architecture, in which the client application generally requests services from the application server which makes requests to the database (or the database server) to populate the report with the various report elements as required, particularly the interpretive report elements, especially the interpretation text and alerts. The server(s) (e.g., either as part of the application server machine or a separate RDB/relational database machine) responds to the client's requests.

The input client components can be complete, stand-alone personal computers offering a full range of power and features to run applications. The client component usually operates under any desired operating system and includes a communication element (e.g., a modem or other hardware for connecting to a network), one or more input devices (e.g., a keyboard, mouse, keypad, or other device used to transfer information or commands), a storage element (e.g., a hard drive or other computer-readable, computer-writable storage medium), and a display element (e.g., a monitor, television, LCD, LED, or other display device that conveys information to the user). The user enters input commands into the computer processor through an input device. Generally, the user interface is a graphical user interface (GUI) written for web browser applications.

The server component(s) can be a personal computer, a minicomputer, or a mainframe and offers data management, information sharing between clients, network administration and security. The application and any databases used can be on the same or different servers.

Other computing arrangements for the client and server(s), including processing on a single machine such as a mainframe, a collection of machines, or other suitable configuration are contemplated. In general, the client and server machines work together to accomplish the processing of the present disclosure.

Where used, the database(s) is usually connected to the database server component and can be any device which will hold data. For example, the database can be any magnetic or optical storing device for a computer (e.g., CDROM, internal hard drive, tape drive). The database can be located remote to the server component (with access via a network, modem, etc.) or locally to the server component.

Where used in the system and methods, the database can be a relational database that is organized and accessed according to relationships between data items. The relational database is generally composed of a plurality of tables (entities). The rows of a table represent records (collections of information about separate items) and the columns represent fields (particular attributes of a record). In its simplest conception, the relational database is a collection of data entries that “relate” to each other through at least one common field.

Additional workstations equipped with computers and printers may be used at point of service to enter data and, in some embodiments, generate appropriate reports, if desired. The computer(s) can have a shortcut (e.g., on the desktop) to launch the application to facilitate initiation of data entry, transmission, analysis, report receipt, etc. as desired.

### Computer-Readable Storage Media

The present disclosure also contemplates a computer-readable storage medium (e.g. CD-ROM, memory key, flash memory card, diskette, etc.) having stored thereon a program which, when executed in a computing environment, provides for implementation of algorithms to carry out all or a portion of the results of a response likelihood assessment as described herein. Where the computer-readable medium contains a complete program for carrying out the methods described herein, the program includes program instructions for collecting, analyzing and generating output, and generally includes computer readable code devices for interacting with a user as described herein, processing that data in conjunction with analytical information, and generating unique printed or electronic media for that user.

Where the storage medium provides a program which provides for implementation of a portion of the methods described herein (e.g., the user-side aspect of the methods (e.g., data input, report receipt capabilities, etc.)), the program provides for transmission of data input by the user (e.g., via the internet, via an intranet, etc.) to a computing environment at a remote site. Processing or completion of processing of the data can be carried out at the remote site to generate a report. After review of the report, and completion of any needed manual intervention, to provide a complete report, the complete report can be then transmitted back to the user as an electronic document or printed document (e.g., fax or mailed paper report). The storage medium containing a program as described herein can be packaged with instructions (e.g., for program installation, use, etc.) recorded on a suitable substrate or a web address where such instructions may be obtained. The computer-readable storage medium can also be provided in combination with one or more reagents for carrying out response likelihood assessment (e.g., primers, probes, arrays, or other such kit components).

### Output

In some embodiments, once a score for a particular sample (patient) is determined, an indication of that score can be displayed and/or conveyed to a clinician or other caregiver. For example, the results of the test are provided to a user (such as a clinician or other health care worker, laboratory personnel, or patient) in a perceivable output that provides information about the results of the test. In some examples, the output is a paper output (for example, a written or printed output), a display on a screen, a graphical output (for example, a graph, chart, or other diagram), or an audible output.

For example, the output can be textual (optionally, with a corresponding) score. For example, textual outputs may be “consistent with squamous NSCLC” or the like, or “consistent with non-squamous NSCLC” or the like, or “indeterminant” (e.g., not consistent with either squamous or non-squamous NSCLC) or the like. Such textual output can be used, for example, to provide a diagnosis of squamous or nonsquamous NSCLC, or can simply be used to assist a clinician in distinguishing a squamous NSCLC from nonsquamous NSCLC subtypes.

In other examples, the output is a numerical value, such as an amount of gene or protein expression (such as those in any of Tables 2-6) in the sample or a relative amount of gene or protein expression (such as those in any of Tables 2-6) in the sample as compared to a control. In additional examples, the output is a graphical representation, for example, a graph that indicates the value (such as amount or relative amount) of gene or protein expression (such as those in any of Tables 2-6) in the sample from the subject on a standard curve. In a particular example, the output (such as a graphical output) shows or provides a cut-off value or level that indicates the presence of squamous NSCLC or nonsquamous NSCLC. In some examples, the output is communicated to the user, for example by providing an output via physical, audible, or electronic means (for example by mail, telephone, facsimile transmission, email, or communication to an electronic medical record).

The output can provide quantitative information (for example, an amount of gene or protein expression (such as those in any of Tables 2-6), for example relative to a control sample or value, or amount of gene or protein expression (such as those in any of Tables 2-6) or can provide qualitative information (for example, diagnosis of squamous NSCLC or nonsquamous NSCLC). In additional examples, the output can provide qualitative information regarding the relative amount of gene or protein expression (such as those in any of Tables 2-6) in the sample, such as identifying presence of an increase in gene or protein expression (such as those in any of Tables 2-6) relative to a control, a decrease in gene or protein expression (such as those in any of Tables 2-6) relative to a control, or no change in gene or protein expression (such as those in any of Tables 2-6) relative to a control.

In some examples, the output is accompanied by guidelines for interpreting the data, for example, numerical or other limits that indicate the presence or absence of primary melanoma. The guidelines need not specify whether squamous or nonsquamous NSCLC, is present or absent, although it may include such a diagnosis. The indicia in the output can, for example, include normal or abnormal ranges or a cutoff, which the recipient of the output may then use to interpret the results, for example, to arrive at a diagnosis or treatment plan. In other examples, the output can provide a recommended therapeutic regimen. In some examples, the test may include determination of other clinical information (such as determining the amount of one or more additional melanoma biomarkers in the sample).

### Exemplary System for Automating Nuclease Protection Embodiments

In some embodiments, an automated system will provide users of disclosed classifiers with one exemplary reliable platform for reproducibly performing qNPA assays and implementing disclosed classifiers using that representative technology.

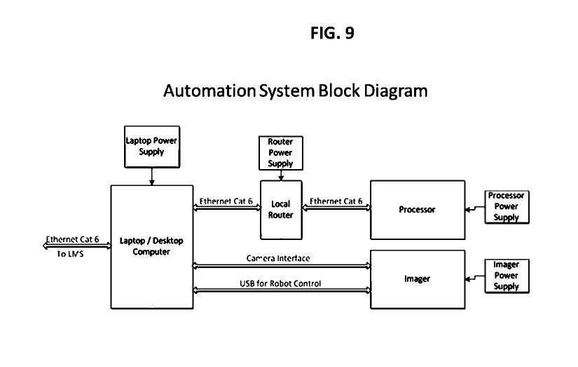

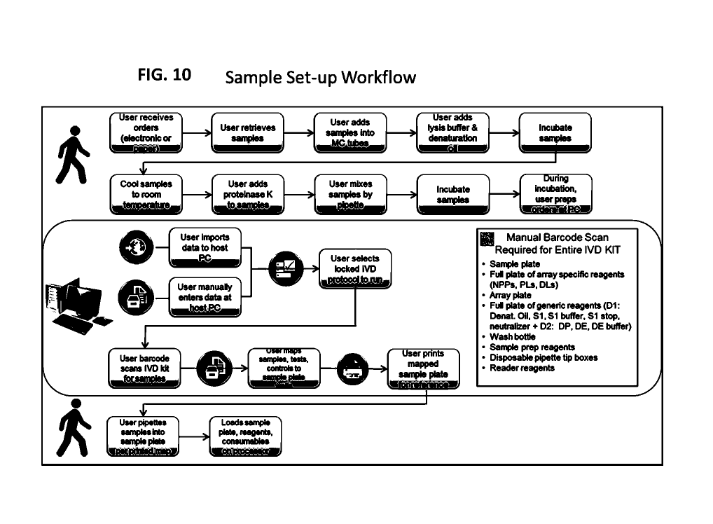

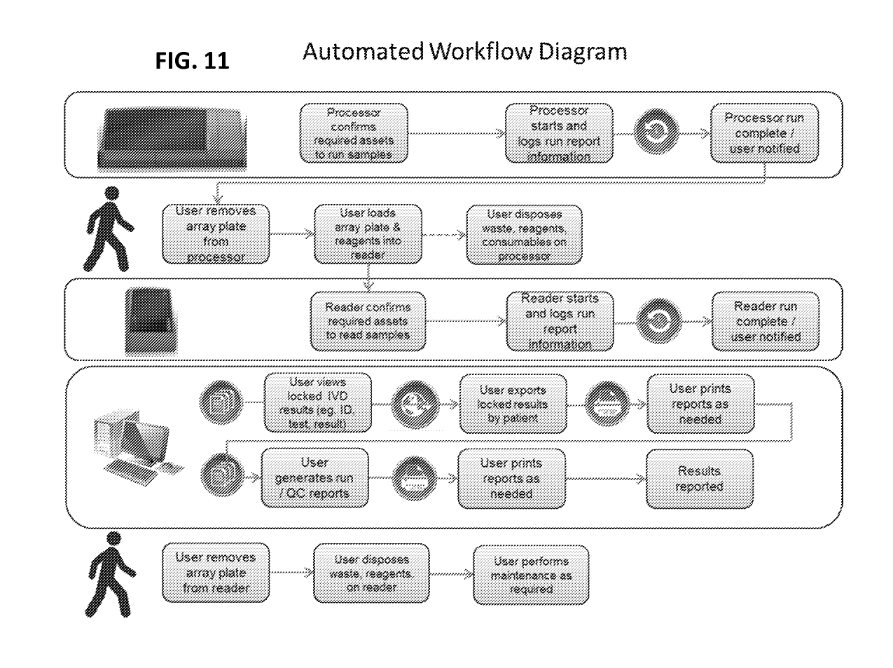

An embodiment of the instrumentation comprises an automated liquid handling unit (Processor), an automated liquid handling and imaging unit (Imager), and a personal computing (PC) workstation (see FIG. 9). As shown in one representative workflow diagramed in FIG. 10, users prepare samples and interact with the system by loading onto it a sample plate pre-loaded with samples to be tested (e.g., human patient samples), reagent trays, assay consumables, and a detection plate (e.g., ArrayPlate). The PC is used to select the appropriate assay protocol for each sample plate loaded in the Processor. FIG. 11 shows a complete step-by-step automation workflow embodiment.

Processor

A sample plate containing samples, for which detection of gene expression products (e.g., RNA) is desired, is placed into the Processor together with the consumables necessary for the desired assay. An instruction set with the necessary commands required for the assay will be sent to the Processor from the PC based on the assay selected by the user. The instruction set will perform the necessary steps to complete the assay. When processing of the detection plate (e.g., ArrayPlate) is complete, the detection plate (e.g., ArrayPlate) will be taken out of the Processor by the user and placed into the Imager for imaging and quantitation.

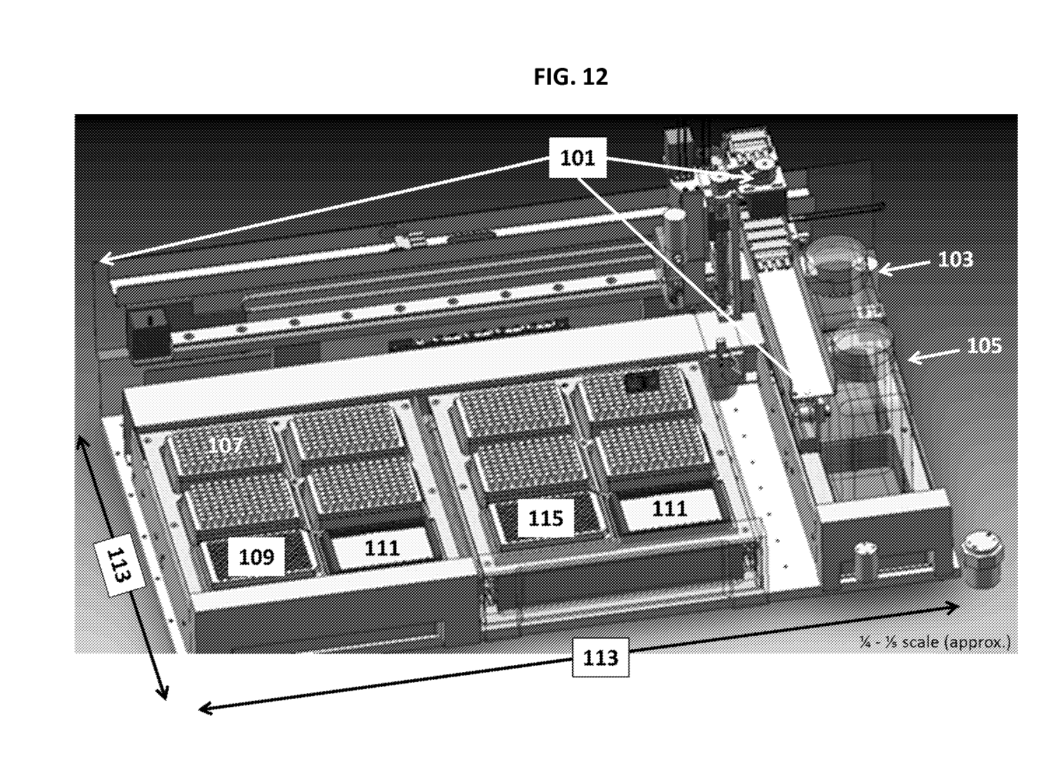

FIG. 12 is a schematic of an exemplary Processor, which comprises a foundation base 113 upon which is stably mounted a positioning robot 101 (e.g., as described in U.S. Pat. Pub. No. 20120152050 and incorporated herein in its entirety). The positioning robot is capable of moving a multi-channel (e.g., 8-channel) pipetting manifold in the x, y and z axes. The foundation base also stably supports (i) at least one e.g., one or two) sample-plate platform 109 of suitable size and shape to receive and support a sample plate (e.g., 6, 24, 96, 384-well microtiter plate); (ii) at least one (e.g., one or two) detection-plate platform 115 of suitable size and shape to receive and support a detection plate (e.g., 6, 24, 96, 384-well microtiter plate (such as a 96-well ArrayPlate)); (iii) one or more (e.g., 2, 3, 4, 5, 6, 7, or up to 8) containers of pipette tips 107; (iv) at least one (e.g., one or two) assay-reagent platforms 111 to receive and support reagent trays from which assay reagents may be collected into pipette tips by the pipetting manifold; (v) a bulk liquid (e.g., wash buffer) reservoir 103, and (vi) a liquid waste reservoir 105.

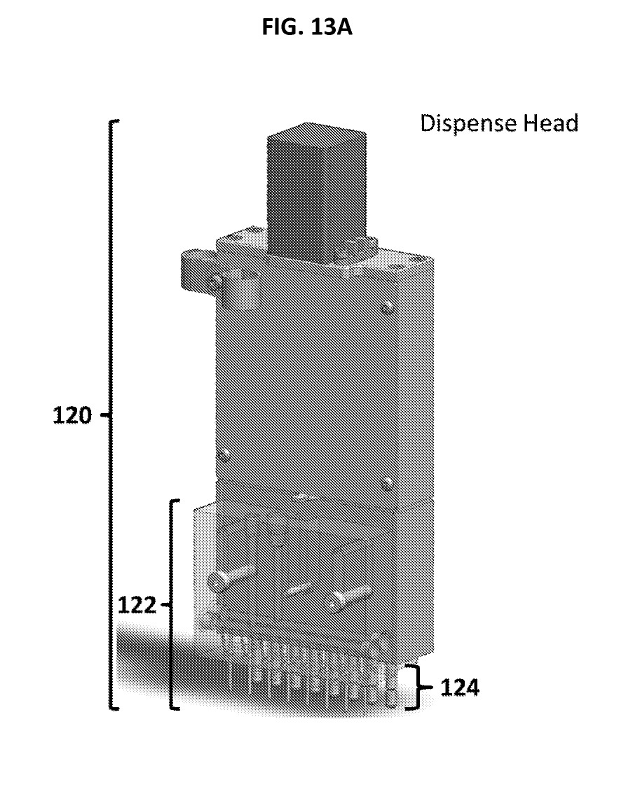

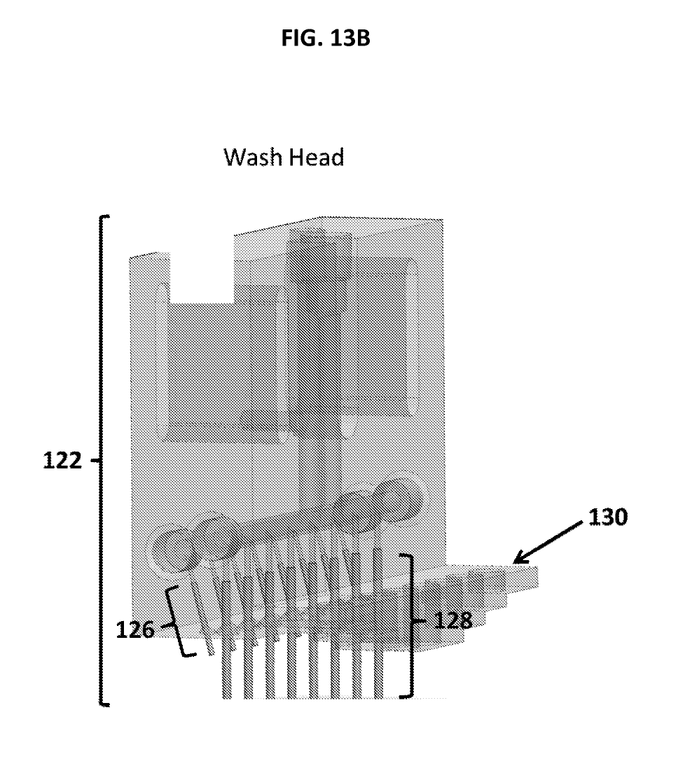

A representative pipetting manifold 120 is shown in greater detail in FIG. 13. It comprises multiple pipetters 124 and a wash head 122. Each of the multiple pipetters (e.g., up to 8 pipetters) is capable of receiving a single pipette tip, collecting in the pipette tip a specified amount of reagent (e.g., assay reagent), and dispensing from such pipette tip such reagent to a specified well of a sample or detection plate. Pipettors 126 are aligned and stabilized by a molded part 130 shown in FIG. 13B. The pipette manifold 120 is designed with a number of pipetters to match the arrangement of pipette tips in a pipette tip container; thus, for example, a pipette manifold suitable for use with pipette tip containers having 8 rows of 12 pipette tips will have 8 or 12 pipetters, as is appropriate for the system-level operation of the Processor. The pipetting manifold, optionally, has a mechanical mechanism to remove (e.g., eject) pipette tips from the pipetters. As shown in detail in FIG. 13B, the wash head 122 comprises dispensing needles (or tubes) 126 and aspirate needles (or tubes) 128. In an one embodiment, each dispensing needle 126 is mounted at an angle (e.g., 10 to 20 degrees, such as 15 degrees) to a corresponding aspirate needle 128, such that fluids (e.g., wash buffer) dispensed from each dispensing needle strikes the corresponding aspirate needle so that the dispensed fluid (by surface tension) flows down the aspirate needle to the intended destination (e.g., sample- or detection-plate well). The wash head 122 is in fluid contact, e.g., through a system of tubing, with the wash buffer reservoir 103 and the waste dispenser 105.

Sample-plate 109 and/or detection-plate platform(s) 115, optionally, may be heated or cooled and/or be capable of mixing reactions in the sample or detection plates, as applicable. Heating and/or cooling are/is controllable within desirable temperature ranges (such as, from 20-95° C.). Mixing of reactions may be accomplished using any mechanical, electrical, magnetic or other means then-available, including, for example, magnetic stirring of individual well contents, and/or rocking or vibrating of the sample plate.

Pipette tip containers (e.g., boxes) 107 comprising a plurality of individual pipette tips mounted in the container in a manner that aligns, in whole or in part, with the pipetters in the pipetting manifold. In a preferred embodiment, a pipette tip box holds 96 individual pipette tips in an 8×12 configuration, and each pipette tip can dispense up to 165 ul (e.g., 2-300 ul, 2-100 ul, 20-100 ul, or less than 100 ul).

Reagent trays also are designed to hold liquid reagents in a manner that aligns, in whole or in part, with the pipetters in the pipetting manifold. Reagent trays are situated on the assay-reagent platform(s) 111. In a preferred embodiment, each reagent is present in a trough that is of sufficient length and depth to physically accommodate the distal ends of all pipette tips fit to the pipetters to a depth sufficient for the specified amount of liquid to be collected from the trough into the pipette tips.

In operation, the robot will position the pipetters 124 over the pipette tip container 107 and lower the pipetters sufficient distance and with sufficient force to pick up by compression fit the pipette tips from the specified pipette container. The robot, then, will raise the pipette tips to a vertical point where such tips can move free of obstruction. As specified by system software, the robot will position the pipette tips over the appropriate trough in the reagent tray 111 and lower the pipette tips into the liquid reagent to a sufficient depth to permit the pipetting manifold 120 to collect in the pipette tips 124 the called-for amount of reagent (e.g., using positive displacement with a piston pin). Reagents may include any liquid reagent useful at any and every point in the desired assay (e.g., S1 nuclease buffer, oil to prevent evaporation or the like). The reagent-containing pipettes will be raised, retaining the reagent, and moved (free of obstruction) to a desired position, such as above the appropriate wells in the sample or detection plate; in which event, the robot will lower the reagent-containing pipette tips to an appropriate depth above the bottom of the corresponding sample- or detection-plate wells and dispense the reagent into the appropriate wells (e.g., by positive displacement). Similarly, when the system calls for one or more wash steps, the robot will position the wash head in and at an appropriate height above the bottom of the receiving well (e.g., to avoid constriction, splashing or overspray) and dispense liquid (e.g., wash buffer) received (via tubing or other plumbing) from the wash buffer reservior through the dispensing needles 126 (optionally, via surface tension down the aspirate needles 128) to the receiving well. Liquid marked by system software for disposal (e.g., spent assay reagents or wash buffer) is managed by the robot lowering the aspirate needles 128 into identified reagent-containing wells, wherein the aspirate needles collecting liquid from the wells for transmission (via tubing and suction or otherwise) to the waste container 105.

Imager

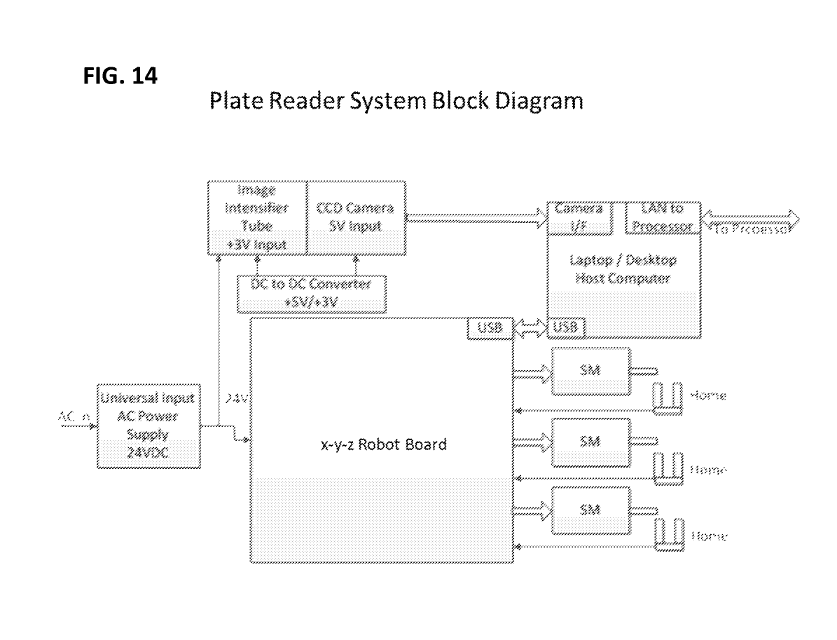

Upon completion of the Processor instruction set, a user can remove the detection plate (e.g., ArrayPlate) from the Processor and place such detection plate into the Imager for imaging and quantitation. A block diagram for an exemplary Imager is shown in FIG. 14. The automation workflow relating to the Imager is shown in FIG. 11 (see, rows 2, 3 and 5).

Imager processing can include mechanical elements (e.g, x-y-z robot with pipetting capabilities) for the adding of detection reagents and imaging oil to the wells of the detection plate (e.g., ArrayPlate) and imaging elements (e.g., image intensifier tube and CCD camera) for capturing the light output of each of the individual array elements in each micro titer plate well and converting to relative light units (RLUs). The processing can begin by the automated mixing of luminescent substrate A and B reagents; once mixed, the luminescent substrate can be added to the applicable detection plate wells and each such well layered with imaging oil to prevent evaporation of detection reagents. The Imager software can schedule the timing of reagent application and image capture from each well of the detection plate (e.g., ArrayPlate) to ensure a consistent application and image.

Software

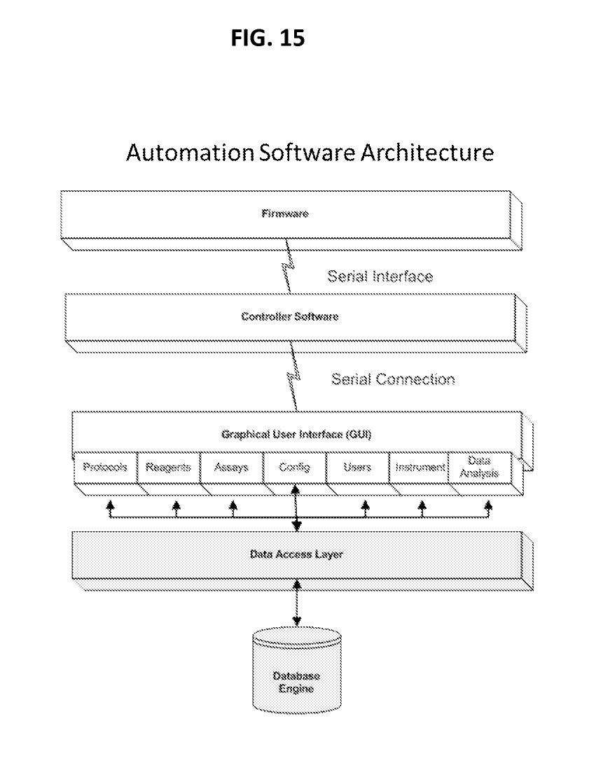

The exemplary automation system software (see, FIG. 15) can include an operating system-based (e.g., Microsoft Windows) Host-PC software (Host), Controller software (ICP), embedded software (Firmware), and assay procedures used to control the system processing module(s). The Host and Controller components of the Processer communicate via an Ethernet interface through a Cat 6 cable using a standard communications protocol. The standard protocol provides an interface for command and control, and monitoring of the processing system(s). The Controller and Firmware components of the Processor communicate via a USB interface through a USB cable using a standard USB communications protocol. The standard protocol provides an interface for command and control, and monitoring of the embedded system.

The Host and Controller components of the Plate Reader communicate via a USB interface through a USB cable using a standard USB communications protocol. The standard protocol provides an interface for command and control, and monitoring of the imaging system.

The Host software provides the graphical user interface (GUI) for the automation processing and imaging systems and provides users with the ability to configure, administer, command, control and monitor up to eight (8) different processing instruments and a single plate reader system connected to a single host computer.

Representative Host software architecture can include or consist of a multiple tiers that will provide a modular application.

Presentation Layer: The presentation layer represents the interface between the user and the rest of the application. The Presentation layer displays data and accepts user input via keystrokes and mouse gestures and manages application-specific navigation issues. The Presentation layer can utilize the .NET framework and the C# programming language to provide the graphical user interface for the exemplary automation platform systems and provides users with the ability to configure, administer, command, control and monitor all connected automation system units.

Business Logic Layer: The business logic layer logic can be concerned with the retrieval, processing, transformation, and management of application data, application of business rules and policies, and ensuring data consistency and validity. The business logic layer can utilize web services and libraries to retrieve the data, process the data, and transport the data to the proper requestor of the data. Each major functional activity can become a separate module, including the processing engine module for each supported instrument (Processor and Plate Reader).

The benefit for separating the processing engine module into discrete libraries for a processing and imaging instrument is the ability to change the process for one supported instrument without impacting the process for the other instrument. This architectural approach preserves the integrity of the software validation for the unaffected processing engine modules supporting any and all assays for which the system is programmed.

This architectural approach also allows functional changes to be made to other functional modules without impacting the processing engine modules associated with any other automated assays for which the system is designed.

Data Access Layer: The data access layer encapsulates the data access logic and data access technologies used. It also separates the data access logic from business logic. The data access layer can provide a generic interface for database operations. The Data Access layer manages persistent storage of data to a database. The Data Access layer provides data to the consumers of the data, which will usually be the business layer and could be a service or even a business process.

Controller Layer: The controller layer will utilize a programming language (e.g., Python) to provide communications directly to the Firmware and Host PC while residing on the instrument. The controller layer is a PC-based application and is responsible for accepting an assay-specific instruction set from the host system and executing the instruction set on the instrument. The controller layer can have an interface to the firmware and manage the interaction with the devices within the instrument. The controller layer can monitor the instrument sensors, interlocks, and devices for expected behaviors and report errors back to the host system when an error occurs.

Firmware Layer: The firmware layer can utilize a programming language (e.g., C programming language) to provide communications to devices and components on the instrument. The firmware is loaded into a microprocessor chip that resides on a printed circuit board (PCB) within the instrument. The firmware layer accepts an instruction from the controller and executing the instruction on the instrument.

### Clinical Use of Gene Sets and Classifier Outputs

The disclosed gene sets or classifiers may result in a sample being characterized (e.g., diagnosed) as not NSCLC, squamous NSCLC, nonsquamous NSCLC (e.g., adenocarcinoma or large cell carcinoma), colon-originating lung cancer, in the group of small cell lung cancer and pulmonary carcinoids), indeterminate or suspicious (suggestive of a cancer, disease, or condition), or non-diagnostic (e.g., providing inadequate information concerning the presence or absence of a cancer, disease, or condition). Each of these (and other possible) results is useful to the trained clinical professional. Some representative clinical uses are described in more detail below.

### Diagnosis Indications

A diagnosis informs a subject (e.g., patient) what disease or condition s/he has or may have. As more particularly described throughout this disclosure, any result of any disclosed method that identifies a lung malignancy (or subtype thereof) can be provided, e.g., to a subject or health professional, as a diagnosis.

### Prognostic Indications

Prognosis is the likely health outcome for a subject whose sample received a particular test result (e.g., squamous cell NSCLC versus nonsquamous NSCLC). A poor prognosis means the long-term outlook for the subject is not good, e.g., the 1-, 2-, 3- or 5-year survival is 50% or less (e.g., 40%, 30%, 25%, 20%, 15%, 10%, 5%, 2% or 1% or less). On the other hand, a good prognosis means the long-term outlook for the subject is fair to good, e.g., the 1-, 2-, 3- or 5-year survival is greater than 30%, 40%, 50%, 60%, 70%, 75%, 80% or 90%.

Squamous cell NSCLC has been shown to have a poorer prognosis than many types of non-squamous NSCLC. Accordingly, a finding of squamous cell NSCLC by any of the disclosed methods can be used to predict a comparatively poor prognosis for a subject from whom the test sample is taken. Conversely, a finding of nonsquamous NSCLC (e.g., adenocarcinoma NSCLC) by any of the disclosed methods can be used to predict a comparatively good prognosis for the corresponding subject.

### Therapeutic (Predictive) Indications

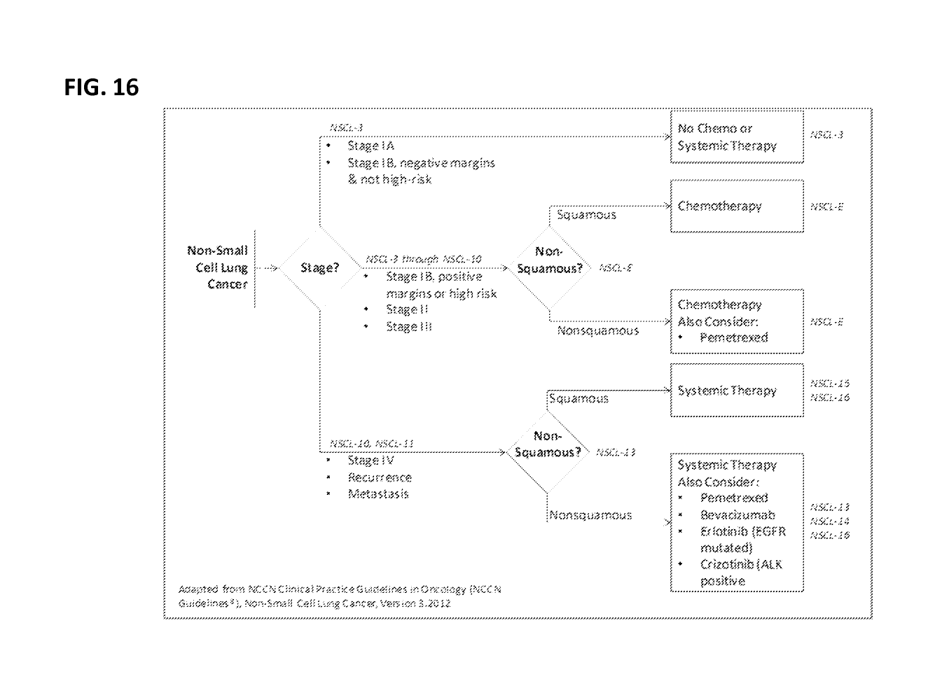

The disclosed methods can further include selecting (or not selecting) subjects for treatment for squamous cell NSCLC or nonsquamous cell NSCLC, if their corresponding sample is so subtyped. FIG. 16 shows various treatment options presently known for NSCLC patients and the different regimes for such patients depending upon the cancer stage and whether their NSCLC is the squamous or nonsquamous subtype. Each of the series of steps and corresponding treatments shown in FIG. 16 may be included in specific method embodiments. Thus, in one example, if the sample is determined to be non-squamous NSCLC, the subject from whom the sample was obtained is treated with Pemetrexed. In another example, if the sample is determined to be squamous NSCLC, the subject from whom the sample was obtained is not treated with Pemetrexed due to the toxicity of the drug in this patient population.

In some embodiments, disclosed methods also include one or more of the following depending on the patient's diagnosis: a) prescribing a treatment regimen for the subject if the subject's determined diagnosis is positive for squamous NSCLC (such as treatment with one or more chemotherapeutic agents or systemic therapy; in some cases, further depending upon what is the stage of the patient's NSCLC); b) prescribing a treatment regimen for the subject if the subject's determined diagnosis is positive for nonsquamous NSCLC (Cisplatin/Pemetrexed have superior efficacy and reduced toxicity for nonsquamous NSCLC); or c) not prescribing a treatment regimen for the subject if the subject's determined diagnosis is squamous cell NSCLC (for example, EGFR mutation and ALK testing are not routinely recommended for squamous NSCLC, or Bevacizumab plus chemotherapy is not recommended for squamous NSCLC.

### Arrays

Disclosed herein are arrays that can be used to detect gene expression (such as expression of two or more of the biomarkers in any of Tables 2-6), for example for use in subtyping a lung sample as squamous NSCLC or nonsquamous NSCLC (or as not a NSCLC) as discussed above. In some embodiments, the disclosed arrays can also be used to detect expression of one or more normalization biomarkers (e.g., those in Table 7). In particular examples, the array surface includes a plate, bead, or flow cell.

In some embodiments an array can include a solid surface including specifically discrete regions or addressable locations, each region having at least one immobilized oligonucleotide capable of directly hybridizing to at least two different biomarkers in any of Tables 2-6 (such as Tables 2-4), and in some examples to a normalization gene shown in Table 7. The oligonucleotide probes are identifiable by position on the array. In another example, an array can include specifically discrete regions, each region having at least one or at least two immobilized capture probes. The immobilized capture probes are capable of directly or indirectly specifically hybridizing with at least two different biomarkers in any of Tables 2-6 (such as Tables 2-4), and in some examples to at least one normalization gene shown in Table 7. The capture probes are identifiable by position on the array. The probes on the array can be attached to the surface in an addressable manner. For example, each addressable location can be a separately identifiable bead or a channel in a flow cell.

In one example an array includes a solid surface including specifically discrete regions or addressable locations, each region having at least one immobilized oligonucleotide capable of directly hybridizing to at least 2, at least 3, at least 5, at least 10, at least 20 or all 28 biomarkers in Table 3, and in some examples to at least one normalization gene shown in Table 7 (such as 1, 2, 3, 4, 5, 6, 7, 8, 9, 10 or all of the normalization gene shown in Table 7). For example, the array can include specifically discrete regions, each region having at least one or at least two immobilized capture probes capable of directly or indirectly specifically hybridizing with at least 2, at least 3, at least 5, at least 10, at least 20 or all 28 biomarkers in Table 3, and in some examples to at least one normalization gene shown in Table 7 (such as 1, 2, 3, 4, 5, 6, 7, 8, 9, 10 or all of the normalization genes shown in Table 7).

In one example an array includes a solid surface including specifically discrete regions or addressable locations, each region having at least one immobilized oligonucleotide capable of directly hybridizing to 2, 3, 4, 5, 6, 5, or all 8 biomarkers in Table 4, and in some examples to at least one normalization gene shown in Table 7 (such as 1, 2, 3, 4, 5, 6, 7, 8, 9, 10 or all of the normalization gene shown in Table 7). For example, the array can include specifically discrete regions, each region having at least one or at least two immobilized capture probes capable of directly or indirectly specifically hybridizing with 2, 3, 4, 5, 6, 5, or all 8 biomarkers in Table 4, and in some examples to at least one normalization gene shown in Table 7 (such as 1, 2, 3, 4, 5, 6, 7, 8, 9, 10 or all of the normalization genes shown in Table 7).

In one example an array includes a solid surface including specifically discrete regions or addressable locations, each region having at least one immobilized oligonucleotide capable of directly hybridizing to at least 2, at least 3, at least 5, at least 10, at least 15, or all 17 biomarkers in Table 5, and in some examples to at least one normalization gene shown in Table 7 (such as 1, 2, 3, 4, 5, 6, 7, 8, 9, 10 or all of the normalization gene shown in Table 7). For example, the array can include specifically discrete regions, each region having at least one or at least two immobilized capture probes capable of directly or indirectly specifically hybridizing with at least 2, at least 3, at least 5, at least 10, at least 15, or all 17 biomarkers in Table 5, and in some examples to at least one normalization gene shown in Table 7 (such as 1, 2, 3, 4, 5, 6, 7, 8, 9, 10 or all of the normalization genes shown in Table 7).

In one example an array includes a solid surface including specifically discrete regions or addressable locations, each region having at least one immobilized oligonucleotide capable of directly hybridizing to 1, 2, 3, 4, 5, or all 6 biomarkers in Table 6, and in some examples to at least one normalization gene shown in Table 7 (such as 1, 2, 3, 4, 5, 6, 7, 8, 9, 10 or all of the normalization genes shown in Table 7). For example, the array can include specifically discrete regions, each region having at least one or at least two immobilized capture probes capable of directly or indirectly specifically hybridizing with 1, 2, 3, 4, 5, or all 6 biomarkers in Table 6, and in some examples to at least one normalization gene shown in Table 7 (such as 1, 2, 3, 4, 5, 6, 7, 8, 9, 10 or all of the normalization gene shown in Table 7).

In one example an array includes a solid surface including specifically discrete regions or addressable locations, each region having at least one immobilized oligonucleotide capable of directly hybridizing to all 28 biomarkers in Table 3, the first 6 normalization biomarkers in Table 7, biomarkers SFTPB, CLRN3, CDH17, LGALS4, and CXCL17 in Table 5, and the 6 biomarkers in Table 6. For example, the array can include specifically discrete regions, each region having at least one or at least two immobilized capture probes capable of directly or indirectly specifically hybridizing with all 28 biomarkers in Table 3, the first 6 normalization biomarkers in Table 7, biomarkers SFTPB, CLRN3, CDH17, LGALS4, and CXCL17 in Table 5, and the 6 biomarkers in Table 6.

For example, the array can include at least three addressable locations, each location having immobilized capture probes with the same specificity, and each location having capture probes having a specificity that differs from capture probes at each other location. The capture probes at two of the at least three locations are capable of directly or indirectly specifically hybridizing a biomarker listed in any of Tables 2-6, and the capture probes at one of the at least three locations is capable of directly or indirectly specifically hybridizing a normalization biomarker listed in Table 7. In addition, the specificity of each capture probe is identifiable by the addressable location the array. In some examples the array further includes at least two discrete regions (such wells on a multi-well surface, or channels in a flow cell), each region having the at least three addressable locations. In some example, such an array includes immobilized capture probes capable of directly or indirectly specifically hybridizing with all 28 biomarkers listed in Table 3 and the first 6 normalization biomarkers in Table 7, and optionally biomarkers SFTPB, CLRN3, CDH17, LGALS4, and CXCL17 in Table 5, and all 6 biomarkers in Table 6. In some examples, the capture probe(s) indirectly hybridize with the at least two biomarkers listed in any of Tables 2-6 and the at least one normalization biomarker in Table 7 through a nucleic acid programming linker, wherein the programming linker is a hetro-bifunctional linker which has a first portion complementary to the capture probe(s) and a second portion complementary to a nuclease protection probe (NPP), wherein the NPP is complementary to one of the at least two biomarkers listed in any of Tables 2-6 or the at least one normalization biomarker in Table 7 Thus, in some examples the array also includes the nucleic acid programming linkers.

In some embodiments the array includes oligonucleotides that include or consist essentially of oligonucleotides that are complementary to at least 2 at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, 28 of the biomarkers in Table 3 (such as 2, 3, 4, 5, 6, 7, 8, 9, 10, 11, 12, 13, 14, 15, 16, 17, 18, 19, 20, 21, 22, 23, 24, 25, 26, 27, or 28 of the biomarkers in Table 3

In some examples, the array further includes oligonucleotides that are complementary to normalization biomarkers, such as at least 1, at least 2 at least 3, at least 4, at least 5, at least 6, at least 7, at least 8, at least 9, at least 10 or all of the biomarkers in Table 7 (such as 1, 2, 3, 4, 5, or 6 of the normalization biomarkers in Table 7). In some examples, the array further includes oligonucleotides that are complementary to biomarkers SFTPB, CLRN3, CDH17, LGALS4, and CXCL17 in Table 5, and/or all 6 biomarkers in Table 6. In some examples, the array further includes one or more control oligonucleotides (such as 1, 2, 3, 4, 5, 6, 7, 8, 9, 10, or more control oligonucleotides), for example, one or more positive and/or negative controls. In some examples, the control oligonucleotides are complementary to one or more of DEAD box polypeptide 5 (DDX5), glyceraldehyde-3-phosphate dehydrogenase (GAPDH), fibrillin 1 (FBN1), or Arabidopsis thaliana AP2-like ethylene-responsive transcription factor (ANT).

In some embodiments, the array can include a surface having spatially discrete regions (such as wells on a multi-well surface, or channels in a flow cell), each region including an anchor stably (e.g., covalently) attached to the surface and nucleic acid programming linker, wherein the programming linker is a hetro-bifunctional linker which has a first portion complementary to the capture probe(s) and a second portion complementary to a nuclease protection probe (NPP), wherein the NPP is complementary to a target nucleic acid (such as those in any of Tables 2-6). In some embodiments the array includes or consists essentially of bifunctional linkers in which the first portion is complementary to an anchor and the second portion is complementary to an NPP, wherein the NPP is complementary to one of the at least 2 at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, or all 28 of the biomarkers in Table 3 (such as 2, 3, 4, 5, 6, 7, 8, 9, 10, 11, 12, 13, 14, 15, 16, 17, 18, 19, 20, 21, 22, 23, 24, 25, 26, 27, or 28 of the biomarkers in Table 3). In some examples, the array further includes bifunctional linkers in which the first portion is complementary to an anchor and the second portion is complementary to an NPP complementary to a normalization biomarker, such as at least 1, at least 2 at least 3, at least 4, at least 5, or the first 6 or all of the biomarkers in Table 7 (such as 1, 2, 3, 4, 5, 6, 7, or 8 of the biomarkers in Table 7). In some examples, the array further includes bifunctional linkers in which the first portion is complementary to an anchor and the second portion is complementary to an NPP complementary to a another biomarker, such as at least 2, at least 3, at least 5, at least 10, at least 15, or all 17 biomarkers in Table 5. In some examples, the array further includes bifunctional linkers in which the first portion is complementary to an anchor and the second portion is complementary to an NPP complementary to a another biomarker, such as 1, 2, 3, 4, 5, or all 6 biomarkers in Table 6. Such arrays have attached thereto the anchor hybridized to at least a segment of the bifunctional linker that is not complementary to the NPP. In another example, the array further includes bifunctional linkers in which the second portion of the bifunctional linker is complementary to an NPP complementary to a control gene (such as DDX5, GAPDH, FBN1, or ANT). Such arrays can further include (1) the anchor probe hybridized to the first portion of the programming linker, (2) NPPs hybridized to the second portion of the programming linker, (3) bifunctional detection linkers having a first portion hybridized to the NPPs and a second portion hybridized to a detection probe, (4) a detection probe; (5) a label (such as avidin HRP), or combinations thereof.

In some examples, a collection of up to 47 different capture (i.e., anchor) oligonucleotides can be spotted onto the surface at spatially distinct locations and stably associated with (e.g., covalently attached to) the derivatized surface (e.g., to detect the 47 markers in Table 8). For any particular assay, a given set of capture probes can be used to program the surface of each well to be specific for as many as 47 different targets or assay types of interest, and different test samples can be applied to each of the 96 wells in each plate. The same set of capture probes can be used multiple times to re-program the surface of the wells for other targets and assays of interest.

### Array Substrates

The solid support of the array can be formed from an organic polymer. Suitable materials for the solid support include, but are not limited to: polypropylene, polyethylene, polybutylene, polyisobutylene, polybutadiene, polyisoprene, polyvinylpyrrolidine, polytetrafluroethylene, polyvinylidene difluroide, polyfluoroethylene-propylene, polyethylenevinyl alcohol, polymethylpentene, polycholorotrifluoroethylene, polysulfornes, hydroxylated biaxially oriented polypropylene, aminated biaxially oriented polypropylene, thiolated biaxially oriented polypropylene, ethyleneacrylic acid, thylene methacrylic acid, and blends of copolymers thereof (see U.S. Pat. No. 5,985,567). Other examples of suitable substrates for the arrays disclosed herein include glass (such as functionalized glass), Si, Ge, GaAs, GaP, SiO₂, SiN₄, modified silicon nitrocellulose, polystyrene, polycarbonate, nylon, fiber, or combinations thereof. Array substrates can be stiff and relatively inflexible (for example glass or a supported membrane) or flexible (such as a polymer membrane).

In general, suitable characteristics of the material that can be used to form the solid support surface include: being amenable to surface activation such that upon activation, the surface of the support is capable of stably (e.g., covalently, electrostatically, reversibly, irreversibly, or permanently) attaching a biomolecule such as an oligonucleotide thereto; amenability to “in situ” synthesis of biomolecules; being chemically inert such that at the areas on the support not occupied by the oligonucleotides or proteins (such as antibodies) are not amenable to non-specific binding, or when non-specific binding occurs, such materials can be readily removed from the surface without removing the oligonucleotides or proteins (such as antibodies).

In another example, a surface activated organic polymer is used as the solid support surface. One example of a surface activated organic polymer is a polypropylene material aminated via radio frequency plasma discharge. Other reactive groups can also be used, such as carboxylated, hydroxylated, thiolated, or active ester groups.

### Array Formats

Within an array, each arrayed sample is addressable, in that its location can be reliably and consistently determined within at least two dimensions of the array. The feature application location on an array can assume different shapes. For example, the array can be regular (such as arranged in uniform rows and columns) or irregular. Thus, in ordered arrays the location of each sample is assigned to the sample at the time when it is applied to the array, and a key may be provided in order to correlate each location with the appropriate target or feature position. Often, ordered arrays are arranged in a symmetrical grid pattern, but samples could be arranged in other patterns (such as in radially distributed lines, spiral lines, or ordered clusters). Addressable arrays usually are computer readable, in that a computer can be programmed to correlate a particular address on the array with information about the sample at that position (such as hybridization or binding data, including for instance signal intensity). In some examples of computer readable formats, the individual features in the array are arranged regularly, for instance in a Cartesian grid pattern, which can be correlated to address information by a computer.

One example includes a linear array of oligonucleotide bands, generally referred to in the art as a dipstick. Another suitable format includes a two-dimensional pattern of discrete cells (such as 4096 squares in a 64 by 64 array). In one example, the array includes up to 47 (e.g., 5, between 5 and 16, between 5 and 47, 16, between 16 and 47) addressable locations per reaction chamber; thus, in a 96-well array, there may be 96×5, 96×16, 96×47 addressable locations with the addressable locations within each reaction chamber (e.g., well) being the same or different (e.g., using programmable array technologies); provided, however, it is understood in that art that universally programmable arrays may be flexibly programmed to capture any number of analytes up to the number of addressable locations that can physically be printed on the array surface of interest. Other embodiments include arrays comprising physically separate surfaces combined together into a set of surfaces that when combined create an addressable array; for example, a set of individually identifiable (e.g., addressable) beads, each programmed or printed to capture a specific analyte. As is appreciated by those skilled in the art, other array formats including, but not limited to slot (rectangular) and circular arrays are equally suitable for use (see U.S. Pat. No. 5,981,185). In some examples, the array is a multi-well plate (such as a 96-well plate). In one example, the array is formed on a polymer medium, which is a thread, membrane or film. An example of an organic polymer medium is a polypropylene sheet having a thickness on the order of about 1 mil. (0.001 inch) to about 20 mil., although the thickness of the film is not critical and can be varied over a fairly broad range. The array can include biaxially oriented polypropylene (BOPP) films, which in addition to their durability, exhibit low background fluorescence.

The array formats of the present disclosure can be included in a variety of different types of formats. A “format” includes any format to which the solid support can be affixed, such as microtiter plates (e.g., multi-well plates), test tubes, inorganic sheets, dipsticks, beads, and the like. For example, when the solid support is a polypropylene thread, one or more polypropylene threads can be affixed to a plastic dipstick-type device; polypropylene membranes can be affixed to glass slides. The particular format is, in and of itself, unimportant. All that is necessary is that the solid support can be affixed thereto without affecting the functional behavior of the solid support or any biopolymer absorbed thereon, and that the format (such as the dipstick or slide) is stable to any materials into which the device is introduced (such as clinical samples and hybridization solutions).

The arrays of the present disclosure can be prepared by a variety of approaches. In one example, oligonucleotide sequences are synthesized separately and then attached to a solid support (see U.S. Pat. No. 6,013,789). In another example, sequences are synthesized directly onto the support to provide the desired array (see U.S. Pat. No. 5,554,501). Suitable methods for coupling oligonucleotides to a solid support and for directly synthesizing the oligonucleotides onto the support are known to those working in the field; a summary of suitable methods can be found in Matson et al., Anal. Biochem. 217:306-10, 1994. In one example, the oligonucleotides are synthesized onto the support using conventional chemical techniques for preparing oligonucleotides on solid supports (such as PCT applications WO 85/01051 and WO 89/10977, or U.S. Pat. No. 5,554,501).

A suitable array can be produced using automated means to synthesize oligonucleotides in the cells of the array by laying down the precursors for the four bases in a predetermined pattern. Briefly, a multiple-channel automated chemical delivery system is employed to create oligonucleotide probe populations in parallel rows (corresponding in number to the number of channels in the delivery system) across the substrate. Following completion of oligonucleotide synthesis in a first direction, the substrate can then be rotated by 90° to permit synthesis to proceed within a second set of rows that are now perpendicular to the first set. This process creates a multiple-channel array whose intersection generates a plurality of discrete cells.

The oligonucleotides can be bound to the support by either the 3′-end of the oligonucleotide or by the 5′ end of the oligonucleotide. In one example, the oligonucleotides are bound to the solid support by the 3′-end. However, one of skill in the art can determine whether the use of the 3′-end or the 5′-end of the oligonucleotide is suitable for bonding to the solid support. In general, the internal complementarity of an oligonucleotide probe in the region of the 3′-end and the 5′-end determines binding to the support.

### Kits

Also disclosed herein are kits for that can be used to detect expression (such as expression of two or more of the biomarkers in any of Tables 2-6), for example for use in characterizing a sample as a squamous or nonsquamous NSCLC as discussed above. In some embodiments, the disclosed kits can also be used to detect expression of one or more normalization biomarkers (e.g., those in Table 7). In some embodiments, the disclosed kits can be used to detect expression of the 47 markers in Table 8. In particular examples, the kit includes one or more of the arrays provided herein (such as an array that permits detection of the 47 markers in Table 8).

In some examples the kits include probes and/or primers for the detection of nucleic acid or protein expression, such as two or more of the biomarkers in any of Tables 2-6, and in some examples, one or more normalization biomarkers in Table 7. In some examples, the kits include antibodies that specifically bind to biomarkers listed in any of Tables 2-6, and optionally antibodies that specifically bind to one or more normalization biomarkers (e.g., see Table 7). For example, the kits can include one or more nucleic acid probes needed to construct an array for detecting the biomarkers disclosed herein.

In some examples, the kit includes nucleic acid programming linkers. The programming linkers are hetro-bifunctional having a first portion complementary to the capture probe(s) on the array and a second portion complementary to a nuclease protection probe (NPP), wherein the NPP is complementary to one of the at least two biomarkers listed in any of Tables 2-6 or to at least one normalization biomarker in Table 7. In one example, the programming linkers are pre-hybridized to the capture probes, such that they are not covalently attached so that the surface includes the addressable immobilized capture probes and the nucleic acid programming linkers. In such an example, the kit does not have a separate container with programming linkers

In some examples, the kit includes NPPs. The NPPs are complementary to the second portion of the programming linker. Exemplary NPPs are shown in SEQ ID NOS: 1-47.

In some examples, the kit includes bifunctional detection linkers. Such linkers can be labeled with a detection probe and are capable of specifically hybridizing to the NPPs or to the target (such as those in any of Tables 2-6). In some examples, such linkers can be labeled with a detection probe and are capable of specifically hybridizing to at least one normalization maker, such as one or more of those in Table 7).

In some examples, the kit includes an array disclosed herein, and one or more of a container containing a buffer (such as a lysis buffer); a container containing a nuclease specific for single-stranded nucleic acids; a container containing nucleic acid programing linkers; a container containing NPPs; a container containing a plurality of bifunctional detection linkers; a container containing a detection probe (such as one that is triple biotinylated); and a container containing a detection reagent (such as avidin HRP).

In one example, the kit includes a graph or table showing expected values or ranges of values of the biomarkers in any of Tables 2-6 expected in NSCLC squamous and/or nonsquamous subtypes. In some examples, kits further include control samples, such as particular quantities of nucleic acids or proteins for those biomarkers in Table 7.

The kits may further include additional components such as instructional materials and additional reagents, for example detection reagents, such as an enzyme-based detection system (for example, detection reagents including horseradish peroxidase or alkaline phosphatase and appropriate substrate), secondary antibodies (for example antibodies that specifically bind the primary antibodies that specifically bind the proteins in any of Tables 2-6, or antibodies that specifically bind the primary antibodies that specifically bind the normalization proteins in Table 7), or a means for labeling antibodies. The kits may also include additional components to facilitate the particular application for which the kit is designed (for example microtiter plates). In one example, the kit of further includes control nucleic acids. Such kits and appropriate contents are well known to those of ordinary skill in the art. The instructional materials may be written, in an electronic form (such as a computer diskette or compact disk) or may be visual (such as video files).

The following examples are provided to illustrate certain particular features and/or embodiments. These examples should not be construed to limit the disclosure to the particular features or embodiments described.

### EXAMPLES

As discussed throughout this disclosure, there exists a need for tools to subtype lung malignancies, particularly to distinguish between squamous and nonsquamous subtypes of NSCLC and/or to identify samples misdiagnosed as NSCLC. FIG. 2 shows an exemplary process map for making such determinations, and the Examples that follow provide representative detail around steps shown in the map.

### Example 1

### Multiple Data Sets Useful for NSCLC Squamous/Nonsquamous Classifier Development

The number of genes expressed in a particular tissue typically range from about 11,000 to about 15,500 (Ramskold, PLOS Comput. Biol., 5(12):e1000598 (2009)). Most expressed genes are irrelevant to cancer distinction in such tissues. Performing gene selection removes a large number of irrelevant genes, which improves the accuracy of cancer classifiers and improves classifier run time efficiency (Lu and Han, Information Systems, 28:234-268 (2003).

Often, for a variety of reasons, gene selection is made on the basis of a single gene expression data set having a small number of samples. This practice introduces a number potential biases into the data, which may affect the broader utility of any classifier based on such data. One way to improve the robustness of a gene expression classifier is to perform gene selection on number of different data sets, as described in this Example.

As described in the following Examples, more than 1000 samples from a number of different laboratories on a variety of different multiplex platforms using frozen or fixed samples were analyzed in parallel to select gene sets significantly differentially expressed in squamous and nonsquamous (e.g., adenocarcinoma and large cell carcinoma) NSCLC samples. These independent gene sets, then, were cross-validated one against the others to generate a consolidated, highly repeatable gene set useful for developing classifiers for distinguishing squamous NSCLC from other lung malignancies, including nonsquamous NSCLC subtypes (e.g., adenocarcinoma and large cell carcinoma), carcinoids and colon-tumor metastases.

The data sets referred to in these Examples are summarized in the Table 1.

TABLE 1
Datasets.

| Citation | | | |
|---|---|---|---|
| and/or GEO | | | |
| Acc. No.¹ | Platform | Sample Subtype | Purpose(s) |
| Data Set 1 | U133 Plus | Cohort 1-FFPE lung | Deriving gene set |
| as described | 2.0 | samples (134): | significantly |
| herein | followed | Adenocardinoma (70), | differentially |
| | by | squamous cell carcinoma | expressed between |
| | ArrayPlate | (64) | squamous(SQ) and |
| | | Cohort 2-FFPE lung | nonsquamous (nSQ) |
| | | samples (162): | NSCLC samples; and |
| | | Adenocarcinoma (73), | Deriving |
| | | squamous cell carcinoma | normalization genes. |
| | | (64) and large cell | |
| | | carcinoma (25) | |
| Bhattacharjee | U95A, v2 | Snap-frozen lung samples | Deriving gene set |
| et al., PNAS, | | (203): Adenocarcinoma | significantly |
| 10: 1073 | | (127), squamous cell | differentially |
| (2001) | | carcinoma (21), | expressed between |
| | | pulmonary carcinoid (20), | NSCLC samples |
| | | small cell carcinoma (6), | and small cell |
| | | other adenocarcinomas | lung cancer and |
| | | (12), and normal lung | pulmonary carcinoid |
| | | (17) | samples; and |
| | | | Deriving |
| | | | normalization genes. |
| Acc. No. | U133 Plus | Frozen primary lung | Refining SQ/nSQ |
| GSE3141 | 2.0 | tumor samples (111): | gene set. |
| (Bild) | | Adenocarcinoma (58), | |
| | | squamous cell carcinoma | |
| | | (53) | |
| Acc. No. | U133 Plus | Frozen lung cancer | Refining SQ/nSQ |
| GSE2109 | 2.0 | samples (109): | gene set; and |
| (Bittner) | | Adenocarcinoma (39), | Deriving gene set |
| | | squamous cell carcinoma | significantly |
| | | (51), large cell carcinoma | differentially |
| | | (8) | expressed between |
| | | | colon-originating |
| | | | lung samples and |
| | | | NSCLC samples. |
| | | | Deriving |
| | | | normalization genes. |
| Acc. No. | U133 Plus | Snap-frozen lung tissue | Deriving |
| GSE12667 | 2.0 | samples (75): | normalization genes. |
| (Ding) | | Adenocarcinoma (68), | |
| | | squamous cell carcinoma | |
| | | (1) and large | |
| | | neuroendocrine (4) | |
| Acc. No. | U133 Plus | Snap-frozen lung tumor | Refining SQ/nSQ |
| GSE19188 | 2.0 | samples (91): | gene set; and |
| (Hou) | | Adenocarcinoma (45), | Deriving |
| | | squamous cell carcinoma | normalization genes. |
| | | (27), and large cell | |
| | | carcinoma (19) | |
| Acc. No. | U133 Plus | Frozen lung tumor | Refining SQ/nSQ |
| GSE8894 | 2.0 | samples (138): | gene set |
| (Kim) | | Adenocarcinoma (62), | |
| | | squamous cell carcinoma | |
| | | (76) | |
| Acc. No. | U133A | Frozen lung tumor | Refining SQ/nSQ |
| GSE14814 | | samples (90): | gene set |
| (Zhu) | | Adenocarcinoma (28), | |
| | | squamous cell carcinoma | |
| | | (52), and large cell | |
| | | carcinoma (10) | |
| Acc. No. | U133 Plus | Snap-frozen lung tumor | Refining SQ/nSQ |
| GSE10245 | 2.0 | samples (58): | gene set; and |
| (Kuner) | | Adenocarcinoma (18), | Deriving |
| | | squamous cell carcinoma | normalization genes. |
| | | (40) | |

¹Data sets described by the referenced Gene Expression Omnibus (GEO) Series Accession Nos. are those publicly available in the NCBI GEO database (Edgar et al., Nucleic Acids Res., 30(1): 207-10 (2002); Barrett et al., Nucleic Acids Res. 39: D1005-10 (2011)) as of Mar. 11, 2013.

Each publicly available “in silico” data set (i.e., all but Data Set 1) was analyzed independently with the Affymetrix Power Tool (APT) software package as previously described (Lockstone, Brief Bioinform., 12(6):634-44 (2011)). In silico data was normalized using quantile normalization and gene expression values determined by robust multi-array average (RMA) as described by Irizarry el al. (Biostatistics, 4:249 (2003)).

### Generation of Data Set 1

In addition to the publicly available data sets identified above, Data Set 1 was independently developed at least for the purpose of obtaining gene expression data using quantitative nuclease protection technology (qNPA). qNPA is an useful method for measuring gene expression in biological samples, and has particular advantages over other ex situ (e.g., “grind and bind”) methods (such as PCR), especially in fixed (e.g., FFPE) samples in which gene expression targets (e.g., RNA) may have degraded and are otherwise inaccessible.

Data Set 1 in combination with the in silico data sets provides a large, highly variable overall set of data for bioinformatic analysis. Such variability reduces various biases (e.g., platform, sample-type, and/or sample-preparation (pre-analytical) bias) that otherwise may affect the selection of genes useful for distinguishing squamous and nonsquamous subtypes of NSCLC and corresponding classifiers. Accordingly, the disclosed gene sets and NSCLC squamous/nonsquamous classifiers are robust and may be used with high confidence across pre-analytical conditions, gene expression methods and platforms.

### Preliminary Whole Transcriptome Analysis:

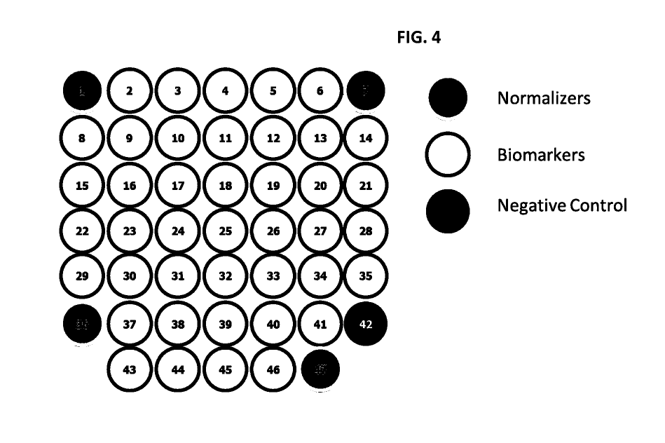

High-plex gene expression tests produce a large amount of data, which is useful for research and discovery purposes, but may overwhelm or be irrelevant especially for distributed clinical purposes. While there currently is no accepted maximum number of genes suitable for a clinically deployable gene expression test, currently available tests generally provide actionable data based on the expression of less than 100 genes (e.g., Mammaprint (70 genes), Oncotype Dx (21 genes)). One implementation of the qNPA technology, the 96×47 ArrayPlate (e.g., FIG. 4), is perfectly positioned in this mid-plex range because it measures the expression of up to 47 genes in up to 96 samples. To reduce transcriptome-level information to mid-plexity for qNPA implementation, a preliminary gene selection first was performed.

Nuclease protection assays (see below for additional detail) were conducted on a cohort of 134 FFPE lung samples, for which a histopathology-based diagnosis of NSCLC squamous cell carcinoma (70) or adenocarcinoma (64) was known. Recovered nuclease protection probes, which are surrogates for expressed RNAs, were detected on two custom arrays specific for 4600 mRNAs; 2600 of which were believed to be reasonably representative of the human transcriptome, and the remaining approximately 2000 of which were believed to be relevant to lung cancer survival.

Raw data were log 2 transformed, background subtracted and removed from further consideration if below a minimum relative light unit cut-off. A moderated t-test (LIMMA) was used to identify an initial list of genes significantly differentially expressed (p<0.05) between squamous cell carcinoma and adenocarcinoma samples. The initial gene list was further reduced by requiring at least a 1.5-fold expression difference between the sample types. Finally, genes (including genes not among the original 4600) were evaluated from the perspectives of pathway analysis and biological relevance and a subset of 126 candidate genes were selected for further study on three ArrayPlates, below.

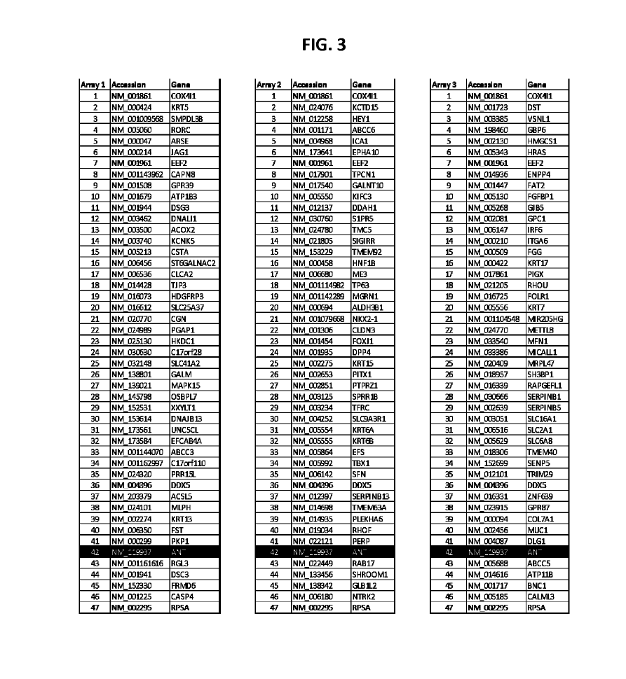

The 126 candidate genes are listed in Table 2 together with the relative expression of each gene in squamous (SQ) or nonsquamous (nonSQ) NSCLC samples. Exemplary GenBank Accession Nos. for these genes are shown in FIG. 3.

TABLE 2
Genes Differentially Expressed in Squamous (SQ) or Nonsquamous
(nonSQ) NSCLC Samples and Their Relative Expression.

| Gene Name | Relative Expression | Gene Name | Relative Expression |
|---|---|---|---|
| ZNF639 | SQ > nonSQ | UNC5CL | non-SQ > SQ |
| XXYLT1 | SQ > nonSQ | TPCN1 | non-SQ > SQ |
| VSNL1 | SQ > nonSQ | TMEM92 | non-SQ > SQ |
| TRIM29 | SQ > nonSQ | TMEM63A | non-SQ > SQ |
| TP63 | SQ > nonSQ | TMC5 | non-SQ > SQ |
| TMEM40 | SQ > nonSQ | TJP3 | non-SQ > SQ |
| TFRC | SQ > nonSQ | TBX1 | non-SQ > SQ |
| ST6GALNAC2 | SQ > nonSQ | SMPDL3B | non-SQ > SQ |
| SPRR1B | SQ > nonSQ | SLC41A2 | non-SQ > SQ |
| SLC9A3R1 | SQ > nonSQ | SLC25A37 | non-SQ > SQ |
| SLC6A8 | SQ > nonSQ | SIGIRR | non-SQ > SQ |
| SLC2A1 | SQ > nonSQ | SHROOM1 | non-SQ > SQ |
| SLC16A1 | SQ > nonSQ | SERPINB1 | non-SQ > SQ |
| SH3BP1 | SQ > nonSQ | RORC | non-SQ > SQ |
| SFN | SQ > nonSQ | RHOU | non-SQ > SQ |
| SERPINB5 | SQ > nonSQ | RHOF | non-SQ > SQ |
| SERPINB13 | SQ > nonSQ | RGL3 | non-SQ > SQ |
| SENP5 | SQ > nonSQ | RAB17 | non-SQ > SQ |
| S1PR5 | SQ > nonSQ | PRR15L | non-SQ > SQ |
| RAPGEFL1 | SQ > nonSQ | PLEKHA6 | non-SQ > SQ |
| PTPRZ1 | SQ > nonSQ | OSBPL7 | non-SQ > SQ |
| PKP1 | SQ > nonSQ | NKX2-1 | non-SQ > SQ |
| PITX1 | SQ > nonSQ | MUC1 | non-SQ > SQ |
| PIGX | SQ > nonSQ | MLPH | non-SQ > SQ |
| PGAP1 | SQ > nonSQ | MGRN1 | non-SQ > SQ |
| PERP | SQ > nonSQ | METTL8 | non-SQ > SQ |
| NTRK2 | SQ > nonSQ | ME3 | non-SQ > SQ |
| MRPL47 | SQ > nonSQ | MAPK15 | non-SQ > SQ |
| MIR205HG | SQ > nonSQ | KRT7 | non-SQ > SQ |
| MICALL1 | SQ > nonSQ | KRT15 | non-SQ > SQ |
| MFN1 | SQ > nonSQ | KIFC3 | non-SQ > SQ |
| KRT6B | SQ > nonSQ | KCNK5 | non-SQ > SQ |
| KRT6A | SQ > nonSQ | ICA1 | non-SQ > SQ |
| KRT5 | SQ > nonSQ | HNF1B | non-SQ > SQ |
| KRT17 | SQ > nonSQ | HKDC1 | non-SQ > SQ |
| KRT13 | SQ > nonSQ | GPR39 | non-SQ > SQ |
| KCTD15 | SQ > nonSQ | GLB1L2 | non-SQ > SQ |
| JAG1 | SQ > nonSQ | GALNT10 | non-SQ > SQ |
| ITGA6 | SQ > nonSQ | GALM | non-SQ > SQ |
| IRF6 | SQ > nonSQ | FST | non-SQ > SQ |
| HRAS | SQ > nonSQ | FOXJ1 | non-SQ > SQ |
| HMGCS1 | SQ > nonSQ | FOLR1 | non-SQ > SQ |
| HEY1 | SQ > nonSQ | FGG | non-SQ > SQ |
| HDGFRP3 | SQ > nonSQ | EPHA10 | non-SQ > SQ |
| GPR87 | SQ > nonSQ | ENPP4 | non-SQ > SQ |
| GPC1 | SQ > nonSQ | EFCAB4A | non-SQ > SQ |
| GJB5 | SQ > nonSQ | DPP4 | non-SQ > SQ |
| GBP6 | SQ > nonSQ | DNALI1 | non-SQ > SQ |
| FRMD6 | SQ > nonSQ | DNAJB13 | non-SQ > SQ |
| FGFBP1 | SQ > nonSQ | DDAH1 | non-SQ > SQ |
| FAT2 | SQ > nonSQ | CLDN3 | non-SQ > SQ |
| EFS | SQ > nonSQ | CGN | non-SQ > SQ |
| DST | SQ > nonSQ | CASP4 | non-SQ > SQ |
| DSG3 | SQ > nonSQ | CAPN8 | non-SQ > SQ |
| DSC3 | SQ > nonSQ | C17orf28 | non-SQ > SQ |
| DLG1 | SQ > nonSQ | C17orf110 | non-SQ > SQ |
| CSTA | SQ > nonSQ | ARSE | non-SQ > SQ |
| COL7A1 | SQ > nonSQ | ALDH3B1 | non-SQ > SQ |
| CLCA2 | SQ > nonSQ | ACSL5 | non-SQ > SQ |
| CALML3 | SQ > nonSQ | ACOX2 | non-SQ > SQ |
| BNC1 | SQ > nonSQ | ABCC6 | non-SQ > SQ |
| ATP1B3 | SQ > nonSQ | ABCC3 | non-SQ > SQ |
| ATP11B | SQ > nonSQ | | |
| ABCC5 | SQ > nonSQ | | |

### Expression of 126-Gene Subset in FFPE Lung Tissues:

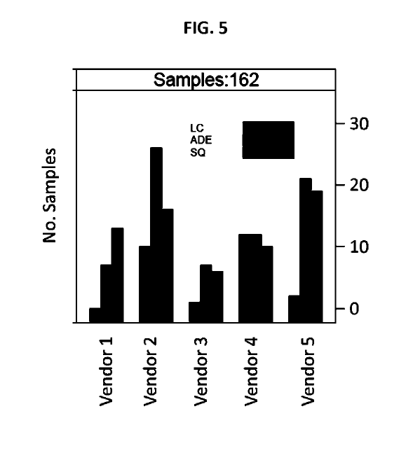

The expression of the 126 genes described above was determined in an independent cohort of 162 FFPE lung samples (adenocarcinoma (73), squamous cell carcinoma (64) and large cell carcinoma (25)) obtained from various commercial vendors (BioChain Institute, Inc. (Newark, Calif.), US Biomax, Inc. (Rockville, Md.), Cureline Inc. (South San Francisco, Calif.), Duke University (Durham, N.C.), ProteoGenex, Inc. (Culver City, Calif.)). The distribution of sample types by vendor is shown in FIG. 5.

### Sample Preparation and Lysis:

Briefly, each FFPE tissue section was measured to determine its approximate area (in cm²).

The tissue section then was scraped into a labeled eppendorf tube using a razor blade and avoiding any excess paraffin on the slide. The sample was suspended in 25 ul pre-warmed (50° C.) SSC buffer including formamide and SDS per each 0.3 cm² of the applicable tissue section. Five-hundred (500) ul of mineral oil containing a surfactant (e.g., Brij-97) (“Non-aqueous Layer”) then was overlaid on the tissue suspension, and this lysis reaction was incubated at 95° C. for 10-15 minutes. After briefly cooling the reaction mixture, proteinase K was added to a final concentration of 1 mg/ml and the incubation continued at 50 C for 30-60 minutes. A portion of the lysis reaction was used immediately in a nuclease protection assay (see below), or the lysis reaction (or remaining portion thereof) was frozen and stored at −80° C. Frozen lysis reactions were thawed at 50° C. for 10-15 minutes before a subsequent use.

### Nuclease Protection Assay (“NPA”):

Twenty-five (25) ul of each lysed reaction mixture was placed into each of three wells (triplicates) of a 96-well plate and overlaid with 70 ul Non-aqueous Layer. To each well was added 5 ul of nuclease protection probe (NPP) mix. One (1) nM (an excess of) NPP complementary to each of the plurality of RNA targets (e.g., mRNA and lncRNA) to be detected was present in the NPP mix. NPPs were (i) 50-base pairs in length with each half of the NPP having a Tm in the range of 40° C.-75° C. (and full length Tms in the range of 60° C.-85° C.) and (ii) tested in silico (using NCBI BLAST) and with in vitro transcripts for specificity to the respective RNA target (and substantially no cross-reactivity with other NPPs, other targets, or other analytes in the NPA reaction).

The 96-well NPA plate was heated at 95° C. for 10-15 minutes to denature nucleic acids and, then, allowed to incubate at 60° C. for 6-16 hours to permit hybridization of the NPPs to their respective RNA (e.g., mRNA and lncRNA) targets.

Following the hybridization step, 20 ul of excess S1 nuclease (2.5 U/ul) in sodium acetate buffer was added to the aqueous phase of each well. The S1 reaction proceeded at 50° C. for 90-120 minutes to digest unbound mRNA and unbound NPPs.

During the 51 digestion step, a 96-well “Stop” plate was prepared by adding 10 ul of solution contain 0.1 M EDTA and 1.6 N NaOH to each well corresponding to the reactions in the 96-well NPA plate. The entire volume (approx. 120 ul) of each reaction in the 96-well NPA plate was transferred to a corresponding well in the second 96-well Stop plate. The Stop plate was incubated at 95° C. for 15-20 minutes and, then, cooled for 5-10 minutes at room temperature prior to the addition of 10 ul 1.6 N HCl to neutralize the NaOH previously added to each reaction.

The nuclease protection assay reactions in this Example were interrogated directly (e.g., without purification or reverse transcription of target RNA analytes (e.g., mRNA and lncRNA)) using three 96-well-plate-based arrays (ArrayPlates) custom designed to detect in each well the expression of 42 of the candidate genes together with four normalizer (housekeeper) genes and a negative control. A listing of the genes detected on each of the three ArrayPlates and the respective gene's position on each array are shown in FIG. 3. Each well of an ArrayPlate contains an array of six rows of seven discrete sites (left to right: 1-7; 8-14; 15-21; 22-28; 29-35; 36-42) and a last row of 5 discrete sites (left to right: 43-47); a schematic diagram is shown in FIG. 4. The four normalizing (housekeeper) genes (indicated in gray in FIG. 3) were Cytochrome C Oxidase Subunit 4 Isoform 1, Mitochondrial (COX411), Eukaryotic Translation Elongation Factor 2 (EEF2), DEAD/H Box 5 (DDX5), and Laminin Receptor 1 (LAMR1; aka Ribosomal Protein SA (RPSA)), and Arabidopsis thaliana AP2-like ethylene-responsive transcription factor ANT was included as a negative control (indicated in black in FIG. 3).

### ArrayPlate Capture and Detection:

Each ArrayPlate was programmed with 40 ul 50-base pair programming linkers (“PL”) at 5 nM in SSC buffer containing SDS (“SSC-S”). The PLs were artificial, 25-base pair, bi-functional synthetic oligonucleotide constructs (adaptors) complementary in part to a universal anchor sequence affixed to the array surface and complementary in the other part to the particular NPP addressed to the particular array location. Following the programming step, the entire aqueous phase (60-65 ul) of each reaction from the Stop plate was added to a corresponding well of the programmed ArrayPlate and incubated at 50° C. for 16-24 hour to capture undigested NPPs (which were bound to target during the nuclease step and, therefore, are quantifiable surrogates for targets present in the sample). Thereafter, 5 nM bi-functional detection linker (“DL”) in SSC-S including 1% nonfat dry milk was added to each reaction followed by 1 hour incubation at 60° C. The DLs were artificial 25-base pair, bi-functional synthetic oligonucleotide constructs complementary in part to its respective NPP and complementary in the other part to one or more (e.g., two or three) copies of a biotin-labeled detection probe (“DP”), which DP was capable of specifically binding the detection-region designed into all DLs. To complete the detection “sandwich,” 40 ul of 3 nM DP was added to the reactions followed by 50° C. incubation for 45-60 min. Next, 40 ul avidin peroxidase (1:600) in SSC-S including 1% nonfat dry milk was added followed by incubation at 37° C. for 30-45 minute. Finally, a chemiluminescent substrate mix was added that, in the presence of peroxidase enzyme, generated light that was captured using a HTG OMIXTm imager. Gene expression is directly related to the intensity of light (relative light unit; RLU) emitted at each addressable position of the ArrayPlate.

### Data Pre-Processing:

The raw data was pre-processed as described in this subsection. Raw data was background subtracted and log 2 transformed. Any samples for which greater than 200 RLU was measured for the negative control gene, ANT, were deemed to have failed, and all data from those particular wells were removed from further consideration. A coefficient of variance (CV) was determined for replicate expression values for each gene. If the CV for sample replicates exceeded 6%, the replicate farthest from the average was removed as an outlier. Replicate reproducibility was measured by pairwise correlation and by pairwise simple linear regression. If the correlation had r >=0.90 and the intercept of the linear regression was not statistically significantly different from zero, such replicate was accepted; otherwise, it was deemed failed. Any sample with more than two failed replicates was defined as a failed sample.

### Example 2

### Gene Selection and NSCLC Squamous/Nonsquamous Classifier Development

This Example describes the selection of genes useful in the disclosed NSCLC squamous/nonsquamous classifiers from the data sets described in Example 1 and a representative 28-gene NSCLC squamous/nonsquamous classifier.

Multiple feature selection methods (RF, LIMMA, t-test, AUC) were used to evaluate whether a particular gene was significantly differentially expressed between sample types in each data set. Machine learning algorithms (e.g., Logistic Regression (LR), Random Forest (RF), Support Vector Machine (SVM), K-nearest neighbor (KNN)) were used to develop the initial classifier. Both feature selection and classification performance were evaluated in a leave-one-out cross-validated fashion. Error rate as a function of gene number and Receiver Operating Characteristic (ROC) curve were used to evaluate the performance of the classifier.

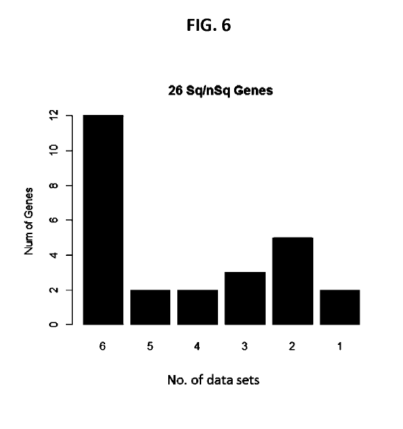

For purposes of 96×47 ArrayPlate implementation, approximately twenty-eight genes (or less) were determined to be preferred for a NSCLC squamous/nonsquamous classifier. This election leaves available 19 array sites per well for the co-detection of other genes of interest (e.g., positive and negative controls, and/or genes useful in other classifiers (e.g., pulmonary carcinoids or colon cancer lung metastases)). Many more than 28 genes were identified as significantly differentially expressed in all of the sample types of interest (e.g., squamous and nonsquamous NSCLC). Thus, genes sets were further refined by the repeatability of multiple gene selection methods as well as multiple data sets across different platforms with a preference also for genes from Dataset 1. Genes from Dataset 1 (see Table 2) that were repeatedly detected by multiple feature selection methods and/or that repeatedly appeared as significant across multiple data sets were given more weight in the selection of reduced gene sets. FIG. 6 shows that 24 of 26 genes from Dataset 1 were identified as significant differentially expressed in squamous and nonsquamous NSCLCs in two or more the independent analyses.

A selected set of 28 genes useful for distinguishing between squamous and nonsquamous NSCLC are listed in Table 3, as are the relative expression of each gene in squamous (SQ) or nonsquamous (nonSQ) NSCLC samples. Exemplary GenBank Accession Nos are provided.

TABLE 3
Genes Significantly Differentially Expressed between Squamous and Nonsquamous
NSCLC

| SQ/NonSQ | | | Relative |
|---|---|---|---|
| Gene Names | Description | GenBank Accession No(s). | Expression |
| CALML3 | Calmodulin-Like 3 (aka, | *NM_005185 (GI: 36031099) | SQ > nonSQ |
| | Calmodulin-Like Protein; | | |
| | CLP) | | |
| CLCA2 | Chloride Channel, Calcium- | *NM_006536 (GI: 187761335) | SQ > nonSQ |
| | Activated, 2 | | |
| CLDN3 | Claudin 3 (aka, Clostridium | *NM_001306 (GI: 171541813) | non-SQ > SQ |
| | Perfringens Enterotoxin | | |
| | Receptor 2; CPETR2; | | |
| | Clostridium Perfringens | | |
| | Enterotoxin Receptor, Low | | |
| | Affinity; Receptor of | | |
| | Enterotoxin of Clostridium | | |
| | Perfringens 2; Ventral | | |
| | Prostate 1, Rat, Homolog of; | | |
| | RVP1 Androgen Withdrawal | | |
| | Apoptosis Protein, Rat, | | |
| | Homolog of | | |
| CSTA | Cystatin A (aka, Stefin A; | *NM_005213 (GI: 61743964) | SQ > nonSQ |
| | STFA | | |
| | STF1) | | |
| DSC3 | Desmocollin 3 (aka, | *NM_001941 (GI: 148539845) | SQ > nonSQ |
| | Desmocollin 4; DSC4) | (variant Dsc3a); NM_024423 | |
| | | (GI: 148539847) (variant | |
| | | Dsc3b) | |
| DSG3 | Desmoglein 3 (aka, | *NM_001944 (GI: 119964717) | SQ > nonSQ |
| | Pemphigus Vulgaris Antigen; | | |
| | PVA) | | |
| KRT13 | Keratin 13 (aka, K13; | NM_153490 (GI: 131412224) | SQ > nonSQ |
| | Cytokeratin 13 | (variant 1); *NM_002274 | |
| | | (GI: 131412227) (variant 2) | |
| KRT5 | Keratin 5 (aka, K5) | *NM_000424 (GI: 119395753) | SQ > nonSQ |
| KRT6B | Keratin 6B (aka, Keratin, | *NM_005555 (GI: 119703752) | SQ > nonSQ |
| | Epidermal Type II; K6B) | | |
| PKP1 | Plakophilin 1 | NM_001005337 | SQ > nonSQ |
| | | (GI: 300068949) (variant la); | |
| | | *NM_000299 (GI: 300068950) | |
| | | (variant 1b) | |
| TP63 | Tumor Protein p63 (aka, | NM_003722 (GI: 169234655) | SQ > nonSQ |
| | Tumor Protein p73-Like; | (variant 1); NM_001114978 | |
| | TP73L; p53-Related Protein | (GI: 169234656) (variant 2); | |
| | p63; p63; KET | NM_001114979 | |
| | | (GI: 169234658) (variant 3); | |
| | | NM_001114980 | |
| | | (GI: 169234660) (variant 4); | |
| | | NM_001114981 | |
| | | (GI: 169234662) (variant 5); | |
| | | *NM_001114982 | |
| | | (GI: 169234664) (variant 6) | |
| TRIM29 | Tripartite Motif-Containing | *NM_012101 (GI: 109826574) | SQ > nonSQ |
| | Protein 29 (aka, Ataxia- | | |
| | Telangiectasia Group D- | | |
| | Associated Protein; ATDC) | | |
| KRT6A | Keratin 6A (aka, Keratin, | *NM_005554 (GI: 126273584) | SQ > nonSQ |
| | Epidermal Type II, K6A; | | |
| | K6A; K6C; K6D) | | |
| NKX2-1 | NK2 Homeobox 1 (aka, | *NM_001079668; | non-SQ > SQ |
| | Thyroid Transcription Factor | (GI: 261244895) (variant 1); | |
| | 1; TITF1; TTF1; Thyroid | NM_003317 (GI: 31881814) | |
| | Nuclear Factor; NK2, | (variant 2) | |
| | Drosophila, Homolog of, A; | | |
| | NKX2A; NK2.1, Mouse, | | |
| | Homolog of; Thyroid- | | |
| | Specific Enhancer-Binding | | |
| | Protein; TEBP | | |
| CAPN8 | Calpain 8 (aka, nCL-2; New | *NM_001143962 | non-SQ > SQ |
| | Calpain 2; Stomach-Specific | (GI: 221554548) | |
| | M-Type Calpain; NCL2) | | |
| SERPINB5 | Serpin Peptidase Inhibitor, | *NM_002639 (GI: 167860125) | SQ > nonSQ |
| | Clade B (Ovalbumin), | | |
| | Member 5 (aka, PI5; Maspin; | | |
| | Serine (or Cysteine) Protein | | |
| | Inhibitor, Clade B | | |
| | (Ovalbumin), Member 5; | | |
| | Peptidase Inhibitor 5; | | |
| | Protease Inhibitor 5 (Maspin); | | |
| | Serpin B5) | | |
| CGN | Cingulin (aka, Homolog of | *NM_020770 (GI: 187608600) | non-SQ > SQ |
| | Xenopus Cingulin; | | |
| | KIAA1319) | | |
| MUC1 | Mucin 1, Transmembrane | *NM_002456 (GI: 324120948) | non-SQ > SQ |
| | (aka, Mucin 1, Urinary; | (variant 1); NM_001018016 | |
| | Peanut-Reactive Urinary | (GI: 324120953) (variant 2); | |
| | Mucin; PUM; Tumor- | NM_001018017 | |
| | Associated Epithelial Mucin; | (GI: 324120954) (variant 3); | |
| | Polymorphic Epithelial | NM_001044390 | |
| | Mucin; PEM; Epithelial | (GI: 324120952) (variant 5): | |
| | Membrane Antigen; EMA | NM_001044391 | |
| | | (GI: 324120951) (variant 6); | |
| | | NM_001044392 | |
| | | (GI: 324120950) (variant 7); | |
| | | NM_001044393 | |
| | | (GI: 324120949) (variant 8); | |
| | | NM_001204285 | |
| | | (GI: 324120955) (variant 9); | |
| | | NM_001204286 | |
| | | (GI: 324120957) (variant 10); | |
| | | NM_001204287 | |
| | | (GI: 324120959) (variant 11); | |
| | | NM_001204288 | |
| | | (GI: 324120961) (variant 12); | |
| | | NM_001204289 | |
| | | (GI: 324120963) (variant 13) | |
| | | NM_001204290 | |
| | | (GI: 324120965) (variant 14); | |
| | | NM_001204291 | |
| | | (GI: 324120967) (variant 15); | |
| | | NM_001204292.1 | |
| | | (GI: 324120969) (variant 16); | |
| | | NM_001204293 | |
| | | (GI: 324120971) (variant 17); | |
| | | NM_001204294 | |
| | | (GI: 324120973) (variant 18); | |
| | | NM_001204295 | |
| | | (GI: 324120975) (variant 19); | |
| | | NM_001204296 | |
| | | (GI: 324120979) (variant 20); | |
| | | NM_001204297 | |
| | | (GI: 324120977) (variant 21); | |
| PERP | p53 Effector Related to | *NM_022121 (GI: 222080101) | SQ > nonSQ |
| | PMP22 (aka, THW) | | |
| IRF6 | Interferon Regulatory Factor | *NM_006147 (GI: 331999973) | SQ > nonSQ |
| | 6 | (variant 1); NM_001206696 | |
| | | (GI: 331999977) (variant 2) | |
| KCNK5 | Potassium Channel, | *NM_003740 (GI: 88999598) | non-SQ > SQ |
| | Subfamily K, Member 5 (aka, | | |
| | TASK2) | | |
| SLC2A1 | Solute Carrier Family 2 | *NM_006516 (GI: 166795298) | SQ > nonSQ |
| | (Facilitated Glucose | | |
| | Transporter), Member 1 (aka, | | |
| | Glucose Transporter 1; | | |
| | GLUT; GLUT1; | | |
| | Erythrocyte/Hepatoma | | |
| | Glucose Transporter) | | |
| TJP3 | Tight Junction Protein 3 | NM_001267560 | non-SQ > SQ |
| | | (GI: 389565492) (variant 1); | |
| | | NM_001267561 | |
| | | (GI: 389565500) (variant 2); | |
| | | *NM_014428 (GI: 10092690) | |
| | | (variant not specified) | |
| KRT7 | Keratin 7 (aka, K7; Keratin, | *NM_005556 (GI: 67782364) | non-SQ > SQ |
| | Simple Epithelial; Keratin, | | |
| | Type II, Cytoskeletal, 7; | | |
| | K2C7; Sarcolectin; SCL) | | |
| MIR205HG | MIR205 host gene (non- | *NM_001104548 | SQ > nonSQ |
| | protein coding) (aka, | (GI: 157151758) | |
| | LINC00510) | | |
| RGL3 | Ral Guanine Nucleotide | *NM_001161616 | non-SQ > SQ |
| | Dissociation Stimulator-Like | (GI: 239582764) (variant 1); | |
| | 3 (aka, Ra1GDS-like; | NM_001035223 | |
| | FLJ32585; RalGEF-like | (GI: 239582762) (variant 2) | |
| | protein 3, Mouse Homolog | | |
| TP63 (ΔNp63- | Tumor Protein p63 | *NM_001114980 | SQ > nonSQ |
| encoding | | (GI: 169234660) (variant 4); | |
| variants) | | NM_001114981 | |
| | | (GI: 169234662) (variant 5); | |
| | | NM_001114982 | |
| | | (GI: 169234664) (variant 6) | |
| S100A2 | S100 calcium binding protein | *NM_005978 (GI: 45269153) | SQ > nonSQ |
| | A2 (aka, S100L; CAN19) | | |

*Representative sequence used in probe design.

When applied to the cohort of 162 FFPE lung samples, the validation accuracy of a representative support vector machine (SVM) classifier based on the foregoing genes yielded a ROC AUC (area under the curve) of 0.98, and a NSCLC subtype classification accuracy of 95.1% (using a cutoff value of p<=0.05). When applied to the “discovery” cohort of 134 lung samples from Data Set 1 (see Example 1), this representative SVM classifier yielded a ROC AUC of 0.951, and correctly subtyped 121 of such 134 samples as squamous or nonsquamous for an accuracy of 90.3%. Because the discovery cohort was not independently adjudicated by an expert panel of pathologists (as were the 162-sample and 97-sample cohorts), it is believed that the apparent decreased in the accuracy of the representative classifier when applied to the discovery cohort is not, in fact, in all cases, incorrect classification of the sample by the classifier, but mislabeling of the sample (e.g., misclassified sample is labeled squamous when it is, in fact, a nonsquamous NSCLC or vice versa). Thus, in some examples the disclosed methods have an accuracy of at least 90%, at least 91%, at least 92%, at least 93%, at least 94%, or at least 95%.

As discussed previously, many more genes significantly differentially expressed in squamous and nonsquamous NSCLC were identified than were selected (merely for technical convenience) for ArrayPlate implementation. An ordinarily skilled artisan will appreciate that subsets of the 28 genes in Table 3 can be useful for subtyping squamous and nonsquamous NSCLCs and for use in NSCLC squamous/nonsquamous classifiers. In addition, other genes significantly differentially expressed in squamous and nonsquamous NSCLC subtypes identified by the present analysis also are useful for such purposes. Additional such genes that were significantly differentially expressed in six of the squamous/nonsquamous NSCLC data sets are shown in Table 4, as is the relative expression of each gene in squamous (SQ) or nonsquamous (nonSQ) NSCLC samples. Exemplary GenBank Accession Nos are provided.

TABLE 4
Additional Genes Significantly Differentially Expressed between
Squamous and Nonsquamous NSCLC

| | | GenBank | |
|---|---|---|---|
| SQ/NonSQ | | Accession | Relative |
| Gene Names | Description | No(s). | Expression |
| DST | Dystonin (aka, Homolog | NM_001723 | SQ > |
| | of Mouse Dystonia | (GI: 291290966) | nonSQ |
| | Musculorum; DMH; DT; | (variant 1e); | |
| | Bullous Pemphigoid | NM_015548 | |
| | Antigen 1; PAG1; BP240) | (GI: 291290967) | |
| | | (variant 1eA) | |
| KRT17 | Keratin 17 (aka, K17; | NM_000422 | SQ > |
| | Cytokeratin 17) | (GI: 197383031) | nonSQ |
| NTRK2 | Neurotrophic Tyrosine | NM_006180 | SQ > |
| | Kinase, Receptor, Type 2 | (GI: 65506645) | nonSQ |
| | (aka, Tyrosine Kinase | (variant a); | |
| | Receptor B; TRKB) | NM_001007097 | |
| | | (GI: 55956789) | |
| | | (variant b); | |
| | | NM_001018064 | |
| | | (GI: 65506744) | |
| | | (variant c); | |
| | | NM_001018065 | |
| | | (GI: 206597532) | |
| | | (variant d); | |
| | | NM_001018066 | |
| | | (GI: 206597545) | |
| | | (variant e) | |
| PI13 (aka | Protease Inhibitor 13 (aka, | NM_012397 | SQ > |
| SERPINB13) | Serine Protease Inhibitor, | (GI: 196259794) | nonSQ |
| | Clade B, Member 13; | | |
| | Hurpin; Headpin) | | |
| SLC6A8 | Solute Carrier Family 6 | NM_005629 | SQ > |
| | (Neurotransmitter | (GI: 218563759) | nonSQ |
| | Transporter, Creatine), | (variant 1); | |
| | Member 8 (aka, Creatine | NM_001142805 | |
| | Transporter; CT1; CRTR; | (GI: 218563755) | |
| | CRT) | (variant 2); | |
| | | NM_001142806 | |
| | | (GI: 218563757) | |
| | | (variant 3) | |
| SPRR1A | Small Proline-Rich Protein | NM_001199828 | SQ > |
| | 1A | (GI: 315360634) | nonSQ |
| | | (variant 1); | |
| | | NM_005987 | |
| | | (GI: 315360633) | |
| | | (variant 2) | |
| SPRR1B | Small Proline-Rich Protein | NM_003125 | SQ > |
| | 1B | (GI: 83582814) | nonSQ |
| SPRR3 | Small Proline-Rich Protein | NM_005416.2 | SQ > |
| | 3 | (GI: 147905322) | nonSQ |
| | | (variant 1); | |
| | | NM_001097589 | |
| | | (GI: 148229143) | |
| | | (variant 2) | |

### Example 3

### Molecular Identification of Lung Tumor Samples Misidentified as NSCLC

Classifying NSCLC samples as squamous and nonsquamous subtypes is advantaged by the proper identification of the input samples as NSCLC, typically by histology or IHC. Certain lung tumor samples (e.g., lung metastases of primary colon cancers, small cell lung carcinomas, and pulmonary carcinoids) may be misdiagnosed as NSCLC using ordinary clinical methods. Disclosed gene expression studies revealed clusters other than squamous and nonsquamous NSCLC, and gene sets and corresponding classifiers were developed to identify these misdiagnosed lung tumor samples. These innovations may stand on their own as classifiers or, optionally, may be used together with disclosed NSCLC squamous/nonsquamous classifiers, e.g., to identify and remove from the NSCLC squamous/nonsquamous analysis any non-NSCLC (e.g., colon metastases, small cell, or carcinoids) lung samples.

### Lung Metastases of Primary Colon Tumors

This section describes gene sets and classifiers useful to identify a lung sample as a metastasis from a primary colon tumor, or to identify colon metastases that have been misdiagnosed as NSCLC and, in particular embodiments, to remove from consideration or treat as “indeterminant” such misdiagnosed samples when using a disclosed NSCLC squamous/nonsquamous classifier (Table 5). Bioinformatic analyses were similar to those described in the foregoing Examples.

TABLE 5
Genes Significantly Differentially Expressed between Colon-
Originating and Lung-Originating Samples

| Colon | | GenBank | |
|---|---|---|---|
| Met Gene | | Accession | Relative |
| Names | Description | No(s). | Expression |
| *SFTPB | Surfactant Protein B (aka, | *NM_000542 | NSCLC > |
| | SFTP3; Surfactant, | (GI: 288856298) | Colon |
| | Pulmonary-Associated | (variant 1); | |
| | Protein B; SP-B; SFTB3; | NM_198843 | |
| | SMDP1; SPS-B) | (GI: 288856296) | |
| | | (variant 2) | |
| *CLRN3 | Clarin 3 (aka, Transmembrane | *NM_152311 | Colon > |
| | Protein 12; TMEM12; | (GI: 219521912) | NSCLC |
| | USH3AL1; MGC32871; | | |
| | Usher Syndrome Type- | | |
| | 3A-Like Protein) | | |
| *CDH17 | Cadherin 17 (aka, LI Cadherin | *NM_004063 | Colon > |
| | (liver-intestine); HPT-1; | (GI: 221316592) | NSCLC |
| | CDH16; Cadherin; HPT-1 | (variant 1); | |
| | Cadherin; Human Peptide | NM_001144663 | |
| | Transporter 1) | (GI: 221316594) | |
| | | (variant 2) | |
| *LGALS4 | Lectin, Galactoside-Binding, | *NM_006149 | Colon > |
| | Soluble, 4 (aka, GAL4; | (GI: 194578913) | NSCLC |
| | L36LBP; Galectin 4; L-36; | | |
| | Lactose Binding Protein) | | |
| *CXCL17 | Chemokine (C-X-C Motif) | *NM_198477 | Colon > |
| | Ligand 17 (aka, DMC; VCC1; | (GI: 38348269) | NSCLC |
| | Dcip1; VEGF Co-Regulated | | |
| | Chemokine) | | |
| SFTPA2 | Surfactant, Pulmonary- | NM_001098668 | NSCLC > |
| | Associated Protein A2 (aka, | (GI: 257743448) | Colon |
| | Pulmonary Surfactant Protein | | |
| | AII; SPAII; SPA2 | | |
| | Collectin 5; COLEC5) | | |
| SCGB3A2 | Secretoglobin, Family 3A, | NM_054023 | NSCLC > |
| | Member 2 (aka, Uteroglobin- | (GI: 290463439) | Colon |
| | Related Protein 1; UGRP1) | | |
| NAPSA | Napsin A (aka, Pronapsin A; | NM_004851 | NSCLC > |
| | NAPA; NAP1) | (GI: 4758753) | Colon |
| SFTPD | Surfactant, Pulmonary- | NM_003019 | NSCLC > |
| | Associated Protein D (aka, | (GI: 61699225) | Colon |
| | Pulmonary Surfactant | | |
| | Apoprotein PSP-D; PSP-D | | |
| | Surfactant Protein D; SP-D; | | |
| | Surfactant-Associated Protein, | | |
| | Pulmonary 4; SFTP4; | | |
| | Collectin 7; COLEC7) | | |
| AQP4 | Aquaporin 4 (aka, Mercurial- | NM_001650 | NSCLC > |
| | Insensitive Water Channel; | (GI: 50659061) | Colon |
| | MIWC) | (variant a); | |
| | | NM_004028 | |
| | | (GI: 50659062) | |
| | | (variant b) | |
| SFTA3 | Surfactant Associated 3 (aka, | NM_001101341 | NSCLC > |
| | Surfactant Associated Protein | (GI: 157412272) | Colon |
| | H; SFTPH; Putative Protein | | |
| | SFTA) | | |
| SFTPC^{†} | Surfactant, Pulmonary- | NM_003018 | NSCLC > |
| | Associated Protein C (aka, | (GI: 149999607) | Colon |
| | Surfactant-Associated Protein, | (variant 1); | |
| | Pulmonary, 2; SFTP2; | NM_001172410 | |
| | Pulmonary Surfactant | (GI: 288915520) | |
| | Apoprotein PSP-C; SPC; PSP- | (variant 2); | |
| | C; Surfactant Proteolipid SPL- | NM_001172357 | |
| | pVal; Pulmonary Surfactant | (GI: 288915542) | |
| | Protein SP5 | (variant 3) | |
| CP^{†} | Ceruloplasmin (aka, | NM_000096 | NSCLC > |
| | Ferroxidase) | (GI: 189458860) | Colon |
| | | (variant 1); | |
| | | NR_046371 | |
| | | (GI: 377823734) | |
| | | (variant 2) | |
| MUC13^{†} | Mucin 13, Cell Surface- | NM_033049 | Colon > |
| | Associated | (GI: 308736984) | NSCLC |
| HEPH | Hephaestin | NM_138737 | Colon > |
| | | (GI: 281485617) | NSCLC |
| | | (variant 1); | |
| | | NM_014799 | |
| | | (GI: 21166383) | |
| | | (variant 2); | |
| | | NM_001130860 | |
| | | (GI: 281485623) | |
| | | (variant 3) | |
| ZNF512B | Zinc Finger Protein 512B | NM_020713 | NSCLC > |
| | (aka GM632) | (GI: 444741675) | Colon |
| USH1C^{†} | USH1C Gene (aka, Hamonin, | NM_005709 | NSCLC > |
| | PDZ Domain-Containing | (GI: 225690577) | Colon |
| | Protein, 73-kD; PDZ73) | (variant 1); | |
| | | NM_153676 | |
| | | (GI: 225703075) | |
| | | (variant b3) | |

*Representative sequence used in probe design.
^{†}Significant differential expression (lung v. colon) based on multiple different probes (note, however, not all targets were detected with multiple probes)

### Identifying the Group of Pulmonary Carcinoids and Small Cell Lung Cancer

This section describes gene sets and classifiers useful to identify a lung sample as belonging to the group of pulmonary carcinoids and small cell lung cancers, or to identify pulmonary carcinoids and small cell lung cancers that have been misidentified as NSCLC using other methods (e.g., histology or IHC) and, in particular embodiments, to remove from consideration or treat as “indeterminant” such misidentified samples when using a disclosed NSCLC squamous/nonsquamous classifier (Table 6). Bioinformatic analyses were similar to those described in the foregoing Examples.

TABLE 6
Genes Significantly Differentially Expressed Between Pulmonary Carcinoid (PCN) and
Small Cell Lung Cancer (SMC) Samples and NSCLC Samples

| Carcinoid | | | |
|---|---|---|---|
| and Small | | | |
| Cell Gene | | GenBank Accession | |
| Names | Description | No(s). | Relative Expression |
| *CHGA | Chromogranin A | *NM_001275 | PCN/SMC > NSCLC |
| | (parathyroid secretory | (GI: 134244286) | |
| | protein 1) (aka, Pituitary | | |
| | Secretory Protein 1; SP-1; | | |
| | CGA; Betagranin; CgA) | | |
| *TSPYL2 | TSPY-Like 2 (aka, CDA1; | *NM_022117 | PCN/SMC > NSCLC |
| | DENTT; CINAP; CTCL; | (GI: 259906401) | |
| | Cell Division Autoantigen; | | |
| | Cutaneous T-Cell | | |
| | Lymphoma-Associated | | |
| | Antigen se20-4; NP79; | | |
| | TSPX; SE20-4; Testis- | | |
| | Specific Protein Y | | |
| | Encoded-Like 2) | | |
| *APLP1 | Amyloid Beta (A4) | NM_001024807 | PCN/SMC > NSCLC |
| | Precursor-Like Protein 1 | (GI: 67782337) | |
| | (aka, APLP) | (variant 1); | |
| | | *NM_005166 | |
| | | (GI: 67782339) | |
| | | (variant 2) | |
| *CAMK2B | Calcium/Calmodulin- | *NM_001220 | PCN/SMC > NSCLC |
| | Dependent Protein Kinase II | (GI: 212549591) | |
| | Beta (aka, CAM2; | (variant 1); | |
| | CAMKB; CaMK-II Subunit | NM_172078 | |
| | Beta) | (GI: 212549588) | |
| | | (variant 2); | |
| | | NM_172079 | |
| | | (GI: 212549583) | |
| | | (variant 3); | |
| | | NM_172080 | |
| | | (GI: 212549590) | |
| | | (variant 4); | |
| | | NM_172081 | |
| | | (GI: 212549585) | |
| | | (variant 5); | |
| | | NM_172082 | |
| | | (GI: 212549589) | |
| | | (variant 6); | |
| | | NM_172083 | |
| | | (GI: 212549587) | |
| | | (variant 7); | |
| | | NM_172084 | |
| | | (GI: 212549586) | |
| | | (variant 8) | |
| *TAGLN3 | Transgelin 3 (aka, NP22; | *NM_013259 | PCN/SMC > NSCLC |
| | NP25; Neuronal Protein 22; | (GI: 56549134) | |
| | Neuronal Protein NP25) | (variant 1); | |
| | | NM_001008272 | |
| | | (GI: 56549136) | |
| | | (variant 2); | |
| | | NM_001008273 | |
| | | (GI: 56549138) | |
| | | (variant 3) | |
| *NCAM1 | Neural Cell Adhesion | *NM_000615 | CCN/SMC > NSCLC |
| | Molecule 1 (aka, CD56; | (GI: 336285433) | |
| | Antigen MSK39 Identified | (variant 1); | |
| | by Monoclonal Antibody | NM_181351 | |
| | 5.1H11; MSK39) | (GI: 336285435) | |
| | | (variant 2); | |
| | | NM_001076682 | |
| | | (GI: 336285437) | |
| | | (variant 3); | |
| | | NM_001242608 | |
| | | (GI: 336285442) | |
| | | (variant 4); | |
| | | NM_001242607 | |
| | | (GI: 336285439) | |
| | | (variant 5) | |

*Representative sequence used in probe design.

### Example 4

### Housekeeper Selection for Lung Malignancy Gene Expression Data

Disclosed classifiers are advantaged by means to account for sample-to-sample variations, such as difference in sample load. Various means are well known to the ordinarily skilled artisan and all such means are contemplated by this disclosure. One representative and common method of sample-to-sample control is to co-detect in each sample the expression of one or more “housekeeper” gene, the expression of which is statistically non-variant across samples.

The former scientific dogma that certain genes have constant expression across all sample types (i.e., universal “housekeeper” gene) has lost favor (e.g., Avison, Measuring Gene Expression, Psychology Press, 2007, p. 128). Thus, other alternatives for selecting genes suitable for normalization, especially, of microarray data have been developed. This Example describes the robust process that was used to identify genes whose expression was consistent (i.e, had low variability) across all lung sample types (including colon metastases to the lung) that are likely to be tested by disclosed classifiers.

In silico meta-data mining was performed using expression data from thousands of relevant samples (e.g., NSCLC, colon, and lung) from many different array data sets. Each data set was separately quartile normalized, log transformed and background subtracted. Gene data was filtered out if mean intensity was greater than the max(intensity)-2*SD or the maximum fold change within a data set was =>2. Data passing these filters were ranked by CV in ascending order. The Eucleadian distance of the ranks across multiple data sets was used as a mean score for measuring the rank of gene invariance across many data sets and biological conditions. Candidate genes that had significant difference among subtypes of interest (e.g., SQ/nonSQ, colon/NSCLC, PCN/SMC/NSCLC) were removed. Genes that were potential cross-hybridizers and/or saturated were down weighted in the final selection.

Eleven representative genes useful for normalizing across lung malignancy samples (e.g., samples diagnosed by IHC or histopathology methods as NSCLC) are listed in Table 7; such genes are referred to as housekeepers or normalizing genes or normalizers or endogenous controls:

TABLE 7
Housekeeping/Normalizing genes

| Gene Name | Gene Description | RNA RefSeq Accession No(s). |
|---|---|---|
| *EEF2 | Eukaryotic Translation Elongation Factor 2 | NM_001961 (GI: 83656775) |
| | (aka, Elongation Factor 2 or Polypeptidyl- | |
| | tRNA Translocase) | |
| *RPSA | Laminin Receptor 1 (aka, LAMR1, | NM_002295 (GI: 70609879) |
| | LAMBR, Ribosomal Protein SA, Laminin | (variant 1); NM_001012321 |
| | Receptor, 67-KD, or 67LR) | (GI: 59859884) (variant 2) |
| *DDX17 | DEAD/H BOX 17 (aka, RNA Helicase, | NM_006386 (GI: 148613854) |
| | 70-KD; RH70; p72) | (variant 1); NM_001098504 |
| | | (GI: 148613855) (variant 2) |
| *HMGXB3 | HMG box domain containing 3 (aka, | NM_014983 (GI: 241982801) |
| | HMGX3; SMF; KIA0194) | |
| *RPL19 | Ribosomal Protein L19 | NM_000981 (GI: 68216257) |
| *RPS29 | Ribosomal Protein S29 | NM_001032 (GI: 71772593) |
| | | (variant 1); NM_001030001 |
| | | (GI: 71772582) (variant 2) |
| RPL37A | Ribosomal protein L37A | NM_000998 (GI: 78214519) |
| RPL41 | Ribosomal protein L41 (aka, HG12) | NM_021104 (GI: 10863874) |
| | | (variant 1); NM_001035267 |
| | | (GI: 78217383) (variant 2) |
| CFL1 | Cofilin 1 (non-muscle) | NM_005507 (GI: 49472823) |
| MT-ND4 | NADH dehydrogenase 4 (E.C. 1.6.5.3) | AF253979 |
| OAZ1 | Ornithine Decarboxylase Antizyme 1 | D87914 (GI: 1590807); |
| | | NM_004152 (GI: 34486089) |
| | | (RefSeq) |

*Included on verification array

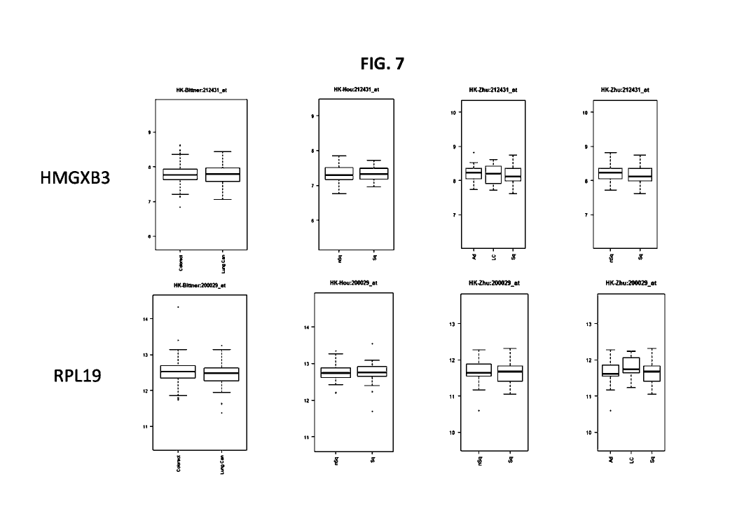

FIG. 7 shows representative box and whisker plots for HMGXB3 and RPL19 compared among the sample types indicated on the x-axes. These data show that there is no significant difference in the expression of these two genes in a variety of lung and colon samples. Similar results were obtained for each gene in Table 7. Accordingly, at least the genes in Table 7 (or any one or a subset thereof) may serve as useful normalizers for samples (e.g., tissues or cells) originating from lung and colon.

In any given set of samples (whether of the same or different cell or tissue origin), many genes whose expression is not significantly different across such sample population (aka, housekeepers, normalizers, endogenous controls or the like) may be identified using the methods described in this Example, or any other of known to those of ordinary skill in the art. Many more genes, whose expression was statistically invariant across samples likely to be tested in the disclosed methods, were identified than were selected (merely for technical convenience) for ArrayPlate implementation.

### Example 5

### Validation of NSCLC Squamous/Nonsquamous Classifier

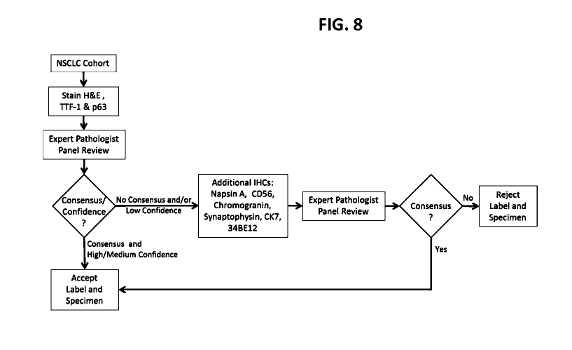

A representative NSCLC squamous/nonsquamous classifier was verified in an independent cohort of 97 samples. The samples were obtained from a variety of commercial and other sources with the aim to mimic the heterogeneity expected in NSCLC sample collection and fixation methods one might expect to see at a community hospital. The specimen subtypes consisted of squamous carcinoma, adenocarcinoma, and other nonsquamous, non-adenocarcinoma NSCLC subtypes. Consensus reads from a panel of expert pathologists were used to assign specimen tumor classification labels as shown in FIG. 8.

Expression of the genes shown in Table 8 was determined in cohort samples using the qNPA methods described in Example 1. The array positions (“Pos.”) and representative nuclease protection probes also are provided in Table 8. It is understood that the target sequence to which the NPP sequence specifically binds is the reverse complement of the NPP sequence shown.

TABLE 8
Verification Array Genes and Nuclease Protection Probes

| | | | | SEQ |
|---|---|---|---|---|
| | | | NPP Sequence | ID |
| Pos. | Accession No. | Identifier | (5′ to 3′ wrapped at right as needed) | NO. |
| 1 | NC_015216 | Pos.Control | CTGCCAAGTAATTCGCAGGCTTCTTCGGCC | 1 |
| | | | TTATTTAACAAGGAGTGCTG | |
| 2 | NM_000424 | KRT5 | GGCAGTGACTTGCAGCAGGTTCTTAGCTCT | 2 |
| | | | TGAAGCTCTTCCGGGAGGAG | |
| 3 | NM_000542 | SFTPB | GAAGAAAGCTTGCCCGGTCGCCATCCCATC | 3 |
| | | | ATGCCAGAGCGTGCAGTGTC | |
| 4 | NM_152311 | CLRN3 | CAAATGCTGTCACAGATGACAGTCACGTTA | 4 |
| | | | CCATGCTGAATTGTCCAGAG | |
| 5 | NM_004063 | CDH17 | ATAAATGGAATCCAGGCAGTTCTATGAGAC | 5 |
| | | | AACACTGATATATCTCCTTG | |
| 6 | NM_006149 | LGALS4 | CCAAGCCACAGCGAATGGACAGATCAAAGA | 6 |
| | | | ACTGTCCGGGACCAAATGGG | |
| 7 | NM_001961 | EEF2 | CTTGATGGTGATGCAACGCTCCTGCTCGTC | 7 |
| | | | CTTCCGGGTATCAGTGAAGC | |
| 8 | NM_001143962 | CAPN8 | GAACCTGGTAGACGGCATAGCCGATGCTAA | 8 |
| | | | GCATGCCTTGTCCTATCCGC | |
| 9 | NM_001275 | CHGA | CAATGGCCGACAGGCTCTCCAGCTCCTGGT | 9 |
| | | | CCTCTGGTCTGCGGTTTGCG | |
| 10 | NM_022117 | TSPYL2 | GATGTCCTCATCTTGCTGGATGCCTTCCTC | 10 |
| | | | AATGCCCTCTTCTTCCACTG | |
| 11 | NM_001944 | DSG3 | GGAACCTCCTCAGGACTTACCAGGCTGCTG | 11 |
| | | | CACAGGATCTAGCTTCTCCC | |
| 12 | NM_005166 | APLP1 | GCTCATCCCTCTGAATCTCCGATGAGTGGA | 12 |
| | | | AAGGGAAACCCCTTGGAACA | |
| 13 | NM_006147 | IRF6 | GACAAGCAGGTTCGTGCTAGGTGAGCCTTT | 13 |
| | | | CCAGAAGAGTACTGCCCAGC | |
| 14 | NM_003740 | KCNK5 | GATACGTGGCCTTTGCTCCTCAGTGTCTGT | 14 |
| | | | GACACCTCTTCCAAGGTGGG | |
| 15 | NM_005213 | CSTA | CATGACTCAGTAGCCAGTTGAAGGAATCAG | 15 |
| | | | AACACTTTGGGTACATGCTG | |
| 16 | NM_001220 | CAMK2B | CCCTTCAACCAGGTTGCCCAGTGCTTCAGG | 16 |
| | | | CTCAAACGAGGTCAGCCCTG | |
| 17 | NM_006536 | CLCA2 | CAGCCTGTACAAGCGAGAATGTGGGAGGAG | 1 |
| | | | GTGGAAGCTCAGTCCCATTC | |
| 18 | NM_014428 | TJP3 | GCACCAGCAGGCTTAGCTTCCCTTCTGACT | 18 |
| | | | TCTCAATCAGTCGCCGGGTG | |
| 19 | NM_001114982 | TP63 | CACTAGTGGCTTTGTGCCTTTGAGCAGTTG | 19 |
| | | | GGTCTCTGAGCCAAAGTGTC | |
| 20 | NM_005556 | KRT7 | CCACAGATGTGTCGGAGATCTGGGACTGCA | 20 |
| | | | GCTCTGTCAACTCCGTCTCA | |
| 21 | NM_001104548 | MIR205HG | TTTCCAATCTGCCCATCACCCGTCCCTCTG | 21 |
| | | | AAGAAGCACGCACACTCCAG | |
| 22 | NM_001306 | CLDN3 | CTGACTCACCGACGGCGCGCGCTAACGGCT | 22 |
| | | | CGGCTCCATACGCTCTCGCC | |
| 23 | NM_020770 | CGN | CCTTCACCCTCAGGCTTAGCTGGTCTTTCT | 23 |
| | | | GGTCATTGACATGCTGCCGC | |
| 24 | NM_013259 | TAGLN3 | AGTTCTAAATGAACAGGAGGTGGCAGCAGT | 24 |
| | | | AGGCAACGCGGATTCTCGGC | |
| 25 | NM_001079668 | NKX2-1 | TTACAAGCGAGTCCTCTTTGCTGGCAGAGT | 25 |
| | | | GTGCCCAGAGTGAAGTTTGG | |
| 26 | NM_000615 | NCAM1 | CTATTCTGAGGGCCTGTGCATTTGAACCAG | 26 |
| | | | AGATCTGTGCAGGCCCTAGA | |
| 27 | NM_006386 | DDX17 | CAGCCCATTGCTTAATACATTGGAACCCTT | 27 |
| | | | TCCCTAAGTTGAGTTTCAAC | |
| 28 | NM_014983 | HMGXB3 | GGAAGAGCAAGAGAGAAATGCATCCCTCCT | 28 |
| | | | TCAGGGAGAATCAAGAGCCC | |
| 29 | NM_002639 | SERPINB5 | CATTTGCAGTGTCACCTTTAGCACCCACTT | 29 |
| | | | GAGCAAGTGACAGAGAGGTG | |
| 30 | NM_001032 | RPS29 | CGGAAACACTGGCGGCACATATTGAGGCCA | 30 |
| | | | TATTTCCGGATCAGACCGTG | |
| 31 | NM_006516 | SLC2A1 | CAGGACCCACTTCAAAGAAGGCCACAAAGC | 31 |
| | | | CAAAGATGGCCACGATGCTC | |
| 32 | NM_005555 | KRT6B | GAATGCAGACTGCATCAGAAGGTACATCAC | 32 |
| | | | TTGCCATTCAGGGACACTGC | |
| 33 | NM_005554 | KRT6A | GAAGGTGAGCTTGCAGGTTGGGAAGGGCTG | 33 |
| | | | GGCTTTACCAACAGTGAGAT | |
| 34 | NM_000981 | RPL19 | GGACCGTCACAGGCTTGCGGATGATCAGCC | 34 |
| | | | CATCTTTGATGAGCTTCCGG | |
| 35 | NM_012101 | TRIM29 | GTAGCAGATGCAGGTCTGGTCGGTCTGGCA | 35 |
| | | | GAAGAGCTCCATCGTCTTGC | |
| 36 | NM_005978 | S100A2 | GAAGGTAGTGACCAGCACAGCCAGCGCCTG | 36 |
| | | | CTCCAGAGAACTGCACATCA | |
| 37 | NM_198477 | CXCL17 | GTTTGAGAAATTGCTGGCAGGCTCTGGAAT | 37 |
| | | | GCTTGTTTGGCTTTCTGTGG | |
| 38 | NM_001114980 | DeltaNP63 | GAGACCCTTACAATATGAATCTACTTAAGA | 38 |
| | | | AGATAACAGAACTCAAGTCC | |
| 39 | NM_002274 | KRT13 | GTGAGAGCAGGATTGAGAGCAGGTGCAGAT | 39 |
| | | | AGAAGCTTGCTTGGCCTGGG | |
| 40 | NM_002456 | MUC1 | ACTGCTGGGTTTGTGTAAGAGAGGCTGCTG | 40 |
| | | | CCACCATTACCTGCAGAAAC | |
| 41 | NM_000299 | PKP1 | CAAAGCCAACGTGGAGTTGTCCTGGTCCTG | 41 |
| | | | GAAGCATTCGTACGCCAAGG | |
| 42 | NM_119937 | ANT | GCAAATACTATTTATACCGACGAAACTAAA | 42 |
| | | | CCGAATTGTAGTTCAAACCG | |
| 43 | NM_001161616 | RGL3 | CAGGAGAAGAGTCGCAAGCTTGCCTGTGGG | 43 |
| | | | ACTTGTGTCTTGTGGAGAGG | |
| 44 | NM_001941 | DSC3 | CCAGTTCAGGCTCATCCTGCAAATGCCTTC | 44 |
| | | | AGACTCATCATGCAGTCAGC | |
| 45 | NM_022121 | PERP | GATGTAAGTGACAGCAGGGTTGGCATGAAG | 45 |
| | | | GGTGAAGGTCTGGGTGTACT | |
| 46 | NM_005185 | CALML3 | GACACCAGCACACGGACAAACTCCTCGTAG | 46 |
| | | | TTCACCTGTCCGTCTCCGTC | |
| 47 | NM_002295 | RPSA | CCACCACATCAAACCCACTGAGTGAGCTCC | 47 |
| | | | CTTGTTGTTGCATGGGATGG | |

The NSCLC squamous/nonsquamous classifier described in Example 2 was used to classify each sample into squamous or nonsquamous NSCLC types. The NSCLC squamous/nonsquamous classifier provided results as shown in Table 9.

TABLE 9
Illustrative NSCLC Classifier Results.

| | NSCLC Squamous/Nonsquamous Classifier Result | |
|---|---|---|
| | Consistent with Squamous | Consistent with Non- |
| Tumor Subtype | NSCLC | Squamous NSCLC |
| Adenocarcinoma | | X |
| Squamous carcinoma | X | |
| Adenosquamous | X | |
| carcinoma | | |
| Large Cell Carcinoma | | X |

If the classifier was not able to definitively assign a NSCLC subtype then a result of “indeterminate” was provided. Classifier outputs were compared to the expert-panel consensus label to determine the error rate.

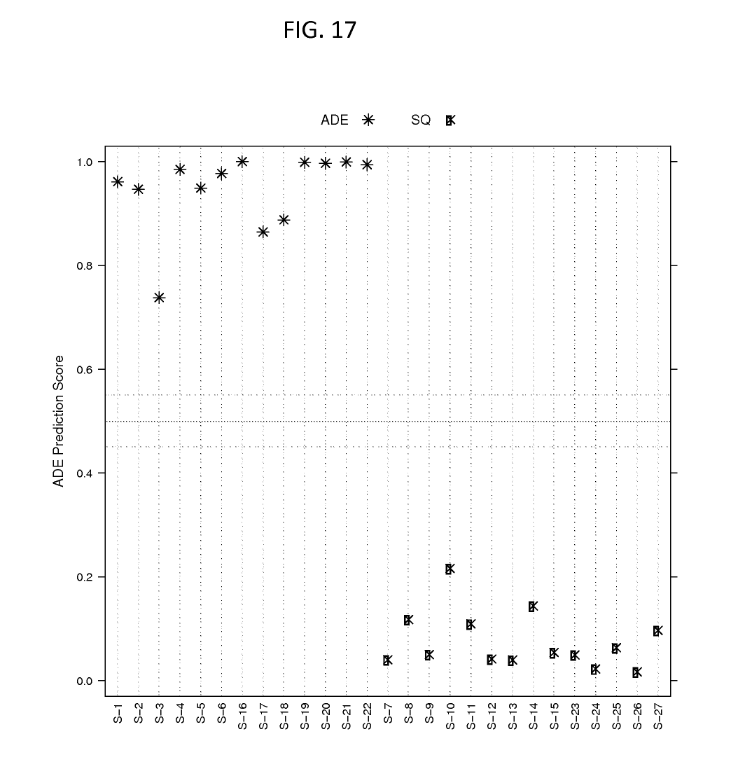

This representative classifier predicted the correct label, squamous or nonsquamous (e.g., adenocarcinoma), with 95% accuracy. The results for a subset of 27 samples (S-1 to S-27) are shown in FIG. 17.

It is thought that the genomes of some NSCLC adenocarcinomas may have anaplastic lymphoma kinase (ALK) gene rearrangements; such as fusions of the ALK gene with the echinoderm microtubule-associated protein-like 4 (EML4) gene. The discordant samples in this Example were further tested using a qNPA-based method that identifies a change in the relative expression of 5′ ALK mRNA and 3′ ALK mRNA and, thereby, identifies in a sample any expressed gene rearrangement wherein the 5′ portion of the ALK mRNA has been displace or replaced while the 3′ portion (and kinase-coding region) of the ALK mRNA remains intact (including ALK-EML4 fusions). Two of the five discordant samples tested positive for ALK gene rearrangements. This result supports further testing samples that are found to be indeterminant using a disclosed NSCLC squamous/nonsquamous classifier for the presence of an ALK gene rearrangement, such as an ALK/EML4 fusion event. A positive finding for ALK gene rearrangement indicates that such sample is a nonsquamous (or adenocarcinoma) NSCLC.

### Example 6

### Titration Analysis

Many types of biological samples contain a mixture of cell types. This is no less true in a biopsy or other biological sample taken for purposes of medical testing. The problem is that such sample heterogeneity can affect medical (e.g., diagnostic) test outcomes. For example, biopsies taken for tumor testing often contain adjacent normal tissue and, therefore, will vary in tumor content. Thus, a further aim of the present disclosure was to develop classifiers that not only distinguish between squamous and nonsquamous NSCLC accurately, but that are also robust to tumor impurity.

To determine the effect of tumor content on disclosed squamous/nonsquamous NSCLC classifiers, a series of mixed samples were created by titrating FFPE tissue lysates from well-adjudicated squamous NSCLC, adenocarcinoma NSCLC, and normal adjacent tissue (NAT) in varying proportions. The percentages of the various lysate types in the mixed samples are set forth in Table 10.

TABLE 10
Mixed Tumor and NAT Lysates

| | | Adenocarcinoma | |
|---|---|---|---|
| | | (Nonsquamous) | |
| | Squamous NSCLC | NSCLC | |
| Sample Name | (% Total) | (% Total) | NAT (% Total) |
| SQ-80-NAT-20 | 80 | | 20 |
| SQ-60-NAT-40 | 60 | | 40 |
| SQ-40-NAT-60 | 40 | | 60 |
| SQ-20-NAT-80 | 20 | | 80 |
| ADE-80-NAT-20 | | 80 | 20 |
| ADE-60-NAT-40 | | 60 | 40 |
| ADE-40-NAT-60 | | 40 | 60 |
| ADE-20-NAT-80 | | 20 | 80 |
| ADE-0-SQ-1000 | 100 | 0 | |
| ADE-10-SQ-90 | 90 | 10 | |
| ADE-20-SQ-80 | 80 | 20 | |
| ADE-40-SQ-60 | 60 | 40 | |
| ADE-50-SQ-50 | 50 | 50 | |
| ADE-60-SQ-40 | 40 | 60 | |
| ADE-80-SQ-20 | 20 | 80 | |
| ADE-100-SQ-0 | 0 | 100 | |

Gene expression information for the mixed samples was obtained and each sample was classified as squamous or nonsquamous (e.g., adenocarcinoma) NSCLC as set in Example 5.

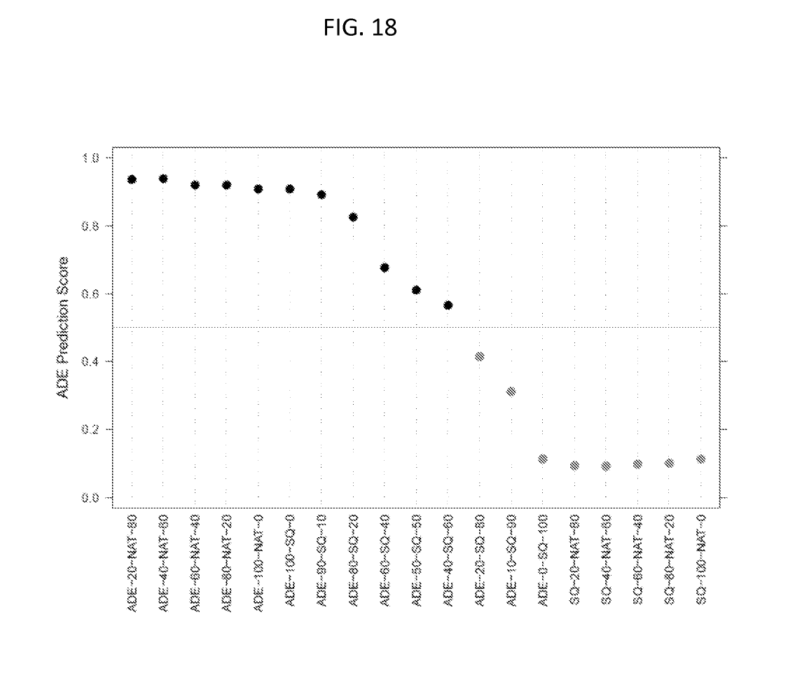

As shown in FIG. 18 (leftmost five data points), when adenocarcinoma lysate was combined with NAT lysate in the indicated proportions, all such mixtures were predicted to be adenocarcinoma (nonsquamous) with high probability. Similarly, all squamous and NAT lysate mixtures were predicted to be squamous with high probability (rightmost five data points in FIG. 18). Further, not only were all mixed tumor and NAT samples accurately classified as adenocarcinoma or squamous, the estimated probabilities of such classification did not vary significantly by dilution ratio. These results indicates that, when mixed with normal tissue, the NSCLC squamous/nonsquamous classifications are consistent and accurate despite varying amounts of squamous or nonsquamous tumor in a sample.

When adenocarcinoma and squamous lysates were titrated together, the adenocarcinoma prediction score decreased roughly linearly with the adenocarcinoma concentration. Thus, samples that were predominantly adenocarcinoma also had a high probably of being classified correctly, and samples that were predominantly squamous were very unlikely to be misclassified as adenocarcinoma. Samples that had more equal mixtures of adenocarcinoma and squamous lysates had modest prediction probability scores, which means that such samples would not be classified accurately as either cancer subtype. With regard to the mixed tumor experiments described in this paragraph, it is important to note that, clinically, it is unusual to find a significant proportion (e.g., 50:50, 60:40, 40:60) of both squamous and nonsquamous subtypes in the same NSCLC biopsy.

In summary, these Examples describe, among other things, representative and robust gene sets and NSCLC squamous/nonsquamous classifiers that provide reliable results in multiple independent experiments, using several distinct analytical methods, with samples from various sources to mimic the variability of a typical community hospital setting, and regardless of inherent sample related variation. These rigorous requirements for classifier discovery, training and validation eliminated genes that may seem correlated to a desired class in one given scenario by random chance, and focused on genes that convey genuine clinically relevant information.

While this disclosure has been described with an emphasis upon particular embodiments, it will be obvious to those of ordinary skill in the art that variations of the particular embodiments may be used and it is intended that the disclosure may be practiced otherwise than as specifically described herein. Accordingly, this disclosure includes all modifications encompassed within the spirit and scope of the disclosure as defined by the following claims:

We claim:

1. A method of characterizing a lung sample obtained from a subject, comprising:
obtaining from the sample raw expression values for each of at least two biomarkers in Table 2, 3, or 4 and at least one normalization biomarker(s);
normalizing the raw expression values for each of the at least two biomarkers in Table 2, 3, or 4 to the raw expression values for the at least one normalization biomarker(s) to produce normalized expression values for each of the at least two biomarkers in Table 2, 3, or 4;
combining the normalized expression values for each of the at least two biomarkers in Table 2, 3, or 4 to generate an output value;
comparing the output value to a cut-off value, wherein the cut-off value was determined by regression analysis of normalized expression values for the at least two biomarkers in Table 2, 3, or 4 in a plurality of NSCLC samples known in advance to be squamous cell NSCLC or nonsquamous cell NSCLC; and
characterizing the sample as squamous cell NSCLC if the output value is on the same side of the cut-off value as the plurality of known squamous cell NSCLC samples or characterizing the sample as nonsquamous cell NSCLC if the output value is on the same side of the cut-off value as the plurality of known nonsquamous cell NSCLC samples.

2. The method of claim 1, wherein the combining step comprises (a) weighting the expression level of the at least two biomarkers in Table 2, 3, or 4 with a constant predetermined for each of the at least two biomarkers in Table 2, 3, or 4, and (b) summing the weighted expression levels of the at least two biomarkers in Table 2, 3, or 4 to generate the output value.

3. The method of claim 1 or 2, wherein the at least one normalization biomarker(s) comprises a plurality of normalization biomarkers none of whose expression is statistically significantly different among a plurality of lung samples.

4. The method of claim 3, wherein the plurality of lung samples comprises squamous cell NSCLC, nonsquamous NSCLC, and large cell lung cancer, and, optionally, one or more of lung metastases of colon cancer, small cell lung cancer, or small cell lung cancer.

5. The method of any of claims 1 to 4, wherein the normalizing step comprises calculating a population central tendency from the raw expression values of the at least one normalization biomarker(s), and normalizing the raw expression values for each of the at least two biomarkers in Table 2, 3, or 4 to the population central tendency to produce normalized expression values for each of the at least two biomarkers in Table 2, 3, or 4.

6. The method of any of claims 1 to 5, wherein the at least one normalization biomarker(s) comprises 1, 2, 3, 4, 5, 6, 7, 8, 9 or 10 of the biomarkers in Table 7.

7. The method of any of claims 1 to 6, wherein the characterizing step comprises characterizing the sample as nonsquamous cell NSCLC if the output value is below the cut-off value or characterizing the sample as squamous cell NSCLC if the output value is above the cut-off value.

8. The method of any of claims 1 to 7, further comprising:
obtaining from the sample raw expression values for at least one colon metastasis biomarker in Table 5;
normalizing the raw expression values for each of the at least one colon metastasis biomarker(s) in Table 5 to the raw expression values for the at least one normalization biomarker(s) to produce normalized expression values for each of the at least one colon metastasis biomarker(s) in Table 5; and
identifying the sample as not NSCLC based on the normalized expression values for each of the at least one colon metastasis biomarker(s) in Table 5 and, optionally, removing the sample from further NSCLC subtyping.

9. The method of any of claims 1 to 8, further comprising:
obtaining from the sample raw expression values for at least one pulmonary carcinoid/small cell lung cancer biomarker in Table 6;
normalizing the raw expression values for each of the at least one pulmonary carcinoid/small cell lung cancer biomarker(s) in Table 6 to the raw expression values for the at least one normalization biomarker(s) to produce normalized expression values for each of the at least one pulmonary carcinoid/small cell lung cancer biomarker(s) in Table 6; and
identifying the sample as not NSCLC based on the normalized expression values for each of the at least one pulmonary carcinoid/small cell lung cancer biomarker(s) in Table 6 and, optionally, removing the sample from further NSCLC subtyping.

10. The method of claim 9, wherein:
the at least two biomarkers in Table 2, 3, or 4 comprise KRT5, CAPN8, DSG3, IRF6, KCNK5, CSTA, CLCA2, TJP3, TP63, KRT7, MIR205HG, CLDN3, CGN, NKX2-1, SERPINB5, SLC2A1, KRT6B, KRT6A, TRIM29, S100A2, DeltaNP63, KRT13, MUC1, PKP1, RGL3, DSC3, PERP, and CALML3,
wherein the at least one normalization biomarker(s) comprises RPS29, EEF2, DDX17, RPL19, RPSA, and HMGXB3;
wherein the at least one colon metastasis biomarker(s) in Table 5 comprises SFTPB, CLRN3, CDH17, LGALS4, and CXCL17, and
wherein the at least one pulmonary carcinoid/small cell lung cancer biomarker in Table 6 comprises CHGA, TSPYL2, APLP1, CAMK2B, TAGLN3, and NCAM1.

11. A method of characterizing a lung sample obtained from a subject, comprising:
obtaining from the sample raw expression values for at least one colon metastasis biomarker in Table 5;
normalizing the raw expression values for each of the at least one colon metastasis biomarker(s) in Table 5 to the raw expression values for the at least one normalization biomarker(s) to produce normalized expression values for each of the at least one colon metastasis biomarker(s) in Table 5; and
identifying the sample as not NSCLC based on the normalized expression values for each of the at least one colon metastasis biomarker(s) in Table 5.

12. A method of characterizing a lung sample obtained from a subject, comprising:
obtaining from the sample raw expression values for at least one pulmonary carcinoid/small cell lung cancer biomarker in Table 6;
normalizing the raw expression values for each of the at least one pulmonary carcinoid/small cell lung cancer biomarker(s) in Table 6 to the raw expression values for the at least one normalization biomarker(s) to produce normalized expression values for each of the at least one pulmonary carcinoid/small cell lung cancer biomarker(s) in Table 6; and
identifying the sample as not NSCLC based on the normalized expression values for each of the at least one pulmonary carcinoid/small cell lung cancer biomarker(s) in Table 6.

13. The method of any of claim 8, 9 or 11, wherein the at least one colon metastasis biomarker in Table 5:
comprises two or more of CDH17, LGALS4, CXCL17, SFTPA2, SCGB3A2, NAPSA, SFTPD, AQP4, SFTA3, SFTPC, CP, MUC13, HEPH, ZNF512B, and USH1C;
consists of CDH17, LGALS4, CXCL17, SFTPA2, SCGB3A2, NAPSA, SFTPD, AQP4, SFTA3, SFTPC, CP, MUC13, HEPH, ZNF512B, and USH1C;
comprises two or more of SFTPB, CLRN3, CDH17, LGALS4, and CXCL17; or
consists of SFTPB, CLRN3, CDH17, LGALS4, and CXCL17.

14. The method of claim 9 or 12, wherein the at least one pulmonary carcinoid/small cell biomarker in Table 6:
comprises two or more of CHGA, TSPYL2, APLP1, CAMK2B, TAGLN3, and NCAM1; or consists of CHGA, TSPYL2, APLP1, CAMK2B, TAGLN3, and NCAM1.

15. A method of determining gene expression in a lung sample, comprising:
obtaining a lung sample from a subject;
determining in the sample expression levels of a plurality of genes comprising at least two of the biomarkers in Table 2, 3, or 4; and
producing a report comprising at least one of the gene expression levels in the sample, or a characterization of the sample as squamous NSCLC or nonsquamous NSCLC or neither.

16. The method of claim 15, further comprising determining in the sample the expression levels of at least one normalization biomarker.

17. The method of claim any of claims 1 to 16, wherein the lung sample comprises a NSCLC sample.

18. The method of any of claims 1 to 17, wherein obtaining raw expression values is determined in a solution-based (ex situ) assay.

19. The method of claim 18, wherein the solution-based assay comprises PCR or a nuclease protection assay.

20. The method of any of claims 1 to 17, wherein obtaining raw expression values is determined in an in situ assay.

21. The method of claim 20, wherein the in situ assay comprises immunohistochemistry or in situ hybridization.

22. The method of any of claims 1 to 9 or 15 to 21, wherein the at least two biomarkers in Table 2, 3, or 4:
comprise KRT5, CAPN8, DSG3, IRF6, KCNK5, CSTA, CLCA2, TJP3, TP63, KRT7, MIR205HG, CLDN3, CGN, NKX2-1, SERPINB5, SLC2A1, KRT6B, KRT6A, TRIM29, S100A2, DeltaNP63, KRT13, MUC1, PKP1, RGL3, DSC3, PERP, and CALML3;
comprise DST, KRT17, NTRK2, SERPINB13, SLC6A8, SPRR1A, SPRR1B, SPRR3, or combinations thereof;
consist of KRT5, CAPN8, DSG3, IRF6, KCNK5, CSTA, CLCA2, TJP3, TP63, KRT7, MIR205HG, CLDN3, CGN, NKX2-1, SERPINB5, SLC2A1, KRT6B, KRT6A, TRIM29, S100A2, DeltaNP63, KRT13, MUC1, PKP1, RGL3, DSC3, PERP, and CALML3; or
consist of KRT5, CAPN8, DSG3, IRF6, KCNK5, CSTA, CLCA2, TJP3, TP63, KRT7, MIR205HG, CLDN3, CGN, NKX2-1, SERPINB5, SLC2A1, KRT6B, KRT6A, TRIM29, S100A2, DeltaNP63, KRT13, MUC1, PKP1, RGL3, DSC3, PERP, CALML3; KRT5, CAPN8, DSG3, IRF6, KCNK5, CSTA, CLCA2, TJP3, TP63, KRT7, MIR205HG, CLDN3, CGN, NKX2-1, SERPINB5, SLC2A1, KRT6B, and KRT6A.

23. The method of any of claim 1-14 or 17-22, further comprising comparing the output value to a reference value that distinguishes known squamous NSCLC samples from known nonsquamous NSCLC samples.

24. The method of claim 23, further comprising characterizing the sample as squamous NSCLC if the output value falls on the same side of the reference value as do the known squamous NSCLC samples.

25. The method of any of claims 1 to 9 or 17 to 21, wherein the at least two biomarkers in Table 2, 3, or 4 are at least 50%, at least 75%, at least 80%, at least 90%, at least 95% or at least 98% of a plurality of genes for which raw expression values are obtained.

26. The method of any of claim 15 or 16, wherein the at least two biomarkers in Table 2 or 3 are at least 50%, at least 75%, at least 80%, at least 90%, at least 95% or at least 98% of the plurality of genes for which expression levels are determined.

27. A method of subtyping NSCLC in a lung sample, comprising:
determining, in a lung sample obtained from a subject, an expression level of at least two biomarkers selected from: KRT5, CAPN8, DSG3, IRF6, KCNK5, CSTA, CLCA2, TJP3, TP63, KRT7, MIR205HG, CLDN3, CGN, NKX2-1, SERPINB5, SLC2A1, KRT6B, KRT6A, TRIM29, S100A2, DeltaNP63, KRT13, MUC1, PKP1, RGL3, DSC3, PERP, and CALML3;
calculating an output from an algorithm that uses the expression levels of the at least two biomarkers as an input; and
determining from the algorithm output that the sample is squamous NSCLC, nonsquamous NSCLC or not NSCLC by comparing the output to a reference standard obtained from samples of known squamous and nonsquamous NSCLC subtypes.

28. The method of claim 27, further comprising normalizing the expression levels of the at least two biomarkers to the expression level of at least one normalization biomarker selected from the group consisting of:
(a) at least one of EEF2, DDX17, HMGXB3, RPL19, RPS29 and/or RPSA;
(b) EEF2, DDX17, HMGXB3, RPL19, RPS29 and RPSA;
(c) all 11 biomarkers in Table 7; or
(d) at least one gene expressed in the lung sample that is not the at least two biomarkers, and the expression of which does not significantly differ in a representative plurality of lung samples.

29. The method of claim 28, wherein normalizing comprises log transforming raw expression values of the at least two biomarkers selected from: KRT5, CAPN8, DSG3, IRF6, KCNK5, CSTA, CLCA2, TJP3, TP63, KRT7, MIR205HG, CLDN3, CGN, NKX2-1, SERPINB5, SLC2A1, KRT6B, KRT6A, TRIM29, S100A2, DeltaNP63, KRT13, MUC1, PKP1, RGL3, DSC3, PERP, and CALML3, and the raw expression value(s) of the at least one normalization biomarker and dividing each of the at least two biomarkers log transformed raw expression values by the log transformed raw expression value(s) of the at least one normalization biomarker.

30. The method of any of claims 27 to 29, wherein the algorithm is
Algorithm Output=β0+β1X1+β2X2+ . . . βnXn
wherein Xn are the log transformed expression values for the at least two (up to n) biomarkers selected from: KRT5, CAPN8, DSG3, IRF6, KCNK5, CSTA, CLCA2, TJP3, TP63, KRT7, MIR205HG, CLDN3, CGN, NKX2-1, SERPINB5, SLC2A1, KRT6B, KRT6A, TRIM29, S100A2, DeltaNP63, KRT13, MUC1, PKP1, RGL3, DSC3, PERP, and CALML3, wherein 30 is greater than −200 and less than 200, wherein all β for n>0 are greater than −1,000 and less than 1,000.

31. The method of claim 30, wherein where all 13 for n>0 are greater than −100 and less than 100.

32. The method of any of claims 27 to 31, wherein the steps of calculating the output from the algorithm, and determining from the algorithm output that the sample is squamous NSCLC, nonsquamous NSCLC or neither by comparing the output to a reference standard are performed by a suitably programmed computer.

33. The method of any of claims 1 to 32, wherein determining an expression level comprises determining RNA expression.

34. The method of claim 33, wherein determining the RNA expression level or expression value comprises contacting the sample with a plurality of nucleic acid probes or paired amplification primers, wherein each probe or paired primers is/are specific and complementary to one of the least two biomarkers selected from: KRT5, CAPN8, DSG3, IRF6, KCNK5, CSTA, CLCA2, TJP3, TP63, KRT7, MIR205HG, CLDN3, CGN, NKX2-1, SERPINB5, SLC2A1, KRT6B, KRT6A, TRIM29, S100A2, DeltaNP63, KRT13, MUC1, PKP1, RGL3, DSC3, PERP, and CALML3, under conditions that permit the plurality of nucleic acid probes or paired primers to hybridize to its/their complementary at least two biomarkers.

35. The method of claim 34, further comprising, after contacting the sample with the plurality of nucleic acid probes, contacting the sample with a nuclease that digests single-stranded nucleic acid molecules.

36. The method of any of claims 1 to 32, wherein determining an expression level or expression value comprises determining protein expression.

37 The method of any one of claims 1 to 36, wherein the subject is a human.

38. The method of any of claims 1 to 37, wherein the lung sample is fixed.

39. The method of any of claims 1 to 9 or 13 to 38, wherein the at least two biomarkers in Table 2, 3, or 4 are contemporaneously determined in a plurality of samples obtained from different subjects.

40. The method of any of claims 1 to 39, wherein a prior-used method was unable to reliably determine if the lung sample was squamous NSCLC or nonsquamous NSCLC.

41. The method of claim 40, wherein the prior-used method is histopathology or immunohistochemistry.

42. The method of any one of claims 27-41, further comprising providing to a user a report comprising the algorithm output and/or the determination that the sample is squamous NSCLC, nonsquamous NSCLC, or is indeterminant.

43. The method of any one of claims 1 to 42, wherein if the lung sample is determined to be squamous NSCLC, the method further comprises selecting the subject for chemotherapy treatment.

44. The method of claim 43, further comprising treating the subject with chemotherapy.

45. The method of any one of claims 1 to 42, wherein if the lung sample is determined to be non-squamous NSCLC, the method further comprises selecting the subject for treatment with pemetrexed, bevacizumab, erlotinib, or crizotinib.

46. The method of claim 45, further comprising treating the subject with pemetrexed, bevacizumab, erlotinib, or crizotinib.

47. An array, comprising:
at least three addressable locations, each location comprising immobilized capture probes having the same specificity, and each location comprising capture probes having specificity different than capture probes at each other location,
wherein the capture probes at two of the at least three locations are capable of directly or indirectly specifically hybridizing a biomarker listed in Table 2, 3, or 4, and the capture probes at one of the at least three locations is capable of directly or indirectly specifically hybridizing a normalization biomarker listed in Table 7 and
wherein the specificity of each capture probe is identifiable by the addressable location the array.

48. The array of claim 47, wherein the at least three addressable locations each are a separately identifiable bead or a channel in a flow cell.

49. The array of claim 47 or 48, further comprising at least two discrete regions, each region comprising the at least three addressable locations.

50. The array of any of claims 47 to 49, wherein the array comprises immobilized capture probes capable of directly or indirectly specifically hybridizing with all 28 biomarkers listed in Table 3 and the first 6 normalization biomarkers in Table 7.

51. The array of any of claims 47 to 50, wherein the array further comprises capture probes capable of directly or indirectly specifically hybridizing to at least one colon metastasis biomarker listed in Table 5, and/or capture probes capable of directly or indirectly specifically hybridizing to at least one pulmonary carcinoid/small cell lung cancer biomarker listed in Table 6.

52. The array of any of claims 47 to 51, wherein the array comprises:
immobilized capture probes capable of directly or indirectly specifically hybridizing with the 28 biomarkers listed in Table 3;
immobilized capture probes capable of directly or indirectly specifically hybridizing with the first 6 normalization biomarkers in Table 7;
immobilized capture probes capable of directly or indirectly specifically hybridizing with at least five colon metastasis biomarkers comprising SFTPB, CLRN3, CDH17, LGALS4, and CXCL17, and
immobilized capture probes capable of directly or indirectly specifically hybridizing with pulmonary carcinoid/small cell lung cancer biomarkers comprising CHGA, TSPYL2, APLP1, CAMK2B, TAGLN3, and NCAM1.

53. The array of any of claims 47 to 52, wherein the array further comprises:
immobilized capture probes capable of directly or indirectly specifically hybridizing with a positive control; and
immobilized capture probes capable of directly or indirectly specifically hybridizing with a negative control.

54. The array of any of claims 47 to 50, wherein the capture probe(s) indirectly hybridize with the at least two biomarkers listed in Table 2, 3, or 4 and the at least one normalization biomarker in Table 7 through a nucleic acid programming linker, wherein the programming linker is a hetro-bifunctional linker which has a first portion complementary to the capture probe(s) and a second portion complementary to a nuclease protection probe (NPP), wherein the NPP is complementary to one of the at least two biomarkers listed in Table 2, 3, or 4 or the at least one normalization biomarker in Table 7.

55. The array of claim 52, wherein the capture probe(s) indirectly hybridize with the 28 biomarkers listed in Table 3, the first 6 normalization biomarkers in Table 7, the at least five colon metastasis biomarkers comprising SFTPB, CLRN3, CDH17, LGALS4, and CXCL17, and the pulmonary carcinoid/small cell lung cancer biomarkers comprising CHGA, TSPYL2, APLP1, CAMK2B, TAGLN3, and NCAM1, through a nucleic acid programming linker, wherein the programming linker is a hetro-bifunctional linker which has a first portion complementary to the capture probe(s) and a second portion complementary to a nuclease protection probe (NPP), wherein the NPP is complementary to one of the 28 biomarkers listed in Table 3, the first 6 normalization biomarkers in Table 7, the at least five colon metastasis biomarkers comprising SFTPB, CLRN3, CDH17, LGALS4, and CXCL17, or the pulmonary carcinoid/small cell lung cancer biomarkers comprising CHGA, TSPYL2, APLP1, CAMK2B, TAGLN3, and NCAM1.

56. The array of claim 53, wherein the capture probe(s) indirectly hybridize with the positive control and the negative control, through a nucleic acid programming linker, wherein the programming linker is a hetro-bifunctional linker which has a first portion complementary to the capture probe(s) and a second portion complementary to a nuclease protection probe (NPP), wherein the NPP is complementary to the positive control or the negative control.

57. The array of any of claims 47 to 56, wherein the at least two discrete regions are wells on a multi-well surface, or channels in a flow cell.

58. The array of any of claims 54 to 56, further comprising the nucleic acid programming linkers.

59. A kit, comprising:
the array of any one of claims 47 to 58, and
one or more of:
a container containing lysis buffer;
a container containing a nuclease specific for single-stranded nucleic acids;
a container containing a plurality of nucleic acid programming linkers;
a container containing a plurality of NPPs;
a container containing a plurality of the bifunctional detection linkers;
a container containing a detection probe that specifically binds the bifunctional detection linkers; and
a container containing a detection reagent.

60. The kit of claim 59, wherein the detection probe is triple biotinylated.

61. The kit of claim 59 or 60, wherein the detection reagent comprises avidin- or streptavidin-conjugated horseradish peroxidase (HRP).

62. The kit of any of claims 9 to 61, wherein the programming linkers are pre-hybridized to the capture probes.

63. The kit of any one of claims 59 to 62, wherein the kit further comprises control nucleic acids.


SEQUENCE LISTING (47 sequences)

SEQ ID NO: 1 - DNA, 50 bp, Homo Sapiens
ctgccaagtaattcgcaggcttcttcggccttatttaacaaggagtgctg

SEQ ID NO: 2 - DNA, 50 bp, Homo Sapiens
ggcagtgacttgcagcaggttcttagctcttgaagctcttccgggaggag

SEQ ID NO: 3 - DNA, 50 bp, Homo Sapiens
gaagaaagcttgcccggtcgccatcccatcatgccagagcgtgcagtgtc

SEQ ID NO: 4 - DNA, 50 bp, Homo Sapiens
caaatgctgtcacagatgacagtcacgttaccatgctgaattgtccagag

SEQ ID NO: 5 - DNA, 50 bp, Homo Sapiens
ataaatggaatccaggcagttctatgagacaacactgatatatctccttg

SEQ ID NO: 6 - DNA, 50 bp, Homo Sapiens
ccaagccacagcgaatggacagatcaaagaactgtccgggaccaaatggg

SEQ ID NO: 7 - DNA, 50 bp, Homo Sapiens
cttgatggtgatgcaacgctcctgctcgtccttccgggtatcagtgaagc

SEQ ID NO: 8 - DNA, 50 bp, Homo Sapiens
gaacctggtagacggcatagccgatgctaagcatgccttgtcctatccgc

SEQ ID NO: 9 - DNA, 50 bp, Homo Sapiens
caatggccgacaggctctccagctcctggtcctctggtctgcggtttgcg

SEQ ID NO: 10 - DNA, 50 bp, Homo Sapiens
gatgtcctcatcttgctggatgccttcctcaatgccctcttcttccactg

SEQ ID NO: 11 - DNA, 50 bp, Homo Sapiens
ggaacctcctcaggacttaccaggctgctgcacaggatctagcttctccc

SEQ ID NO: 12 - DNA, 50 bp, Homo Sapiens
gctcatccctctgaatctccgatgagtggaaagggaaaccccttggaaca

SEQ ID NO: 13 - DNA, 50 bp, Homo Sapiens
gacaagcaggttcgtgctaggtgagcctttccagaagagtactgcccagc

SEQ ID NO: 14 - DNA, 50 bp, Homo Sapiens
gatacgtggcctttgctcctcagtgtctgtgacacctcttccaaggtggg

SEQ ID NO: 15 - DNA, 50 bp, Homo Sapiens
catgactcagtagccagttgaaggaatcagaacactttgggtacatgctg

SEQ ID NO: 16 - DNA, 50 bp, Homo Sapiens
cccttcaaccaggttgcccagtgcttcaggctcaaacgaggtcagccctg

SEQ ID NO: 17 - DNA, 50 bp, Homo Sapiens
cagcctgtacaagcgagaatgtgggaggaggtggaagctcagtcccattc

SEQ ID NO: 18 - DNA, 50 bp, Homo Sapiens
gcaccagcaggcttagcttcccttctgacttctcaatcagtcgccgggtg

SEQ ID NO: 19 - DNA, 50 bp, Homo Sapiens
cactagtggctttgtgcctttgagcagttgggtctctgagccaaagtgtc

SEQ ID NO: 20 - DNA, 50 bp, Homo Sapiens
ccacagatgtgtcggagatctgggactgcagctctgtcaactccgtctca

SEQ ID NO: 21 - DNA, 50 bp, Homo Sapiens
tttccaatctgcccatcacccgtccctctgaagaagcacgcacactccag

SEQ ID NO: 22 - DNA, 50 bp, Homo Sapiens
ctgactcaccgacggcgcgcgctaacggctcggctccatacgctctcgcc

SEQ ID NO: 23 - DNA, 50 bp, Homo Sapiens
ccttcaccctcaggcttagctggtctttctggtcattgacatgctgccgc

SEQ ID NO: 24 - DNA, 50 bp, Homo Sapiens
agttctaaatgaacaggaggtggcagcagtaggcaacgcggattctcggc

SEQ ID NO: 25 - DNA, 50 bp, Homo Sapiens
ttacaagcgagtcctctttgctggcagagtgtgcccagagtgaagtttgg

SEQ ID NO: 26 - DNA, 50 bp, Homo Sapiens
ctattctgagggcctgtgcatttgaaccagagatctgtgcaggccctaga

SEQ ID NO: 27 - DNA, 50 bp, Homo Sapiens
cagcccattgcttaatacattggaaccctttccctaagttgagtttcaac

SEQ ID NO: 28 - DNA, 50 bp, Homo Sapiens
ggaagagcaagagagaaatgcatccctccttcagggagaatcaagagccc

SEQ ID NO: 29 - DNA, 50 bp, Homo Sapiens
catttgcagtgtcacctttagcacccacttgagcaagtgacagagaggtg

SEQ ID NO: 30 - DNA, 50 bp, Homo Sapiens
cggaaacactggcggcacatattgaggccatatttccggatcagaccgtg

SEQ ID NO: 31 - DNA, 50 bp, Homo Sapiens
caggacccacttcaaagaaggccacaaagccaaagatggccacgatgctc

SEQ ID NO: 32 - DNA, 50 bp, Homo Sapiens
gaatgcagactgcatcagaaggtacatcacttgccattcagggacactgc

SEQ ID NO: 33 - DNA, 50 bp, Homo Sapiens
gaaggtgagcttgcaggttgggaagggctgggctttaccaacagtgagat

SEQ ID NO: 34 - DNA, 50 bp, Homo Sapiens
ggaccgtcacaggcttgcggatgatcagcccatctttgatgagcttccgg

SEQ ID NO: 35 - DNA, 50 bp, Homo Sapiens
gtagcagatgcaggtctggtcggtctggcagaagagctccatcgtcttgc

SEQ ID NO: 36 - DNA, 50 bp, Homo Sapiens
gaaggtagtgaccagcacagccagcgcctgctccagagaactgcacatca

SEQ ID NO: 37 - DNA, 50 bp, Homo Sapiens
gtttgagaaattgctggcaggctctggaatgcttgtttggctttctgtgg

SEQ ID NO: 38 - DNA, 50 bp, Homo Sapiens
gagacccttacaatatgaatctacttaagaagataacagaactcaagtcc

SEQ ID NO: 39 - DNA, 50 bp, Homo Sapiens
gtgagagcaggattgagagcaggtgcagatagaagcttgcttggcctggg

SEQ ID NO: 40 - DNA, 50 bp, Homo Sapiens
actgctgggtttgtgtaagagaggctgctgccaccattacctgcagaaac

SEQ ID NO: 41 - DNA, 50 bp, Homo Sapiens
caaagccaacgtggagttgtcctggtcctggaagcattcgtacgccaagg

SEQ ID NO: 42 - DNA, 50 bp, Homo Sapiens
gcaaatactatttataccgacgaaactaaaccgaattgtagttcaaaccg

SEQ ID NO: 43 - DNA, 50 bp, Homo Sapiens
caggagaagagtcgcaagcttgcctgtgggacttgtgtcttgtggagagg

SEQ ID NO: 44 - DNA, 50 bp, Homo Sapiens
ccagttcaggctcatcctgcaaatgccttcagactcatcatgcagtcagc

SEQ ID NO: 45 - DNA, 50 bp, Homo Sapiens
gatgtaagtgacagcagggttggcatgaagggtgaaggtctgggtgtact

SEQ ID NO: 46 - DNA, 50 bp, Homo Sapiens
gacaccagcacacggacaaactcctcgtagttcacctgtccgtctccgtc

SEQ ID NO: 47 - DNA, 50 bp, Homo Sapiens
ccaccacatcaaacccactgagtgagctcccttgttgttgcatgggatgg